(19) United States
(12) Patent Application Publication
(10) Pub. No.: US 2025/0281930 A1
(43) Pub. Date: Sep. 11, 2025

(54) INERTIAL MICROFLUIDIC SEPARATION SYSTEM

(71) Applicant: CFD Research Corporation, Huntsville, AL (US)

(72) Inventors: Lap Man Lee, III, Madison, AL (US); Ketan Harendrakumar Bhatt, Huntsville, AL (US); Balabhaskar Prabhakarpandian, Madison, AL (US)

(21) Appl. No.: 19/216,427

(22) Filed: May 22, 2025

Related U.S. Application Data
(63) Continuation-in-part of application No. 17/313,830, filed on May 6, 2021, now Pat. No. 12,318,524.
(60) Provisional application No. 63/022,180, filed on May 8, 2020.

(51) Int. Cl. B01L 3/00; G01N 33/49

(52) U.S. Cl. CPC B01L 3/502753; G01N 33/491; B01L 2200/0652; B01L 2300/0864

(57) ABSTRACT

A microfluidic separation system may include an inlet configured to receive a carrier fluid including first particles. A carrier fluid sorter may be coupled to the inlet. The carrier fluid sorter may include a microfluidic network having a sorter constricted region with a first cross-sectional dimension and a sorter expansion region with a larger second cross-sectional dimension. At least one sorter side channel may be formed into a side of the sorter expansion region to receive a first fraction of the carrier fluid that may be first particle poor. At least one sorter outlet may be positioned downstream or medial from the sorter side channel to receive a second fraction of the carrier fluid that may be first particle concentrated. The microfluidic may be configured to direct the first fraction to the sorter side channel and the second fraction to the sorter outlet.

### CROSS-REFERENCE TO RELATED APPLICATIONS

This patent application is a continuation-in-part of U.S. patent application Ser. No. 17/313,830 filed on May 6, 2021, now U.S. Pat. No. 12,318,524 which claims the benefit of U.S. Provisional Application No. 63/022,180 filed May 8, 2020, each of which is incorporated herein by specific reference in its entirety.

### U.S. GOVERNMENT RIGHTS

This invention was made with government support under N00014-18-C-7004 awarded by the United States Navy, W81XWH-17-C-0177 awarded by the United States Army, HT942523C0008 awarded by the Defense Health Agency (DHA), and HT942523C0052 awarded by the DHA. The government has certain rights in the invention.

### BACKGROUND

Particle separation and concentration techniques—such as centrifugation, filtration, sedimentation, and chromatography—may be used for isolating and/or extracting particles. While effective for bulk separation, these techniques face challenges when handling small or similarly-sized particles leading to poor separation efficiency. They often require long processing times, bulky equipment, and multiple steps that may damage the particles or result in low purity due to co-isolation of contaminants. Additionally, limited resolution and lack of real-time control hinder precision and scalability, making them less suitable for high-throughput or clinical applications.

The separation of biological particles presents particular challenges due to their size range, fragility, and often complex compositions. Bacteria typically range from 0.5 to 5 micrometers, while viruses and bacteriophages may be much smaller (20-500 nanometers). Extracellular vesicles and exosomes may be even smaller, typically between 20 and 150 nanometers.

Thus, prior particle separation techniques may not be suitable for integration with microfluidic systems due to their size, cost, low precision, and reliance on high-force or batch processes that may damage particles. Accordingly, there is a need for a microfluidic separation technology that may separate and/or concentrate biological particles that may be employed in resource limited environments and that may reduce damage to biological particles during separation and/or concentration.

### SUMMARY

In some embodiments, a microfluidic separation system may include an inlet configured to receive a carrier fluid from a carrier fluid source, the carrier fluid including first particles. The system may include a carrier fluid sorter coupled to the inlet. The carrier fluid sorter may include a carrier fluid sorter microfluidic network. The carrier fluid sorter microfluidic network may have a sorter constricted region having a first cross-sectional dimension. The carrier fluid sorter microfluidic network may have a sorter expansion region having a second cross-sectional dimension that is larger than the first cross-sectional dimension. The carrier fluid sorter microfluidic network may include at least one sorter side channel formed into a side of the sorter expansion region. The at least one sorter side channel may be configured to receive a first fraction of the carrier fluid, the first fraction being first particle poor. The carrier fluid sorter microfluidic network may include at least one sorter outlet that is downstream or medial from the at least one sorter side channel. The at least one sorter outlet may be configured to receive a second fraction of the carrier fluid, the second fraction being first particle concentrated. The microfluidic network may be configured to direct the first fraction of the carrier fluid to the at least one sorter side channel and configured to direct the second fraction of the carrier fluid to the at least one sorter outlet.

In some embodiments, the carrier fluid may further include second particles that are smaller than the first particles, the first fraction being second particle concentrated and the second fraction being second particle poor. In some embodiments, the sorter constricted region may be defined by a width between about 2 micrometers and about 30 micrometers, a length between about 0.2 micrometers and about 2 centimeters, and a height between about 10 micrometers and about 500 micrometers. In some embodiments, a flow rate through the carrier fluid sorter microfluidic network may be between about 2 microliters per minute and about 1 milliliter per minute.

In some embodiments, the first particles may be virus particles. The carrier fluid may further include second particles that are smaller than the first particles, the first fraction being second particle concentrated and the second fraction being second particle poor. The second particles may be endotoxins or other virus particles. The sorter constricted region may have at least one of: a width between about 2 micrometers and about 10 micrometers, a length between about 0.2 millimeters and about 2 centimeters, or a height between about 10 micrometers and about 250 micrometers.

In some embodiments, the first particles may be a first group of virus particles and the second particles may be a second group of virus particles. The first group of virus particles being larger than the second group of virus particles. The sorter constricted region may have at least one of: a width between about 2 micrometers and about 10 micrometers, a length between about 0.2 millimeters and about 2 centimeters, or a height between about 10 micrometers and about 250 micrometers. The first group of virus particles may have a size between about 250 nanometers and about 530 nanometers and the second group of virus particles may have a size between about 40 nanometers and about 300 nanometers.

In some embodiments, the first particles may include bacteria. The carrier fluid may further include second particles that are smaller than the first particles, the first fraction being second particle concentrated and the second fraction being second particle poor. The second particles may be bacteriophages. A height of the at least one sorter side channel may be lesser than a height of the at least one sorter outlet to increase fluidic resistance provided by the at least one sorter side channel such that the first fraction of the carrier fluid may be directed to the at least one sorter side channel and the second fraction of the carrier fluid may be directed to the at least one sorter outlet. The sorter constricted region may have at least one of: a width between about 7 micrometers and about 20 micrometers, a length between about 0.2 millimeters and about 4 centimeters, or a height between about 40 micrometers and about 500 micrometers.

In some embodiments, the first particles may be liposomes, extracellular vesicles (EV), or exosomes. The carrier fluid may further include second particles that are smaller than the first particles, the first fraction being second particle concentrated and the second fraction being second particle poor. The second particles may be nucleic acids, proteins, or polypeptides. The sorter constricted region may have at least one of: a width of about 2 micrometers and about 10 micrometers, a length between about 0.2 millimeters and about 2 centimeters, or a height between about 10 micrometers and about 250 micrometers.

In some embodiments, the carrier fluid may be whole blood, the first particles may be red blood cells and white blood cells. The carrier fluid may include second particles that are smaller than the first particles, the first fraction being second particle concentrated and the second fraction being second particle poor. The second particles may be platelets. The sorter constricted region may have at least one of: a width between about 10 micrometers and about 30 micrometers, a length between about 0.5 millimeters and about 30 millimeters, or a height between about 25 micrometers and about 500 micrometers.

In some embodiments, the carrier fluid may be platelet-rich plasma and the first particles may be platelets. The sorter constricted region may have at least one of: a width between about 10 micrometers and about 30 micrometers, a length between about 0.5 millimeters and about 30 millimeters, or a height between about 25 micrometers and about 500 micrometers.

In some embodiments, a method of separating particles may include receiving carrier fluid at an inlet of a carrier fluid sorter, the carrier fluid including first particles. The method may include fractionating the carrier fluid into a first fraction and a second fraction, the first fraction being first particle poor and the second fraction being first particle concentrated. The method may include directing the first fraction to at least one sorter side channel of the carrier fluid sorter. The method may include directing the second fraction to at least one sorter outlet that is downstream or medial from the at least one sorter side channel.

In some embodiments, the carrier fluid may include second particles that are smaller than the first particles, the first fraction may be second particle concentrated and the second fraction may be second particle poor. In some embodiments, the first particles may be virus particles and the second particles may be endotoxins or different virus particles. The first particles may be a first group of virus particles and the second particles may be a second group of virus particles, the first group of particles being larger than the second group of particles. The first particles may be bacteria and the second particles may be bacteriophages. The first particles may be liposomes, extracellular vesicles (EV), or exosomes and the second particles may be nucleic acids, proteins, or polypeptides. The first particles may be red blood cells and white bloods cells and the second particles may be platelets. In some embodiments, the first particles may be platelets.

In some embodiments, the fractionation of the carrier fluid may be attributable, at least in part, to a sorter constricted region downstream of the inlet and upstream of the at least one sorter side channels and the at least one sorter outlet, the sorter constricted region being defined by a width between about 2 micrometers and about 30 micrometers, a length between about 0.2 micrometers and about 2 centimeters, and a height between about 10 micrometers and about 500 micrometers.

In some embodiments, the methods described throughout this disclosure may be performed by the microfluidic separation systems described throughout this disclosure.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 illustrates an example of an embodiment of an inertial microfluidic separation system configured as a portable apheresis system.

FIG. 2A is a top view or longitudinally oriented cross-sectional view of a platelet separator channel that can be used for separating or concentrating platelets from whole blood.

FIG. 2B includes two cross-sectional views (laterally oriented) of the platelet separator channel of FIG. 2A at A-A′ and B-B′.

FIG. 3A shows an embodiment of a microfluidic network having the inlet (INL), platelet margination channel with the outlet expansion channel (e.g., expanded region) with a plurality of side channels (e.g., side channels O1, O2, O3, O4, O5, O6, O7, O8, and O9).

FIG. 3B shows the margination of Calcein AM stained PLTs in the expansion channel at flow rate ranging from 20 μL/min to 80 μL/min.

FIG. 3C shows an intensity profiles at 80 μL/min (Dotted line-Intensity profile for PLTs acquired using fluorescence imaging; Solid Line-Intensity profile for RBCs acquired using bright-field imaging).

FIG. 4 shows the schematic of the carrier fluid sorter configured for PLT extraction with HAR straight microfluidic channel.

FIG. 5A shows the inertial focusing of red blood cells (RBCs) and white blood cells (WBCs) in the expansion channel close to the outlet.

FIG. 5B shows the cell count (Square: WBC; Triangle: PLT, and Circle: RBC) counting under cell counting chamber.

FIG. 5C shows the PLT ratio and enrichment plots (INL is inlet, PRP is platelet rich portion, RTN is return to patient).

FIG. 5D shows the recovery plot (PRP is platelet rich portion, RTN is return to patient).

FIG. 6A shows a biological particle concentrator configured as a PLT concentrator that includes the inlet (INL) that feeds a focusing channel (e.g., a sorter constricted region) into a separator region (e.g., outlet expansion channel) that has the arranged outlet lines that correspond with outlets O1 through O9.

FIG. 6B shows the focusing and collection of Calcein AM stained human PLTs in the expansion channel at different flow rates 40-160 μL/min.

FIG. 6C shows the collection enriched/depleted human PLTs at different outlets at average flow rate of 120 μL/min.

FIG. 7A shows Scheme I, where the PRP extracted from all carrier fluid sorter units is collected and processed with one common PLT concentrator unit.

FIG. 7B shows Scheme II, where each PLT extraction unit consists of a pair of a whole blood sorter and a PLT concentrator, where the PPP is re-injected to the next unit to improve PLT recovery and extraction efficiency.

FIG. 8 shows an example of serial carrier fluid sorting, PLT extraction and concentration in batch processing mode: (a) 1^{st} PLT extraction from WB Sort1 (sorter); (b) 2^{nd} PLT extraction from WB Sort2 (sorter); and (c) PLT concentration from PLT Conc1 (concentrator) from both WB Sort1 and WB Sort2.

FIG. 9 shows a microfluidic design of Scheme 1 in continuous operation mode.

FIG. 10 plots the total PLT extraction efficiency with respect to the number of carrier fluid sorters connected in series.

FIG. 11A shows a photomask layout of a microfluidic separation system configured as a platelet apheresis microfluidic plate.

FIG. 11B shows that each branch containing four serially connected carrier fluid sorters configured as HAR WB sorters connected to a common inlet at the center.

FIG. 11C shows that PLTs are collected by the “PRP” outlet on the side.

FIG. 11D shows that the “PRP/RTN” outlet volume ratio of each HAR sorter is designed to be 1:11 (the volume each “PRP” side outlet, Arm1 is 1:22 of the “RTN” outlet, Arm 2).

FIG. 12A includes flow cytometry data for PLT activation biomarkers.

FIG. 12B includes ELISA data for PLT activation biomarkers.

FIG. 13 includes a schematic diagram of a computing device that can operate as a controller of the systems described herein.

FIG. 14A shows the mask for the tall central channel region containing multiple sections of the constricting channel followed by expansion regions.

FIG. 14B is the mask layer for the shallow side extraction channels that add fluidic resistance to the platelet extraction arms.

FIG. 14C is the overlay of the two layers of FIG. 14A and FIG. 14B.

FIG. 15A includes a schematic that shows each branch contains five serial expansion sections from the inlet INL to the return RTN or platelet rich plasma PRP.

FIG. 15B includes a schematic that shows the side extraction channels were designed with a shallower height compared with the main microfluidic channel along the centerline.

FIG. 16A shows RBC flow trajectories in real-time were imaged under an optical microscope at different locations of the device at one branch.

FIG. 16B shows the sorting performance data.

FIG. 17 shows a PDMS manifold that connects all common outlets from the fluidics layer of the microfluidic platelet extraction plates.

FIG. 18A shows the platelet apheresis cartridges can be operated using dual syringe pumps.

FIG. 18B shows valve actuation steps for the different processing steps: priming, and running, which can be controlled by the computing device of FIG. 13.

FIG. 19 is a top view or longitudinally oriented cross-sectional view of a microfluidic network that may be used for biological particle (e.g., phage particle) separation or concentration.

FIG. 20 is a schematic showing the variation in lift relative to the side wall of the microfluidic network for different particle sizes as the particles progress through the microfluidic network.

FIG. 21 illustrates predicted flow trajectories of different size pairs of phage particles.

FIG. 22 illustrates the predicted separation at the outlet for different phage particle size pairs.

FIG. 23 illustrates expected flow rates of increasing channel height in a microfluidic separation system configured for phage separation.

FIG. 24A illustrates an example microfluidic separation system with multiple carrier fluid sorters connected in series.

FIG. 24B illustrates an equivalent electrical circuit for the microfluidic separation system of FIG. 24.

FIG. 24C illustrates expected total pressure for different carrier fluid sorter microfluidic network configurations.

FIG. 25A shows the separation of a first group of particles having a size of 530 nm and a second group of particles having a size of 50 nm in an expansion region of a microfluidic separation system having a focusing channel (e.g., a sorter constricted region) of 7 micrometer width, 120 micrometer length, and 50 micrometer height at a flowrate of 60 μL/min.

FIG. 25B illustrates fluorescent images of samples of carrier fluid taken at an inlet and at different outlets in the microfluidic separation system of FIG. 25A.

FIG. 26A shows the separation of particles having a size of 300 nm and a size of 50 nm in an expansion region of a microfluidic separation system having a focusing channel of 5 micrometer width, 120 micrometer length, 50 micrometer height and a volume split ratio of 29:1 (O2:O1) at a flowrate of 40 L/min.

FIG. 26B illustrates fluorescent images of samples of carrier fluid taken at an inlet and at different outlets in the microfluidic separation system of FIG. 26A.

FIG. 27A shows the separation of a first group of particles having a size of 530 nm and a second group of particles having a size of 300 nm in an expansion region of a microfluidic separation system having a focusing channel of 7 micrometer width, 120 micrometer length, 50 micrometer height, and a volume split ratio of 24:1 (O2:O1) at a flowrate of 40 μL/min.

FIG. 27B illustrates fluorescent images of samples of carrier fluid taken at an inlet and at different outlets in the microfluidic separation system of FIG. 27A.

FIG. 28A shows the separation of a first group of particles having a size of 250 nm and a second group of particles having a size of 40 nm in an expansion region of a microfluidic separation system having a focusing channel of 5 micrometer width, 120 micrometer length, 50 micrometer height, and a splitting ratio of 24:1 (O2:O1) at a flowrate of 40 μL/min.

FIG. 28B illustrates fluorescent images of samples of carrier fluid taken at an inlet and at different outlets in the microfluidic separation system of FIG. 28A.

FIG. 29A shows the separation of a first group of particles having a size of 300 nm and a second group of particles having a size of 200 nm in an expansion region of a microfluidic separation system having a focusing channel of 5 micrometer width, 120 micrometer length, 50 micrometer height, and a volume split ratio of 24:1 (O2:O1) at a flowrate of 40 μL/min.

FIG. 29B illustrates fluorescent images of samples of carrier fluid taken at an inlet and at different outlets in the microfluidic separation system of FIG. 29A.

FIG. 30A shows the separation of a LUZ19 Phage particles and FITC conjugated LPS endotoxins in an expansion region of a microfluidic separation system having a focusing channel of 10 micrometer width, 1 centimeter length and volume split ratio 3.3:1 (O2:O1) at a flow rate of 40 μL/min.

FIG. 30B shows the separation of a LUZ19 Phage particles and FITC conjugated LPS endotoxins in the microfluidic separation system of FIG. 30A in a fourth cycle of the carrier fluid through the microfluidic separation system.

FIG. 31 shows intensity profiles along the A-A′ line illustrated in FIG. 30A for the LUZ19 phage particles and the FITC conjugated LPS endotoxins in the expansion region at varying flow rates.

FIG. 32A illustrates a configuration of a microfluidic separation system in which fluidic resistance in the sorter side outlets (O1, O3) is manipulated by resistors to alter separation efficiency.

FIG. 32B shows the separation of a LUZ19 Phage particles and FITC conjugated LPS endotoxins in the expansion region of the microfluid separation system of FIG. 32A having a focusing channel of 10 micrometer width, 2 centimeter length and volume split ratio 3.3:1 (O2:O1) at a flow rate of 20 μL/min in which 100 micrometer ID PEEK tubes with 4.5 cm length were inserted into the sorter side outlets (O1, O3).

FIG. 32C shows the separation of a LUZ19 Phage particles and FITC conjugated LPS endotoxins in the expansion region of the microfluidic separation system of FIG. 32A having a focusing channel of 10 micrometer width, 2 centimeter length and volume split ratio 3.3:1 (O2:O1) at a flow rate of 40 μL/min in which 100 micrometer ID PEEK tubes with 16 cm length were inserted into the sorter side outlets (O1, O3).

FIG. 33A shows the separation of PY02 phage particles from YO nanoparticles having a size of 200 nm in an expansion region of a microfluidic separation system at a flow rate of 5 μL/min.

FIG. 33B illustrates the intensity profile along the A-A′ line illustrated in FIG. 33A for the PY02 phage particles and the YO 200 nm nanoparticles.

FIG. 33C illustrates fluorescent images of samples of carrier fluid taken at an inlet and at different outlets in the microfluidic separation system of FIG. 33A.

FIG. 34A illustrates a microfluidic separation system utilizing multiple carrier fluid sorters in series that may be used to separate phage particles from bacteria.

FIG. 34B illustrates the bacterial and phage concentration measurements at the inlet, the retentate outlet (e.g., coupled to the sorter outlets) and the permeate outlet (e.g., coupled to the sorter side channels).

FIG. 35 illustrates bacteria, phage, and endotoxin sorting performance testing data of the microfluidic separation system of FIG. 34A.

FIG. 36A shows the separation of liposomes and 16-nucleotide molecules in an expansion region of a microfluidic separation system having a focusing channel of 5 micrometer width, 120 micrometer length and 50 micrometer height at flow rates of 40 μL/min and 80 μL/min.

FIG. 36B illustrates fluorescent images of samples of carrier fluid taken at an inlet and at different outlets in the microfluidic separation system of FIG. 36A at a flow rate of 80 μL/min.

FIG. 37A illustrates microscopic images of liposomes after sonication without membrane extrusion.

FIG. 37B illustrates microscopic images of liposomes after sonication with membrane extrusion.

FIG. 37C shows the separation of liposomes after membrane extrusion and 16-nucleotide molecules in an expansion region of a microfluidic separation system having a focusing channel of 5 micrometer width, 120 micrometer length, 50 micrometer height, and a volume split ratio of 24:1 (O2:O1) at flow rates of 40 μL/min and 60 μL/min.

FIG. 37D illustrates the intensity profiles along the A-A′ line (left) and the B-B′ line illustrated in FIG. 37C for the liposomes and 16-nucleotide molecules.

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present technology includes an inertial microfluidic separation system that is rugged and stable so that it can be moved between locations and used in the field or on site and/or that separates and/or concentrates particles in carrier fluids. The inertial microfluidic separation system may utilize the principles of inertial microfluidics to achieve separation based on particle size without requiring external forces and/or membrane filtration. The system may utilize particular configurations of microfluidic networks to leverage the differential wall lift forces to separate different sizes and/or types of particles. The system may be particularly effective for separating various biological particles including bacteria, viruses such as phage particles, extracellular vesicles, liposomes, nucleic acids, proteins, polypeptides, white blood cells, red blood cells, and platelets, as well as others.

This technology may provide significant benefits in separation of particles ranging from, for example, bacterial cells (˜0.5-2 μm) down to small nucleic acid molecules, with particular utility in phage concentration and/or separation, bacterial filtration, and isolation of cell-free DNA from liposomes or extracellular vesicles. The ability to process samples continuously without clogging may represent a substantial improvement over conventional separation methods for biological samples.

The inertial microfluidic separation system may be configured to be operated by an external power supply or batteries with significantly less power consumption than prior machines. The power savings are attributable in part due to the replacement of bulky processing equipment with a microfluidic cartridge. The microfluidic cartridge can be operated with significantly less power compared to a centrifuge or other processing equipment. Thus, the inertial microfluidic separation system may use a microfluidic cartridge for carrier fluid fractionation.

In some embodiments, the present technology includes a microfluidic cartridge that operates in an inertial microfluidic separation system. The cartridge is configured to separate and/or concentrate particles in carrier fluids. In some aspects, the inertial microfluidic separation system includes: an inlet configured to receive carrier fluid from a carrier fluid source, the carrier fluid including first particles; and a carrier fluid sorter coupled to the inlet, the sorter including a microfluidic network.

The carrier fluid sorter microfluidic network includes: a sorter constricted region having a first cross-sectional dimension; a sorter expansion region having a second cross-sectional dimension that is larger than the first cross-sectional dimension; at least one sorter side channel formed into a side of the sorter expansion region, the at least one sorter side channel configured to receive a first fraction of the carrier fluid that is first particle poor; and at least one sorter outlet that is downstream or medial from the at least one sorter side channel, that is configured to receive a second fraction of the carrier fluid that is first particle concentrated. The microfluidic network is configured to direct the first fraction of the carrier fluid to the at least one sorter side channel and configured to direct the second fraction of the carrier fluid to the at least one sorter outlet.

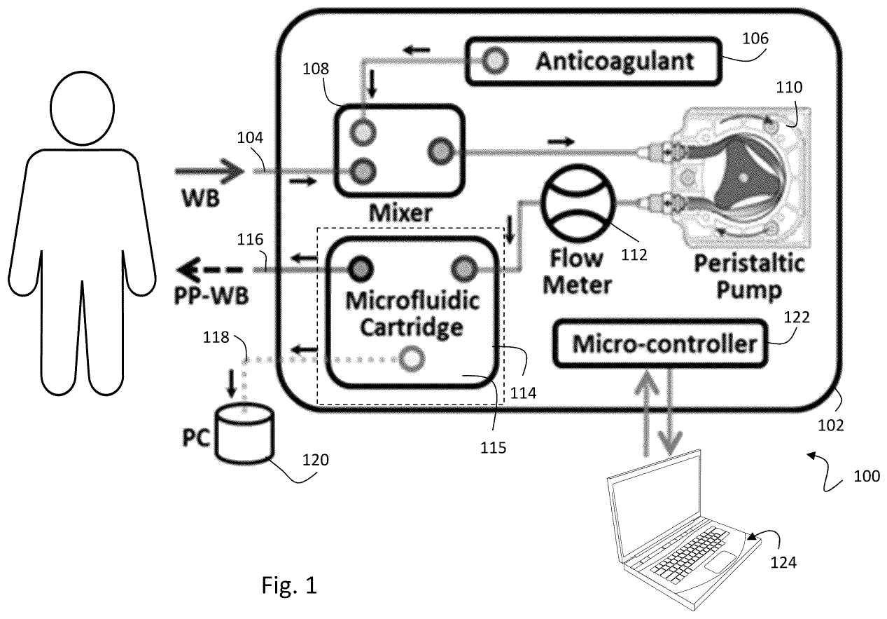

The microfluidic separation system can have various configurations. FIG. 1 illustrates an example of an embodiment of an inertial microfluidic separation system configured as a portable apheresis system. The portable apheresis system can include an outflow from a donor into the portable platelet apheresis device 100, which includes a housing 102 retaining an inlet 104, anticoagulant source 106, mixer 108 configured to mix the blood from the inlet 104 with the anticoagulant from the anticoagulant source 106, peristaltic pump 110, flow meter 112, and microfluidic cartridge 114 (e.g., removable, such as through a port 115) that includes an outlet 116 to the donor in-flow and an outlet 118 to the platelet collector 120 (“PC”). Also, a microcontroller 122 is shown, which can be connected to a computer 124 or any input/output device. In some aspects, the microcontroller 122 is a computer. While blood-based biological particles are used as an example in FIG. 1, it should be recognized that the inertial microfluidic separation system can be applied to any biological particle, as well as non-biological particles.

In some embodiments, the inertial microfluidic separation system omits or is otherwise devoid of a centrifuge or Latham bowl, or other mechanical separation device that applies a mechanical force for separation of blood components or other biological particles.

In some embodiments, the inertial microfluidic separation system can include at least one microfluidic channel network having a main channel with an inlet and a conduit between at least two outlets, where at least one outlet is a side channel outlet extending from a side wall of a main channel. The microfluidic channel network can be configured as illustrated in the figures or as described herein. The microfluidic channel network can be included in a cartridge, which can be configured to be removable and disposable as a single use consumable. The microfluidic channel network can receive undiluted blood combined with a small amount of anticoagulant, which is processed in the microfluidic cartridge in a high-throughput and high processing capacity. For example, the microfluidic cartridge can process blood at a volume flow rate about 20 mL/min, or from about 15 mL/min to about 25 mL/min, or from about 10 mL/min to about 50 mL/min, or other configurable volume flow rates as needed or desired. The length of the conduit between the inlet and at least first outlet can be modified as needed or desired.

In some embodiments, the microfluidic cartridge can be configured to be connected and interfaced with a closed loop fluidic operation system. An example of such a closed loop fluidic operation system is shown in FIG. 1. The volume flow rate can be driven with a peristaltic pump (FIG. 1) or a syringe pump, continuous pump, or any other pump, such as those capable of closed loop operation.

In some embodiments, the microfluidic cartridge consumable can include a whole blood (WB) sorter component and a platelet (PLT) concentrator component. The WB sorter and PLT concentrator can include microfluidic channels or networks housed in a cartridge housing, each with an inlet and at least two outlets. However, these components can be adapted for other biological particles.

In some embodiments, the inertial microfluidic separation system is embodied as a platelet apheresis device 100 that may be configured as a particle separation and/or concentration device that may separate and/or concentrate particles such as biological particles (e.g., phages, bacteria, exosomes, or other biological particles) included in carrier fluid. Biological particles may include naturally derived particles and synthetically derived biomimetic particles such as synthetic liposomes. In these and other embodiments, the carrier fluid may be whole blood, Luria-Bertani (LB) media, plasma, or other suitable carrier fluids. In these and other embodiments, the microfluidic cartridge consumable can include a carrier fluid sorter component and/or a particle concentrator component.

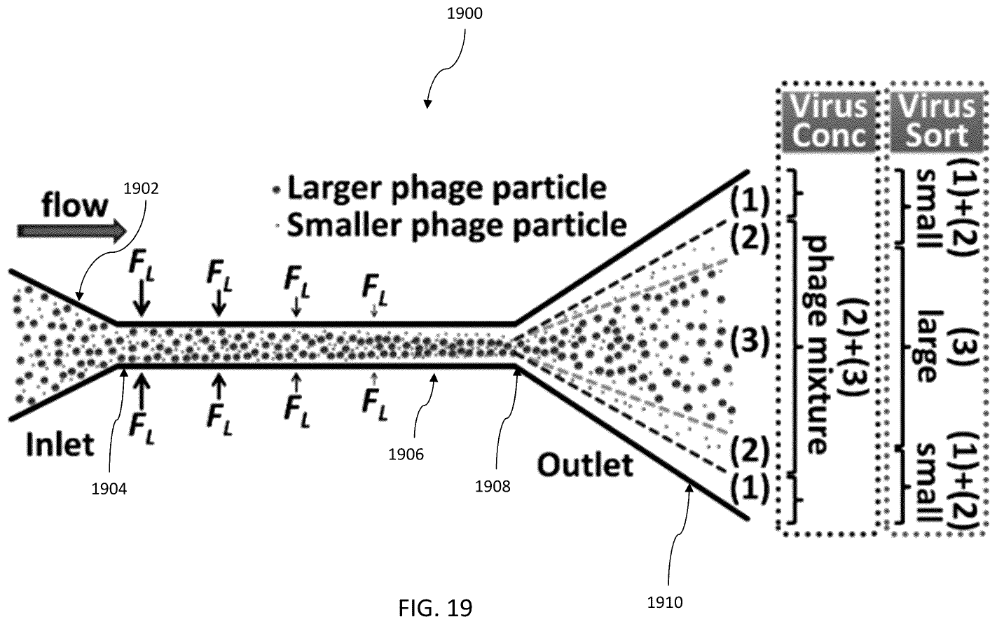

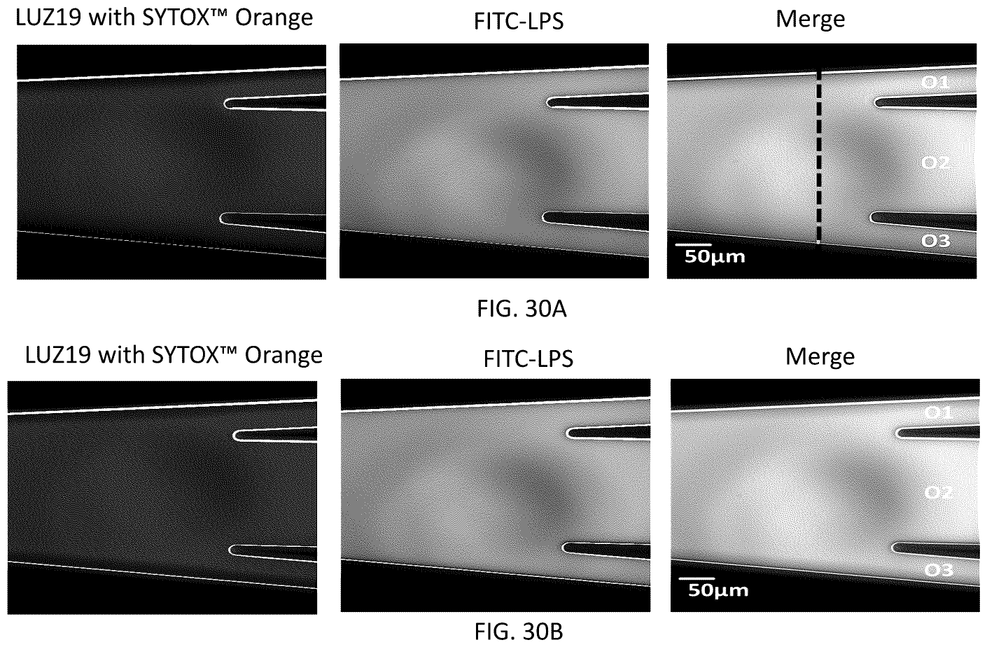

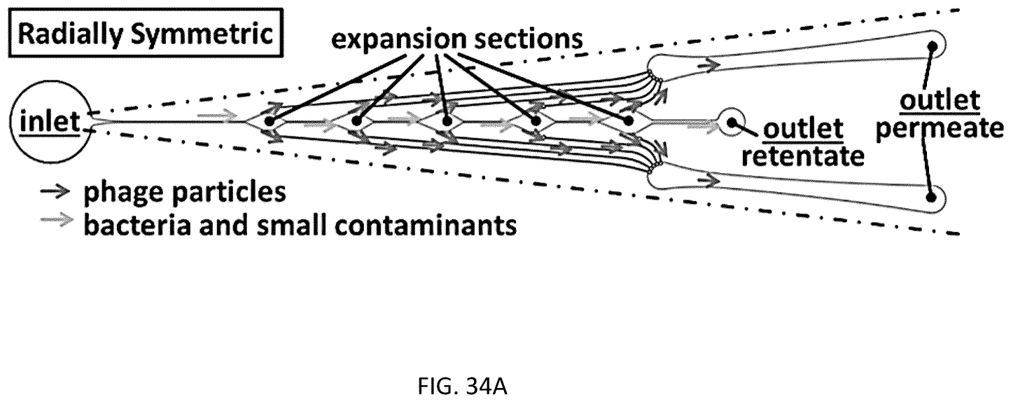

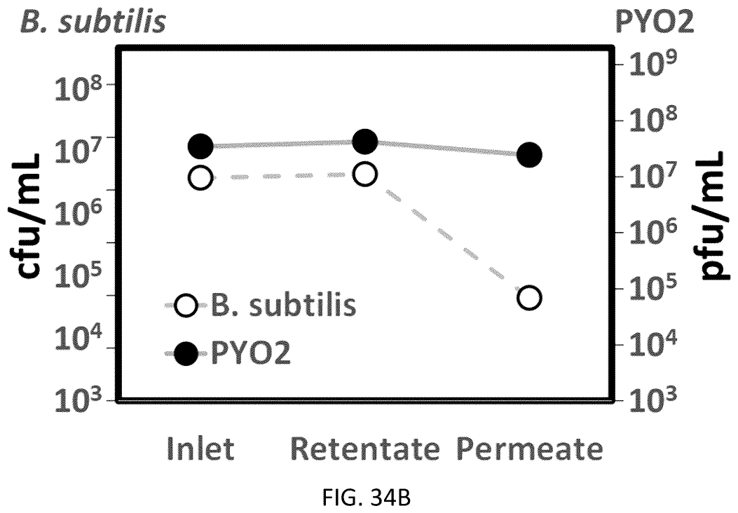

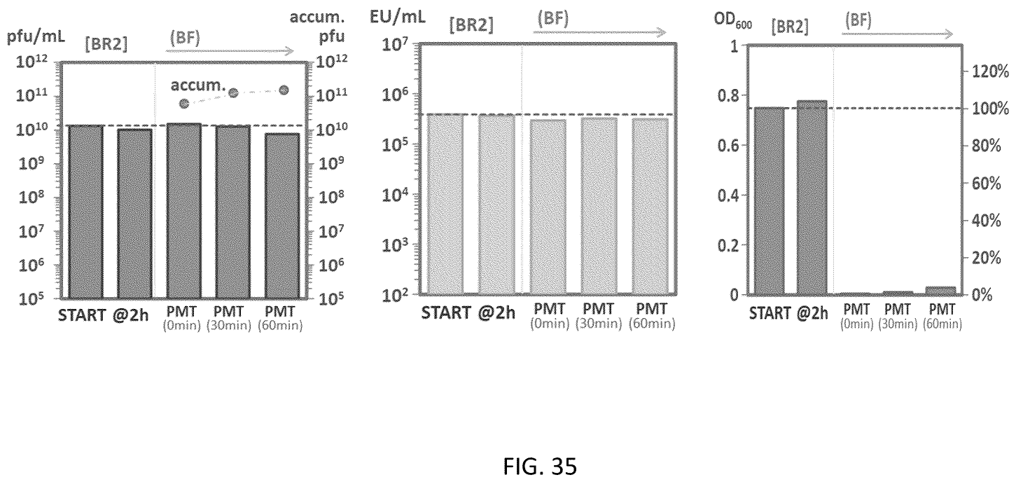

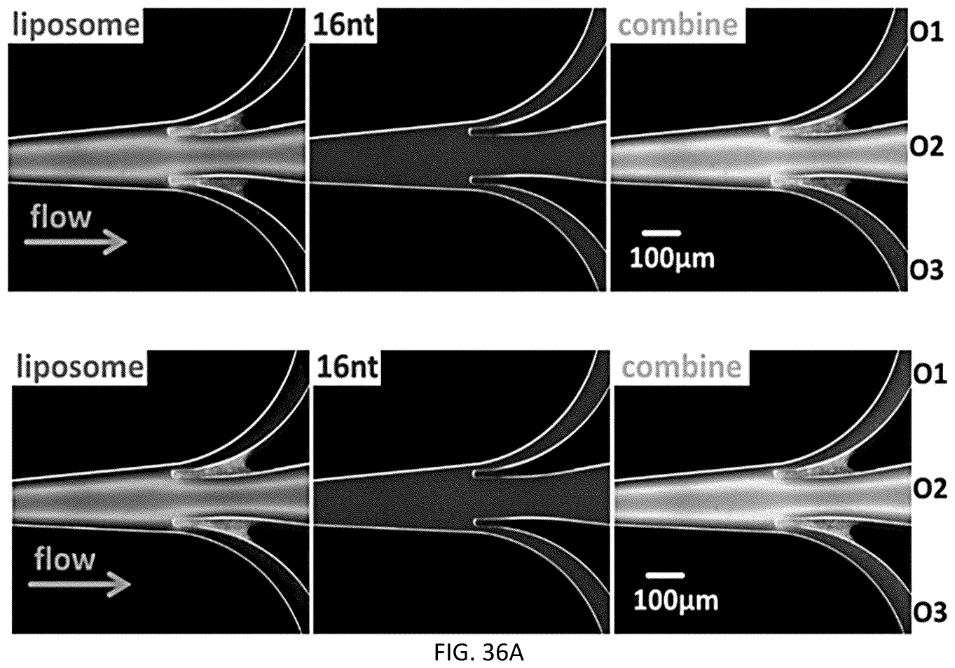

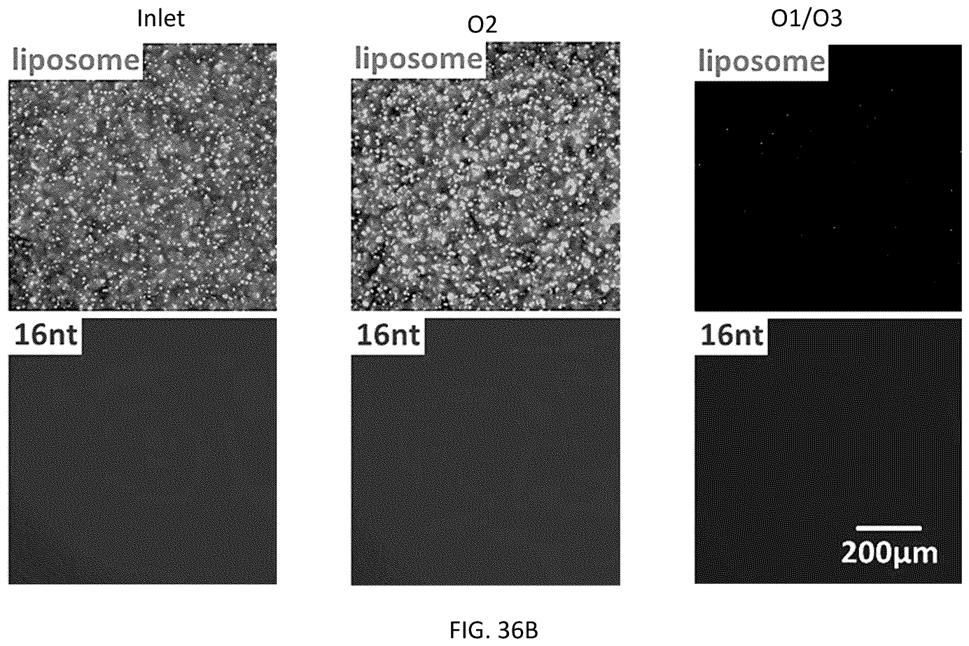

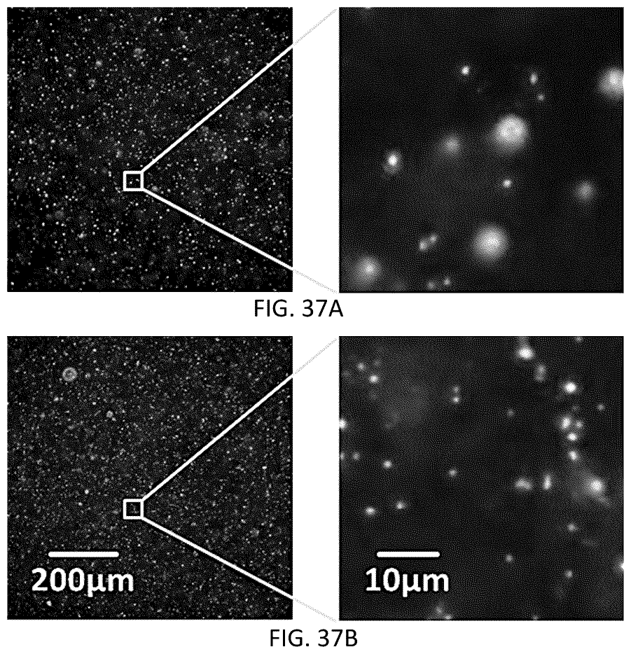

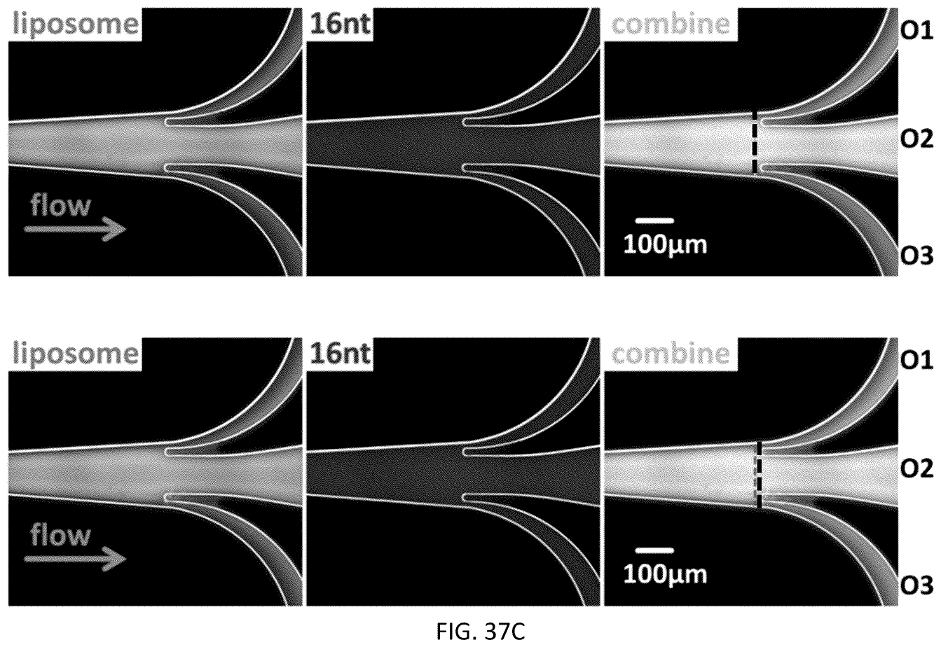

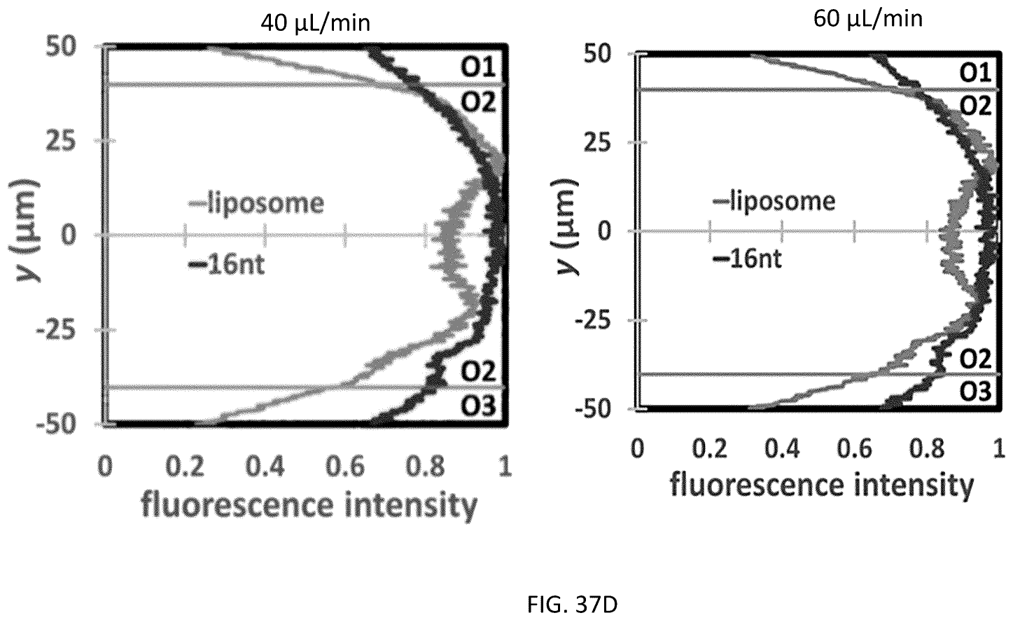

In these and other embodiments, the microfluidic networks may be configured differently based on the particles to be separated and/or concentrated. For example, the conduit between the inlet and the at least two outlets may be configured differently depending on the particles to be separated and/or concentrated. For instance, the conduit may be configured to concentrate phage particles and/or separate differently sized phage particles as illustrated in FIG. 19, the conduit may be configured to separate phage particles from bacteria in LB media and concentrate bacteria in LB media as illustrated in FIGS. 34A-35, the conduit may be configured to separate endotoxins from phages as illustrated in FIGS. 30A-32C, or the conduit may be configured to extract cell-free DNAs (16-nucleotide) from liposomes as illustrated in FIGS. 36A-37D.

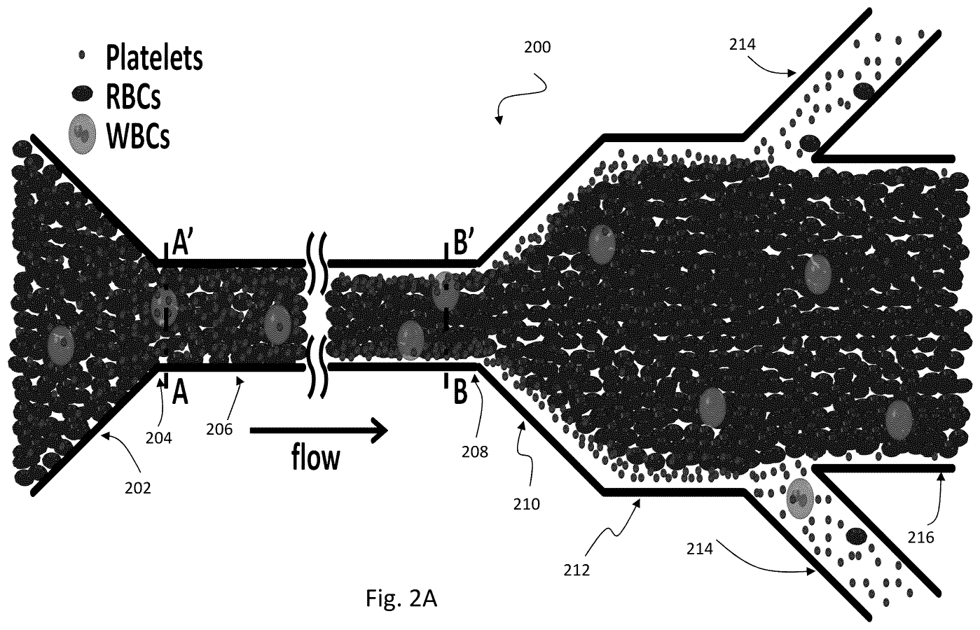

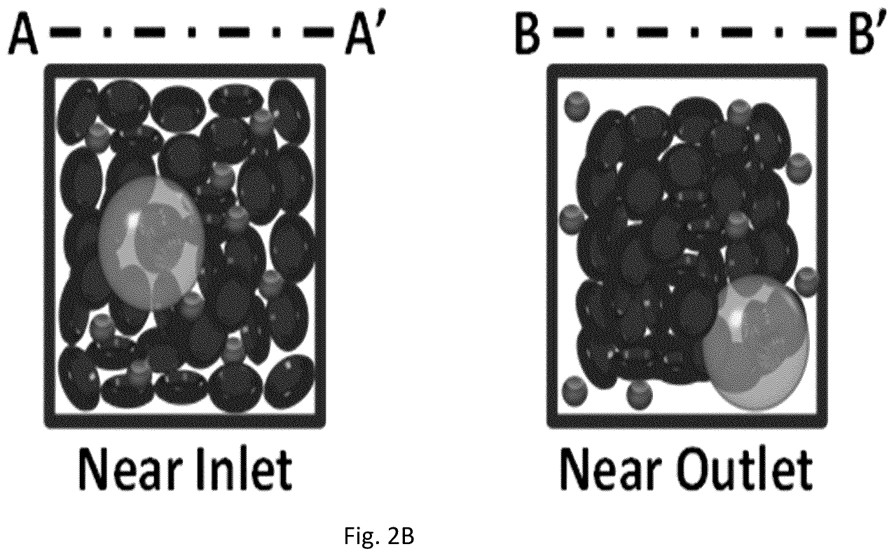

FIGS. 2A-2B illustrates a schematic illustration of a platelet separator channel 200 to provide a demonstration of platelet margination of whole blood in a constricted microfluidic channel. However, the separator channel can be adapted for separation of other biological particles. FIG. 2A is a top view or longitudinally oriented cross-sectional view of the platelet separator channel 200. FIG. 2B includes two cross-sectional views (laterally oriented) of the platelet separator channel 200 at A-A′ and B-B′. The platelet separator channel includes a narrowing tapered region 202 having a narrowed outlet 204 fluidly coupled to and/or includes of a constricted region 206. The constricted region 206 extends to a constricted outlet 208 that is fluidly coupled with an expansion region 210, which expands the dimension of the channel. The expansion region 210 is shown to have an expanded region 212 extending therefrom. The expanded region 212 includes at least one side channel outlet 214, where two side channel outlets 214 are shown, but more may be included. However, the side channel outlets may be included in the expansion inverse tapered region 210. The expansion region 212 includes at least one downstream outlet 216 downstream from the at least one side channel outlet 214, which at least one downstream outlet 216 can have the dimension of the expanded region 212 or divided into a plurality of downstream outlets. For example, the downstream outlets can be at the centerline axis as the main channel outlet.

The WB sorter unit can include a straight constricted microfluidic channel followed by an expansion microfluidic channel having at least two exits. The microfluidic channels can have various cross-sectional dimensions at the constricted region and the expansion region as shown in FIG. 2A. For example, the constricted region can include a cross-sectional dimension of at least about 20 microns, or from about 20 microns to about 100 microns, but could be larger if needed or desired (e.g., 200, 300, or up to 500 microns). The expansion region can be at least 1.2 times larger than the constricted region, or at least about 1.5 times, or at least about 2 times, or at least about 3 times, or at least about 4 times larger than the constricted region. The constricted region can be a dimension of tens of microns, and an expansion feature is located close to the channel outlet. During use, the undiluted whole blood (WB) is injected into the constricted microfluidic channel and passed through the expansion region. The smaller and rigid PLTs (e.g., 2-3 μm) are preferentially distributed near the lumen surface (e.g., marginated or moved to the margins) to form a cell-free layer (CFL) near the side walls of the constricted region, which can be due to the intense interaction with larger deformable RBCs (e.g., 5-7 μm). FIGS. 2A-2B show the smaller PLTs in a random distribution throughout the cross-section near the inlet and then preferentially distributed at the lumen surface.

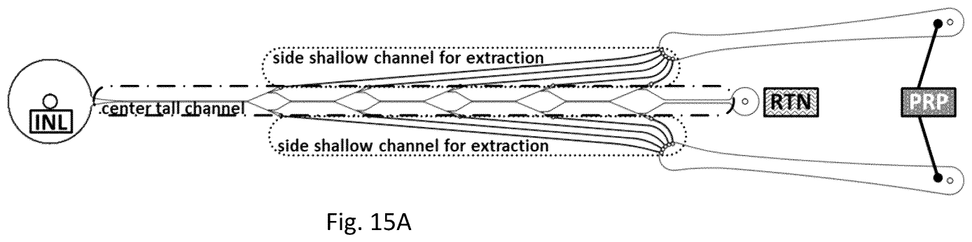

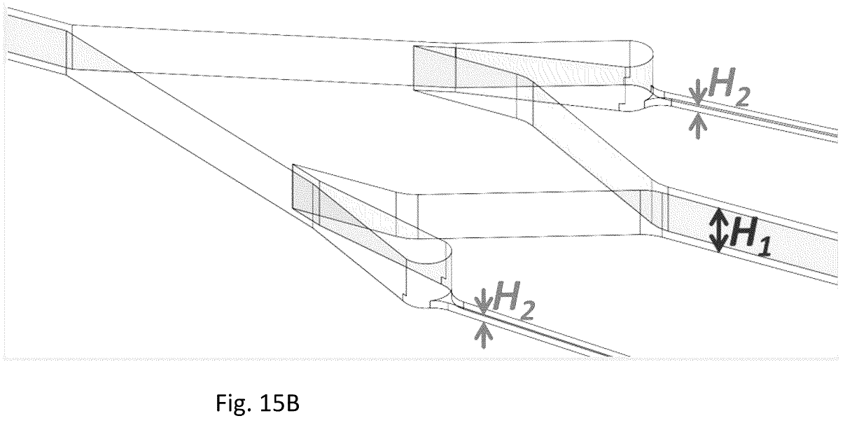

In some embodiments, the constricted region 206 may have a width between about 10 micrometers and about 60 micrometers. In these and other embodiments, the constricted region 206 may have a length between about 0.5 millimeters and about 10 millimeters. In these and other embodiments, the constricted region 206 may have a height between about 50 micrometers and about 150 micrometers. In some embodiments, the width of the constricted region 206 may be between about 10 micrometers and about 30 micrometers, the length of the constricted region 206 may be between about 0.5 millimeters and about 30 millimeters, and/or the height may be between about 25 micrometers and about 500 micrometers. In some embodiments, and as described in more detail with reference to FIG. 15B, the height of the side channel outlets 214 may be less than the height of the downstream outlet 216 such that the fluidic resistance in the side channel outlets 214 may direct a higher volume of the whole blood to the downstream outlet 216.

In some embodiments, the separator channel 200 may be dimensioned differently to separate or concentrate particles sized differently than platelets included the same or different carrier fluids. For example, the constricted region 206, the expansion region 212, the side channel outlets 214, and/or the downstream outlet 216 may configured to separate other particles such as phage particles, bacteria, exosomes, or other biological particles.

As an example, the constricted region 206 may be configured with different widths, lengths, and/or heights depending on the particular particle size or type to be separated or concentrated in the carrier fluid. As another example, the angle of the expansion region 210 relative to the constricted region 210 may be varied depending on particle size or type, the number of side channel outlets 214 and/or downstream outlets 216 may be varied depending on particle size or type, and/or the heights of the side channel outlets and/or downstream outlet 216 may be varied depending on particle size or type.

FIG. 2B shows the distribution of the platelets (PLTs), red blood cells (RBCs), and white blood cells (WBCs) at the inlet (near inlet) and outlet (near outlet) of the constriction region. In the expansion region, the PLTs are preferentially dispersed to the outside of the channel so as to be near the lumen surface. This allows for marginated PLTs to be collected by two side PRP (platelet-rich plasma) side channel outlets, as shown in FIG. 2A. However, it should be recognized that only one PRP side channel may be used, or any number of side channels can extend from the expansion region. Most RBCs migrate towards the channel axial centerline. Accordingly, the RBCs are collected by the center outlet (e.g., return outlet) and then routed to return to the blood donor. Additionally, most WBCs (e.g., 7-15 μm) are collected in the center outlet channel and returned to the donor.

The constricted region of the microfluidic channel can be considered to be an inlet into the WB sorter unit. The expansion region and the PRP side channel outlets (e.g., channels that are outlets from the expansion region) can be upstream from and fluidly coupled to a PLT concentrator unit. As such, the PLT concentrator unit is downstream from the WB sorter unit.

In some embodiments, to increase PLT extraction/collection efficiency, several constriction channel/expansion features can be arranged in serial configuration in the WB sorter. Optimized sorting and recovery performance can be achieved by balancing the fluidic resistance at each “PRP” side channel outlet and each “return” outlet channel.

In some embodiments, one or more PLT concentrators are connected directly with each WB sorter unit in serial configuration, from the sorter side channels. To increase the throughput rate of the microfluidic cartridge for platelet apheresis, typically on the order of 20 mL/min to 100 mL/min, multiple serial WB sorter channel networks and PLT concentrator channels can be arranged in parallel, such as in a radial configuration. Each radial WB sorter channel network and PLT concentrator channel network can be configured as plates that can be stacked up to further increase the throughput. As noted, each concentrator inlet is coupled to a sorter side channel to obtain the platelets.

In some embodiments, the microfluidic cartridge (e.g., consumable cartridge) can be fabricated with injection molding. The microfluidic cartridge can be configured as microfluidic disk, which can be made of medical-grade cyclic olefin copolymer (COC) or any other polymer materials and manufactured using direct laser writing mastering in combination with variothermal (localized time-dependent temperature control) injection-compression molding.

In some embodiments, the inlets and/or outlets of the microfluidic network can be fabricated on the lid layer of the cartridge, and laser welded with the molded COC on-chip-cassette. Disposable tubing and fluidic connections of the microfluidic cartridge can be fitted to various microfluidic sub-components of the automated processing apparatus.

### Wb Sorter Unit (Single Pass)

As indicated above, FIGS. 2A-2B illustrates a schematic illustration of platelet separator channel 200 to provide a demonstration of platelet margination of whole blood in a constricted microfluidic channel. FIG. 2A is a top view or longitudinally oriented cross-sectional view of the platelet separator channel 200. FIG. 2B is cross-sectional view (laterally oriented) of the platelet separator channel 200. For example, the downstream outlets can be at the centerline axis as the main channel outlet.

In some embodiments, the microfluidic network can have a high aspect ratio (HAR) straight microfluidic channel, which can be used for PLT margination for WB samples. The microfluidic device can include a small focusing channel (e.g., 8 mm×20 μm×50 μm (L×W×H)), which can have a 30° gradual expansion (e.g., range can be from 30 degrees expansion to 90 degrees expansion) collection channel, and at least one side channel outlet (e.g., 9 side channel outlets). Each side channel outlet can include a cross-dimension of about 100 μm in width.

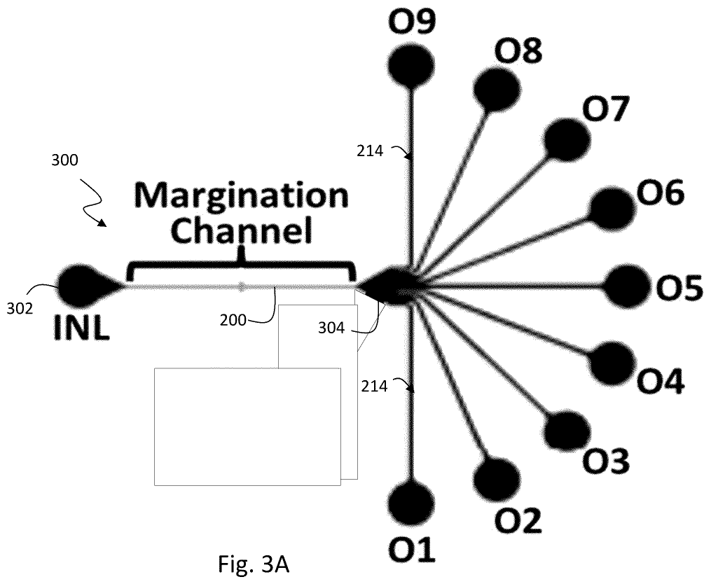

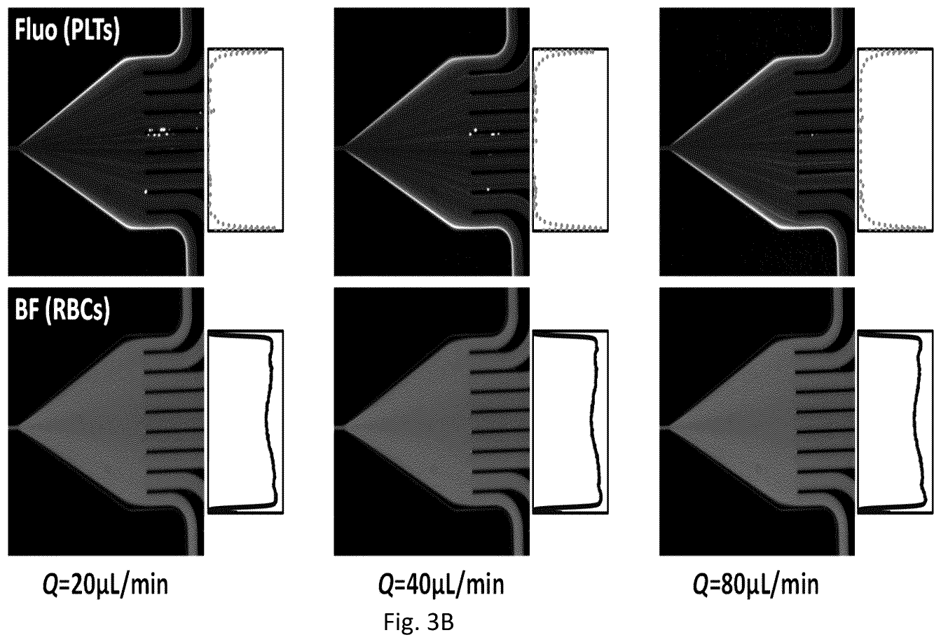

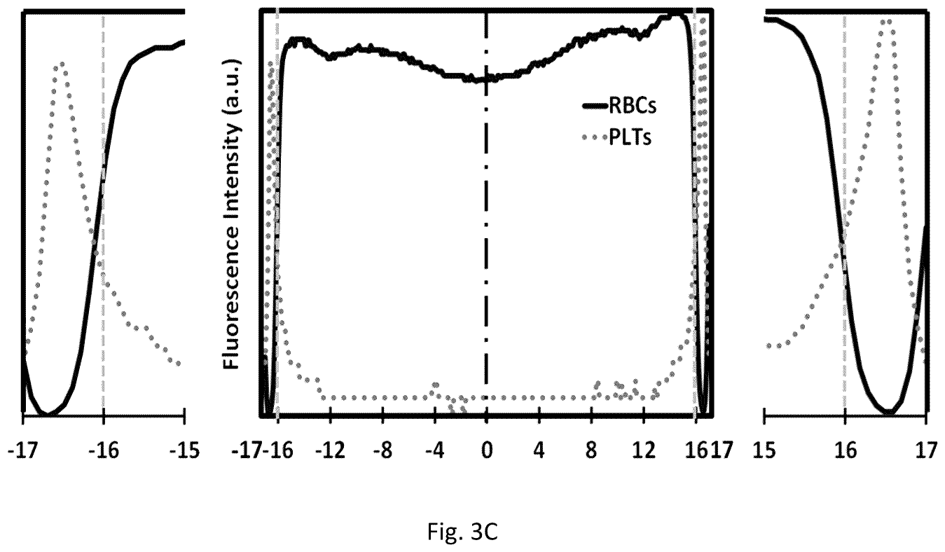

FIG. 3A shows an embodiment of a microfluidic network 300 having the inlet 302 (INL), platelet margination channel 200 with the outlet expansion channel 304 (e.g., expanded region 212) with a plurality of side channels 214 (e.g., side channels O1, O2, O3, O4, O5, O6, O7, O8, and O9); however, more or less side channels may be used.

To enable visualization of PLT migration, purified and fluorescently labeled human PLTs with Calcein AM were prepared. The Calcein AM stained PLT pellet was re-suspended in original PPP and mixed with stored RBCs to replicate the initial whole blood cell counts and concentration. FIG. 3B shows the margination of Calcein AM stained PLTs in the expansion channel at flow rate ranging from 20 μL/min to 80 μL/min. At flow rate of 20 μL/min (Re˜3), RBCs experience high shear and inertia in the focusing channel which facilitate their migration to the channel centerline, while the smaller and rigid PLTs are marginated towards the channel side walls. In the outlet expansion channel, two tightly marginated PLT flow trajectories formed in proximity with the expanded CFL region were collected in outlets O1 and O9 as shown in FIG. 3A. Due to their high concentration, the RBCs trajectories spanned across outlets O1-O9, leaving a small portion of CFL region near the side wall of outlets O1 and O9. The fluorescence and bright field intensity profiles were plotted next to the experiment flow trajectories images at each flow rate. When increasing the flow rate up to 200 μL/min (Re˜31), similar flow trajectories and intensity profiles were observed at different flow rates.

The microfluidic network 300 may be configured as discussed previously to separate and/or concentrate other types and/or sizes of biological particles. For example, the platelet margination channel 200 may be configured with different dimensions (e.g., length, width, and/or height) to separate and/or concentrate other biological particles in the same or different carrier fluids. This is discussed in more detail with reference to FIGS. 19-37C.

FIGS. 3A-3C demonstrate PLT margination in WB samples with a high aspect ratio (HAR) straight microfluidic channel. FIG. 3A shows a schematic of the system. FIG. 3B shows the PLT collection at different flow rates (Fluo-Fluorescent imaging of stained PLTs; BF—Bright-field imaging of RBCs). FIG. 3C shows intensity profiles at 80 μL/min (Dotted line—Intensity profile for PLTs acquired using fluorescence imaging; Solid Line—Intensity profile for RBCs acquired using bright-field imaging).

To demonstrate and characterize performance in WB sorting and PLT extraction in constricted the HAR microfluidic channel using samples collected “off-chip”, outlet fluidic resistance of the high aspect ratio straight channel device is manipulated to optimize for RBC and PLT recoveries for high PLT purity. Examination of the fluorescence (PLTs) and bright field (RBCs) intensity profiles at volume flow rate of Q=80 μL/min, found that the optimal volume flow ratio between outlets “PRP” (outlets O1 and O9) and “RBC Return” (outlets O2-O8) recovery is about 1:17 (FIG. 3C), which allows a considerable amount of PLTs to be extracted in outlets O1 and O9, and a majority of RBCs (>99.9%) to be recovered in the “return” outlet (outlets O2-O8).

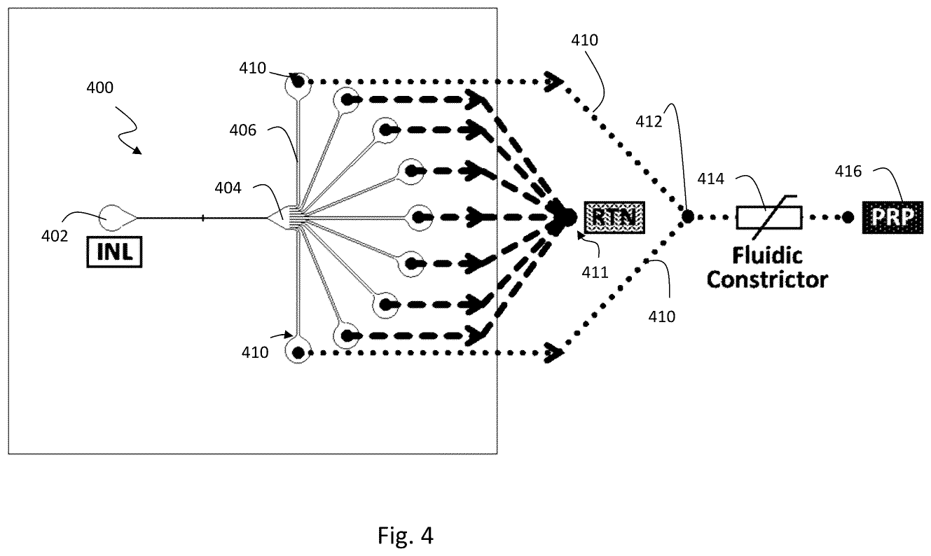

Additionally, the system can be formed to include the HAR microfluidic device coupled with other microfluidic devices that allow for achieving the desired fluidic resistance necessary for optimal separation of platelets and RBCs (see FIG. 4). The outlets O2-O8 of the high aspect ratio (HAR) straight microfluidic channel were grouped together as a return (e.g., RTN 411) to the patient (e.g., to patient inflow, outlet 116). The outlets for the PRP (e.g., outlets O1 and O9) of the HAR microfluidic channel were combined and connected to microfluidic devices (e.g., 1.5 cm dimension) connected to two inlets (e.g., 410) of a bifurcation channel (BIF) device. In an example, the BIF microfluidic device includes one parent 412 and two daughter channels 410. In an example, the width of parent channel and daughter channels are 100 μm and 50 μm respectively, but can vary (e.g., 5%, 25%, 50%). Both parent and daughter channels can have a constant height (e.g., 50 μm). The outlet of the BIF microfluidic channel (e.g., parent 412) can be connected to the inlet of the linear channel (LC) microfluidic device. However, one, two, or more than three straight channels can be used. By connecting the microfluidic devices in series, the expected fluidic resistance between “PRP” and “Return” outlets was achieved as 1:17.5.

FIG. 4 shows the schematic of the WB sorter 400 for PLT extraction with HAR straight microfluidic channel. PRP outlets were connected to 3× linear channels with length of 1.5 cm each and cross-section of 100 μm×100 μm. This microfluidic assembly is constructed to manipulate for fluidic resistance to optimize for RBC and PLT recoveries. In another example, freshly acquired WB sample is injected into the microfluidic module (FIG. 4) at a volume flow rate of 80 μL/min.

FIG. 4 shows the WB sorter 400 includes the inlet 402 (INL) that feeds into a separator region 404 that has the arranged outlet lines 406 that correspond with outlets O1 through O9 (as defined herein). The outlet lines 406 of O1 and O9 outlets go to a mixer 412 that feeds into a fluidic constrictor 414 to obtain the platelet rich portion 416.

The WB sorter 400 may be configured as discussed previously to separate and/or concentrate other types and/or sizes of biological particles in the same or different carrier fluids. This is discussed in more detail with reference to FIGS. 19-37C.

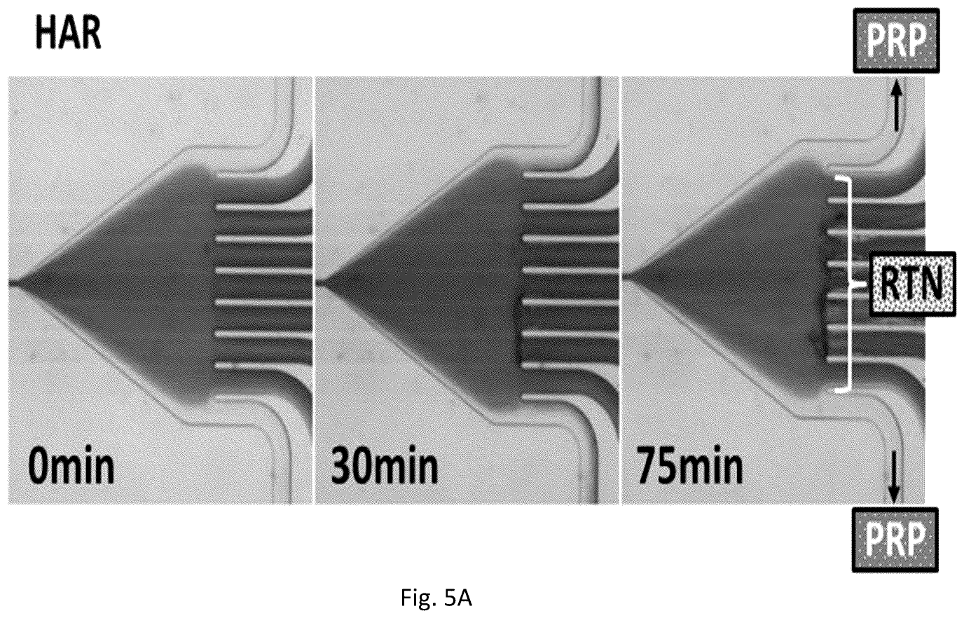

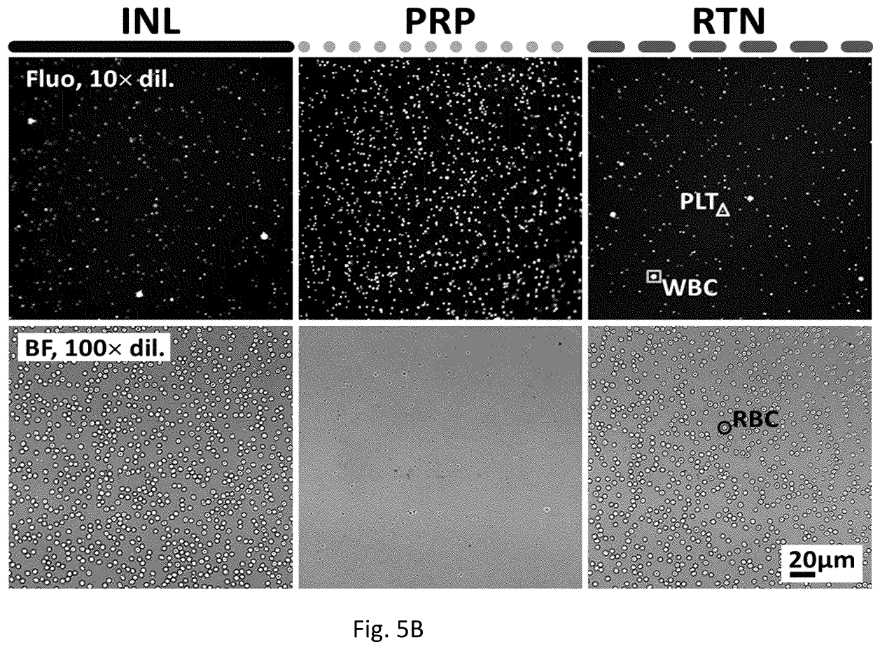

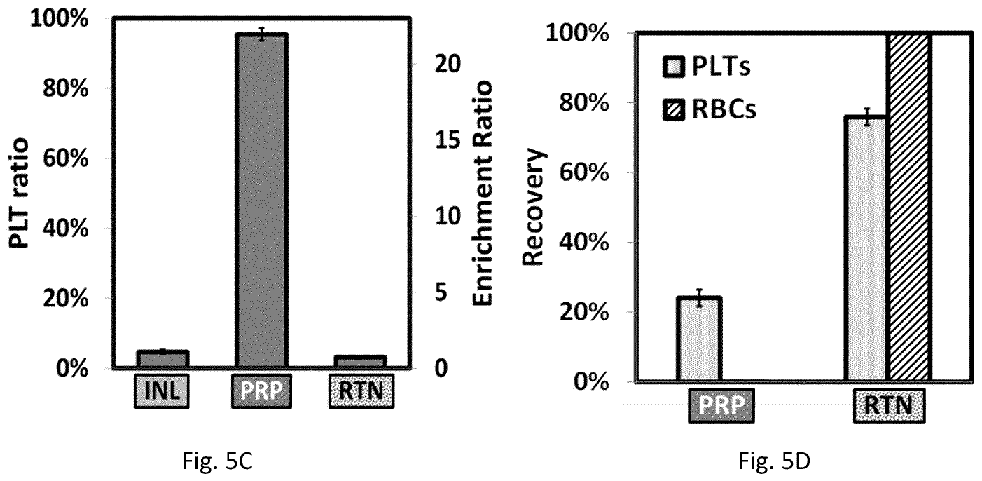

In the example shown in FIG. 5A, the flow trajectory of inertial focusing is shown in the expansion channel under bright field imaging. PLT-depleted sample were collected from outlets O2 to 08 as “Return”. RBC-depleted and PLT-enriched PRP samples are collected in outlets O1 and O9 “PRP” outlets. On average, about 4 mL of WB was processed with single set of microfluidic devices in single pass without clogging at processing time of about 1 hour.

FIGS. 5A-5D demonstrate WB sorting with the HAR rectangular straight microfluidic channel assembly at volume flow rate of Q=80 μL/min with undiluted WB: FIG. 5A shows the inertial focusing of RBCs and WBCs in the expansion channel closed to the outlet; FIG. 5B shows the cell count (Square: WBC; Triangle: PLT, and Circle: RBC) counting under cell counting chamber; FIG. 5C shows the PLT ratio and enrichment plots; and FIG. 5D shows the recovery plot.

The purity, recovery and enrichment ratio were determined as the follows:

$$Purity [ PLT ] = \frac{[ {C}_{PLT} ]}{[ {C}_{PLT} ] + [ {C}_{RBC} ] + [ {C}_{WBC} ]} \qquad [ 1 ]$$

$${Recov [ RBC ]}_{Return} = \frac{{[ {N}_{RBC} ]}_{Return}}{{[ {N}_{RBC} ]}_{PRP} + {[ {N}_{RBC} ]}_{Return}} \qquad [ 2 - a ]$$

$${Recov [ PLT ]}_{PRP} = \frac{{[ {N}_{PLT} ]}_{PRP}}{{[ {N}_{PLT} ]}_{PRP} + {[ {N}_{PLT} ]}_{Return}} \qquad [ 2 - b ]$$

$${Enrich [ PLT ]}_{PRP} = \frac{{Purity [ {C}_{PLT} ]}_{PRP}}{{Purity [ {C}_{PLT} ]}_{Inlet}} \qquad [ 3 ]$$

where [N_{PLT}], [N_{RBC}] & [N_{WBC}] are the number of PLTs, RBCs and WBCs collected from the inlet or outlets, while [C_{PLT}], [C_{RBC}] & [C_{WBC}] are the concentration of PLTs, RBCs and WBCs from the inlet or outlets respectively.

The cell samples collected from inlet, outlet “PRP” and “Return” were counted with bright field and fluorescence imaging in FIG. 5B. The RBCs (circle) portion of all samples was counted in bright field imaging with 100× dilution in 10% ACD 1×PBS. The WBCs and PLTs portion were counted with 10× dilution and Calcein AM staining in fluorescence images. Cells with >4 μm diameter were counted as WBCs (square), while cells with diameter <4 μm were counted as PLTs (triangle). The sorting performance was summarized in FIGS. 5C and 5D. For single pass operation, the microfluidic module recovered 99.95% of RBCs. The PLT ratio in the “PRP” collection was 95.4%, which is enriched 21.6× from an initial PLT ratio of 4.7%. On average 24.1% of PLTs were extracted in the “PRP” collection. WBC concentration is reduced ˜7× in the outlet “PRP” compared to the inlet. On average, less than 0.05% of RBCs were collected in the outlet “PRP”.

### PLT Concentrator Unit

In some embodiments, the focusing region of the PLT concentrator unit can include a straight high aspect ratio (HAR) channel (e.g., height>width) that has more height than width. For example, the PRP sample collected from the WB sorter is injected to the PLT concentrator. The PLTs forms two focused trajectories as they traverse the HAR focusing channel into the expansion outlet channel of the PLT concentrator, the PLT focusing streams are somewhat towards the two side walls or generally away from the true center with plasma substantially devoid of PLTs adjacent to the side walls. The PLT concentrator has two focused PLT streams that are not along the walls, and thereby the function of the PLT concentrator is different from the WB sorter. Even the regions more centrally to the PLT focusing streams have less PLT than the focusing streams, but more PLT than the outer side wall streams of plasma. The PLT content of the PRP sample can be concentrated and collected in the side channel outlets of the concentrator due to the PLT trajectory, which can be formed into and extend from the sides, top, or bottom of the main channel, or from respective side channels when circular or elliptical cross-sectional profile. The PLT concentrator can be configured similarly to the WB sorter, and located downstream from the WB sorter. That is, FIG. 2A can represent the structure of the PLT concentrator. The difference is the outlet in the concentrator that receive the concentrated platelet fraction is not the outer channels or side channels, but the concentrated platelet fraction are the channels inward or medial or downstream from the outer channels or side channels. In the concentrator, the side channels (e.g., ultimate from central axis) are for a platelet poor plasma (PPP) portion. The next inward channels (e.g., penultimate from central axis) collect the platelet concentrate. Then, the next inward channels (e.g., medial to antepenultimate) collect platelets but at lower concentration than the platelet concentrate.

In some embodiments, a leukocyte reduction filtering unit can be included in the microfluidic system downstream or after the PLT concentrator unit to decrease the WBC concentration in the “PC”. The leukocyte reduction filtering unit can be downstream from the opening of the side channels.

In the concentrator, the main channel or other side channels downstream from the openings of the side channels can be used to collect the other portion of the “PRP” sample. The concentrator side channels are used as “PPP” (platelet-poor plasma) outlets and then routed to return to the blood donor to prevent excess loss of plasma.

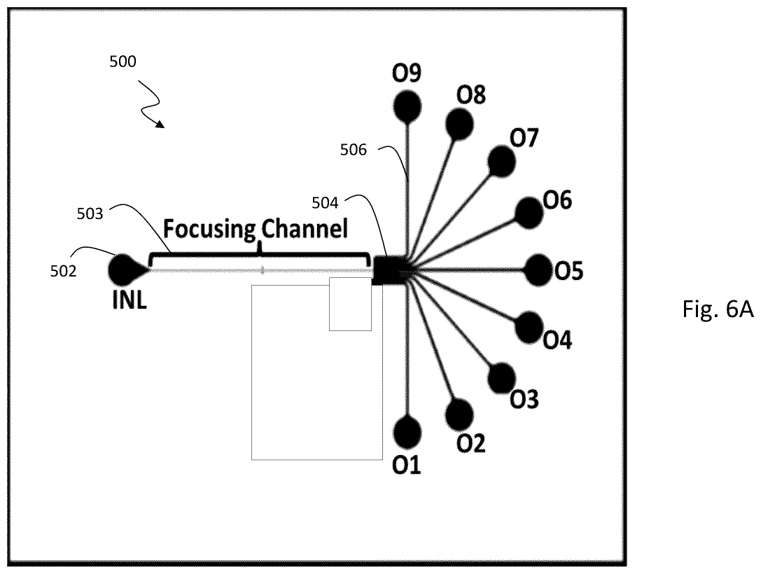

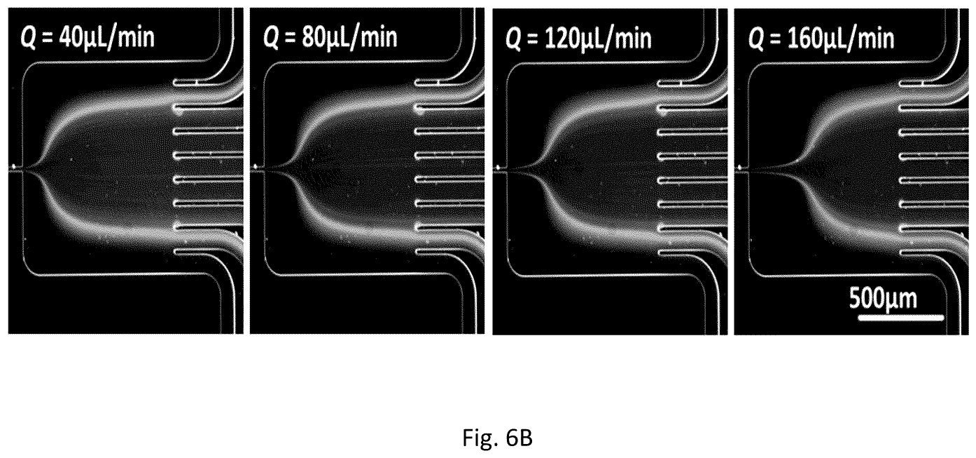

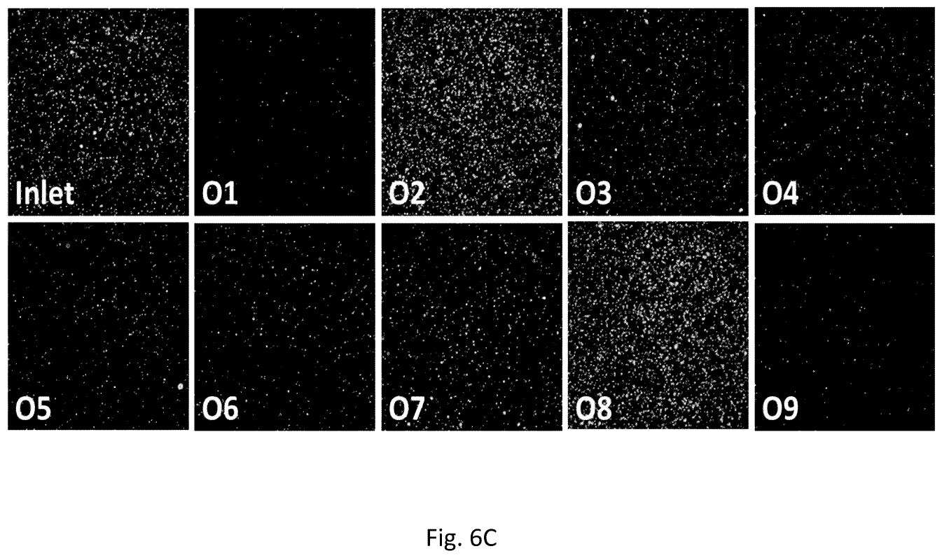

In an embodiment of a PLT concentrator unit, the fluorescently labeled human PLTs are used to characterize the performance of HAR microfluidic channel device (FIG. 6A) for PLT concentrating. As shown, the expansion region is not an inverse taper, but a full expansion into a larger conduit. Although a full 90-degree expansion channel is shown, other expansion geometries work as well.

FIG. 6A shows the PLT concentrator 500 includes the inlet 502 (INL) that feeds a focusing channel 503 into a separator region 504 (e.g., outlet expansion channel) that has the arranged outlet lines 506 that correspond with outlets O1 through O9 (as defined herein). The outlet lines 506 of O1 and O9 outlets can go to reservoirs or be used to collect the contents of each output line, or they can be used as input into a next stage, or appropriately recycled back.

The expansion point of the PLT concentrator unit is shown in FIG. 6B, which demonstrates the inertial focusing of Calcein AM stained PLTs in the sudden expansion channel (e.g., no expansion inverse taper) at flow rate ranged from 40 μL/min to 160 μL/min. Two PLT focused flow trajectories were formed near the side walls of the expansion channel. Most fluorescently labeled PLTs were directed to collection outlets O2 and O8 (e.g., platelet concentrate). Still, a good portion of platelets was collected from outlets O3 to O7. Very few PLTs flowed into outlets O1 and O9 (platelet poor plasma). In FIG. 6C, concentrated/filtrated Calcein AM stained PRP samples collected from outlets O1 to O9 were counted. The average volume flow rate was Q=120 μL/min. Accordingly, the plasma that is poor in platelets (PPP) can be collected from outlets O1 and O9.

FIGS. 6A-6C show the concentration of PRP with the high AR straight rectangular microfluidic channel device (e.g., sudden expansion or sharp expansion without an inverse taper): FIG. 6A shows the device layout with focusing channel of 8 mm length, 20 μm width and 50 μm height, and 9 outlets; FIG. 6B shows the focusing and collection of Calcein AM stained human PLTs in the expansion channel at different flow rates 40-160 μL/min; and FIG. 6C shows the collection enriched/depleted human PLTs at different outlets at average flow rate of 120 μL/min. About 65% of PLTs were collected in outlets O2 and O8, resulting in PLT concentration factor of 3×. 96% PLTs were retained from outlets O2 through O8 with only 4% PLT lost in outlets O1 and O9. In this case, a concentration factor of 1.3× resulted.

The platelet concentrator 500 may be configured as discussed previously to concentrate other types and/or sizes of biological particles. For example, the focusing channel 503 may be configured with different dimensions (e.g., length, width, and/or height) to separate and/or concentrate other biological particles in the same or different carrier fluids. This is discussed in more detail with reference to FIGS. 19-37C.

### Schematic of the Connection of Serial Wb Sorter and PLT Concentrator Units

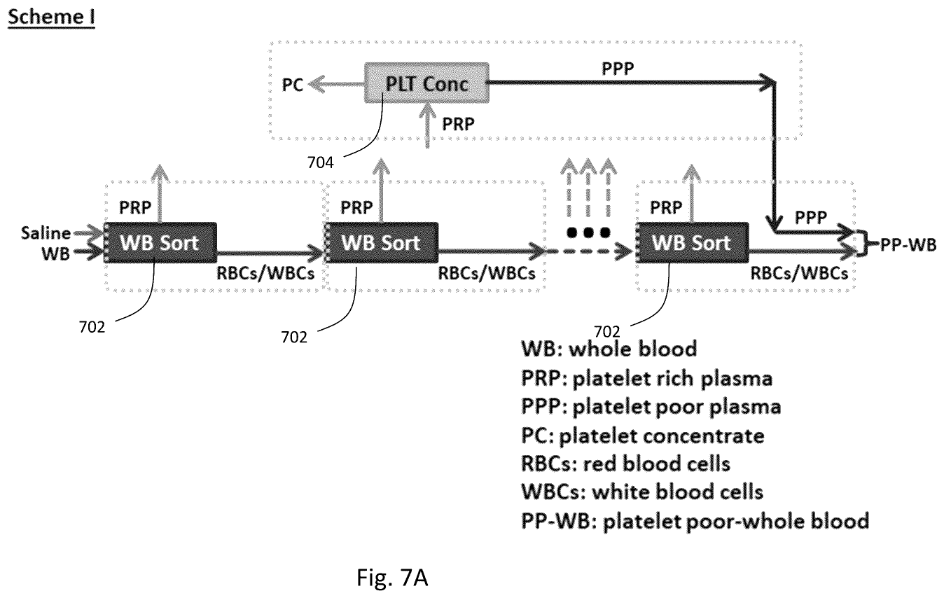

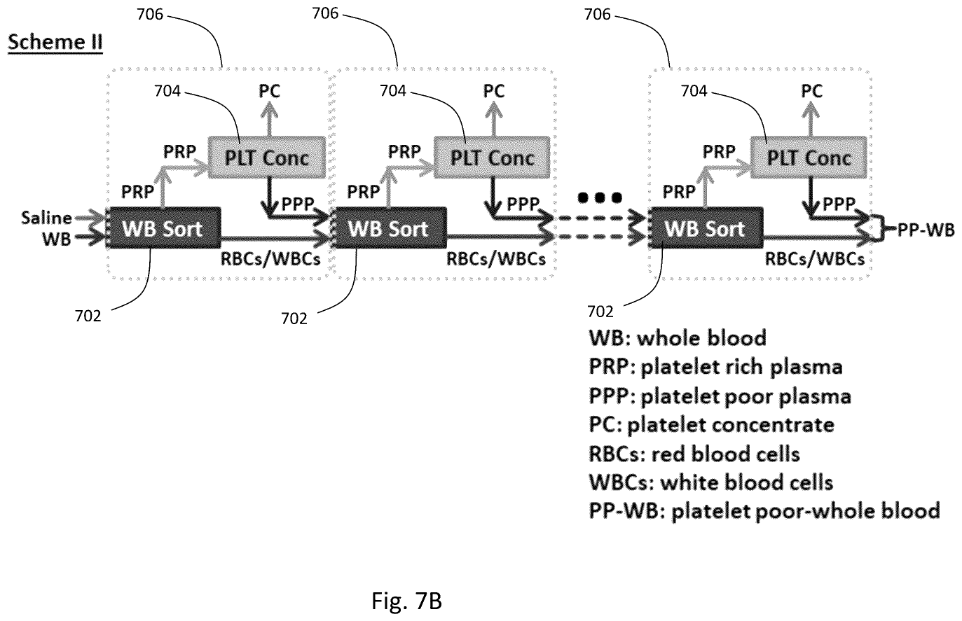

The microfluidic module with optimized outlet recovery has good sorting performance with high PLT purity in outlet “PRP” and high RBCs recovery in outlet “Return”. However, the single pass PLT extraction efficiency is low (˜25%). Higher platelet extraction efficiencies can be achieved by connecting multiple high aspect ratio (HAR) sorters in a serial manner. We identified two different schemes for collecting the HAR sorters along with platelet concentrators (PLT Conc) that will allow realization of the portable apheresis device capable of producing platelet product with desired quality (FIGS. 7A-7B). The schemes include Scheme I and Scheme II.

Scheme I: The PLTs will be continuously extracted from the serially arranged WB sorters. All PLTs extracted will be collected and concentrated using one common PLT concentrator. The advantage of this scheme is the ease of fabrication due to less fluidic interconnects. However, a single PLT concentrator will require the ability to extract platelets with a high PLT recovery efficiency. Scheme II: This arrangement requires more complicated fluidic interconnects. Each repeating unit is composed of a pair of WB sorter and PLT concentrator. Platelet concentrate (PC) will be extracted in each PLT concentrator and platelet poor plasma (PPP) will be routed to the WB sorter in the next unit. The addition of PPP to the next WB sorter will help in preventing excess loss of plasma. This approach imposes lower requirement on PLT recovery efficiency for the PLT concentrator and is expected to have higher overall recovery of PLT.

FIGS. 7A-7B show schematics of PLT apheresis device for undiluted continuous WB processing. WB sorter operates based on PLT margination effect. PLT concentrator operates based on inertial focusing of PLTs.

FIG. 7A shows Scheme I, where the PRP extracted from all WB Sorter units is collected and processed with one common PLT concentrator unit. As such, saline and whole blood (WB) are input into a whole blood sorter 702, where the platelet rich portion (PRP) is withdrawn and the RCB/WBC portion goes to a second whole blood sorter 702, which can be repeated to have “n” whole blood sorters, where n is an integer. The PRP from each WB sorter 702 is input into a platelet concentrator 704, which obtains the platelet poor plasma (PPP) and the platelet concentrate (PC).

FIG. 7B shows Scheme II, where each PLT extraction unit 706 consists of a pair of a WB sorter 702 and a PLT concentrator 704. The PPP is re-injected to the next unit (706) to improve PLT recovery and extraction efficiency.

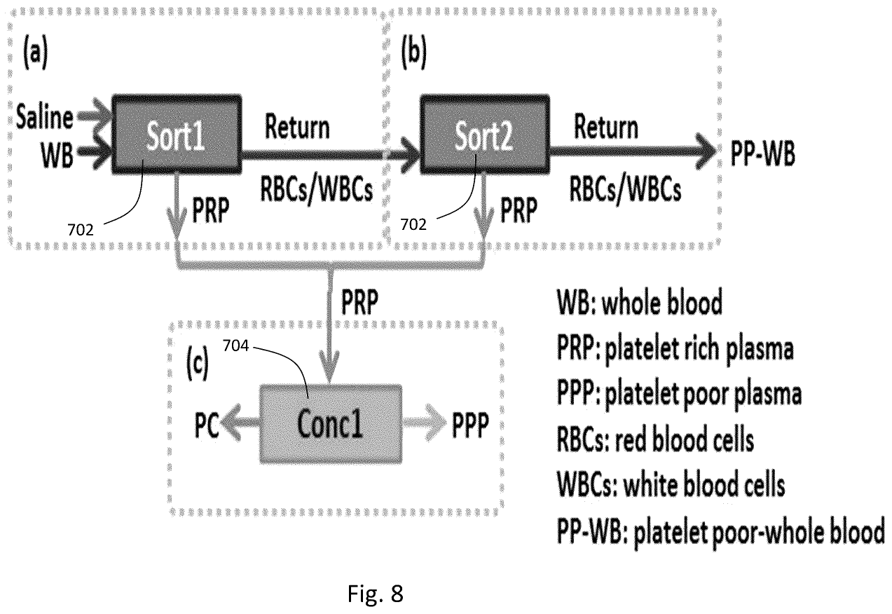

Scheme I was implemented using two WB sorters (Sort1, Sort2) and one PLT concentrator (Conc1) in batch processing operation using whole blood as input sample (FIG. 8).

FIG. 8 shows an example of serial WB sorting, PLT extraction and concentration in batch processing mode: (a) 1^{st} PLT extraction from WB Sort1 (702); (b) 2^{nd} PLT extraction from Sort2 (702); and (c) PLT concentration from PLT Conc1 (704) from both WB Sort1 and WB Sort2.

The operation parameters and experimental conditions of both Sort1 and Sort2 were similar to single pass experiment in the previous section. Both Sort1 and Sort2 feature identical microfluidic modules. The sample collected from outlet “Return” of Sort1 was re-injected to the inlet of Sort2. We found that the PLT extraction efficiency in the 2^{nd} pass was similar to 1^{st} pass, which was about 25%. The PRP collected from 1^{st} and 2^{nd} WB sorter had purity >95%. Over 50% of PLTs from WB were collected with the two sorters.

We used HAR straight microfluidic channel as Conc1. The sample collected from outlets of “PRP” of Sort1 and Sort2 were mixed together and re-injected to the inlet of Conc1. The PLT concentration was increased in 2× in the outlet “PC” of Conc1.

The WB sorters 702 and/or the PLT concentrators 704 illustrated in FIGS. 7A, 7B, and 8 may be configured as discussed previously to concentrate other types and/or sizes of biological particles in the same or different carrier fluids. This is discussed in more detail with reference to FIGS. 19-37C.

### Microfluidic Design of Serial WB Sorters and PLT Concentrator Unit in Continuous Operation Mode

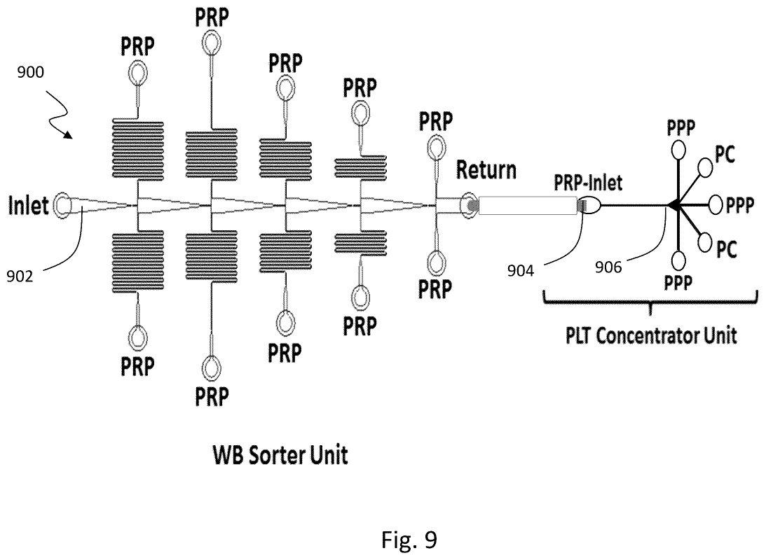

Multiple WB sorter and PLT concentrator units are connected in serial configuration in continuous processing mode. An embodiment of the microfluidic design layout is illustrated in FIG. 9. Various outlets of both WB sorter and PLT concentrators are connected with fluidic resistance channels to optimize for sorting, concentration and recovery performance.

FIG. 9 shows a microfluidic design of Scheme 1 in continuous operation mode. The network 900 includes a sequence of stages, each having a WB sorter 902 that produces a plurality of plasma rich portions (PRP). The PRP are then connected (not shown) to the PRP inlet 904 of the PLT concentrator 906, which splits into the outer platelet poor plasma (PPP) portions and central platelet poor plasma (PPP) portions as well as the platelet concentrator (PC) outlets.

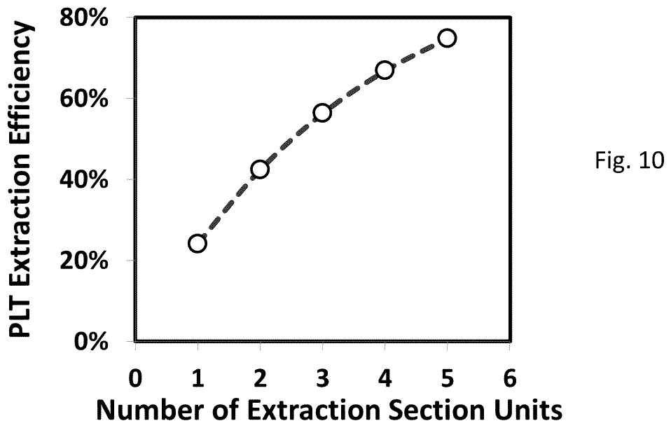

The total PLT extraction efficiency of serially connected WB sorters can be calculated as follow:

$${Extx [ PLT ]}_{Sort}^{Total} = {\sum }_{i = 1}^{{N}_{Sort}} {Extx [ PLT ]}_{Sort ( i )} \qquad [ 5 ]$$

$${Extx [ PLT ]}_{Sort ( i )} = {Extx [ PLT ]}_{Sort}^{Single} \times ( 1 - {\sum }_{i = 1}^{{N}_{Sort} - 1} {Extx [ PLT ]}_{Sort ( i )} ) \qquad [ 6 ]$$

where Nₛₒᵣₜ is the number of WB sorters connected in serial configuration. Extx[PLT]_{Sort}^{Single} is the single pass PLT extraction efficiency of the WB sorter. Extx[PLT]_{Sort(i)} is the PLT extraction efficiency of individual WB sorter unit. According to equations [5], [6], the total PLT extraction efficiency with respect to the number of WB sorters connected in series is plotted in FIG. 10. The equation prediction agrees with experimental data for one (previously characterized at 24%) and two WB sorter units (˜43%). The estimated total extraction for four serially connected WB sorters is 67%.

FIG. 10 shows the PLT extraction efficiency with respect to the number of serial HAR microfluidic sorters. (broken line: theoretical prediction; circles: experimental data)

The WB sorter units 902 and/or the PLT concentrators 906 illustrated in FIG. 9 may be configured as discussed previously to concentrate other types and/or sizes of biological particles in the same or different carrier fluids. This is discussed in more detail with reference to FIGS. 19-37C.

### Microfluidic Design of Parallel Processing in Continuous Operation Mode

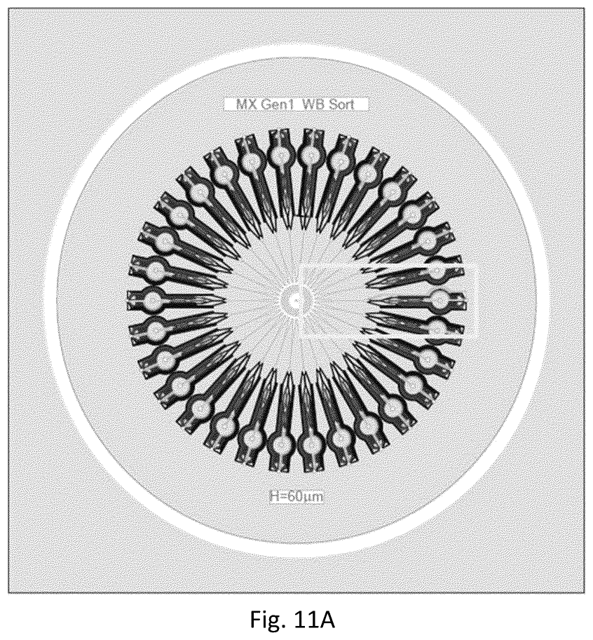

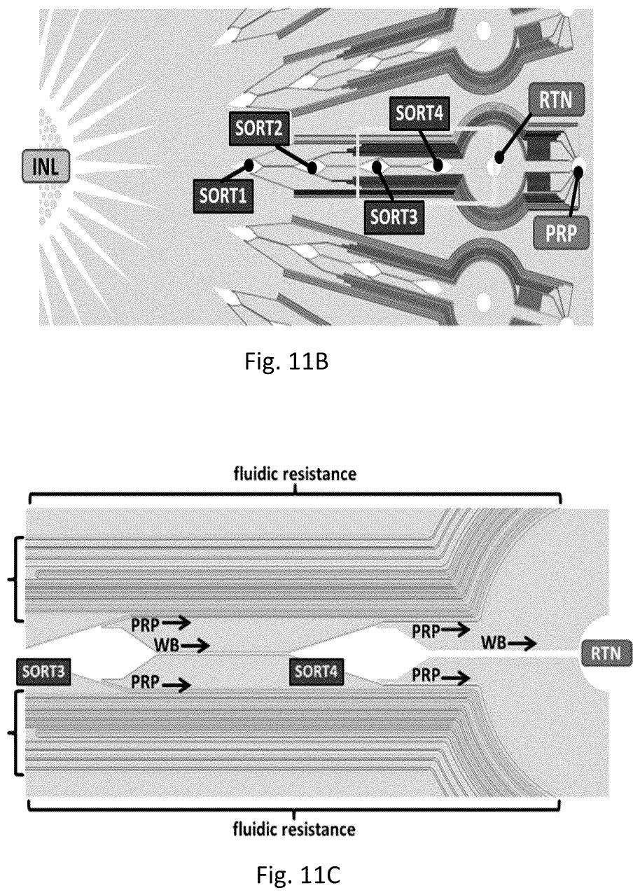

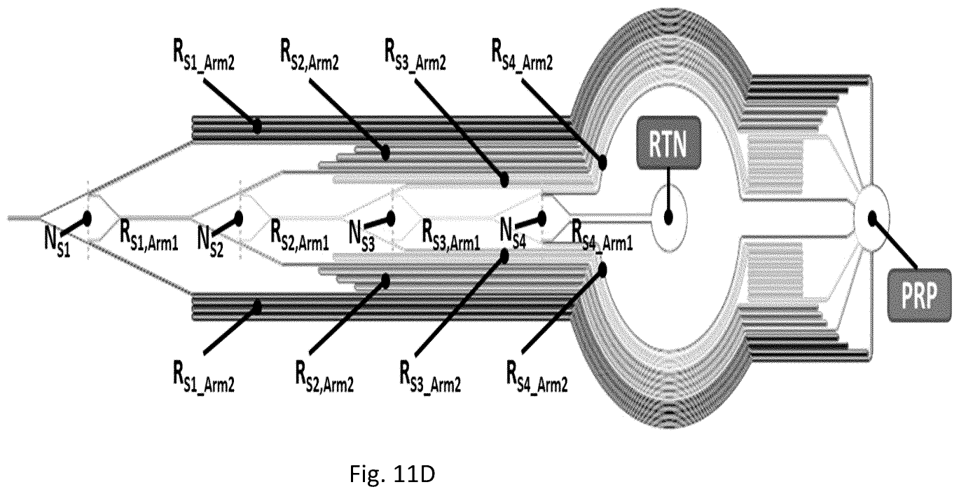

To scale-up throughput rate, multiple microfluidic WB sorter and PLT concentrator channels can connected in parallel (FIGS. 11A-11D). The outlets are grouped together to reduce structure complexity. The outlet channels were also designed to ensure fluidic resistance balance between “Collect” and “Discard” outlets for optimized sample recovery.

The photomask layout of an embodiment is shown in FIG. 11A. The platelet apheresis microfluidic plate contains 30 radially microfluidic branches. Each branch of the microfluidic plate resembles the design of its single-channel counterpart. Each branch containing four serially connected HAR WB sorters is connected to a common inlet at the center (FIG. 11B). The focusing channel of each HAR WB sorter has a cross section of 40 μm×60 μm (W×H). The RBCs experience high inertial force in the focusing channel and migrate to form a core along channel centerline. These abundant and deformable RBCs push smaller and rigid PLTs to marginate (e.g., distribute towards margins) towards the channel side walls. In each WB sorter, RBCs and WBCs are collected by the return (“RTN”) outlet along the center-line in the expansion channel. PLTs are collected by the “PRP” outlet on the side (FIG. 11C). At each bifurcation node of each HAR WB sorter, the desired flow rate split is calculated and designed based on the fluidic resistance balance at each splitting arm. The “PRP/RTN” outlet volume ratio of each HAR sorter is designed to be 1:11 (the volume each “PRP” side outlet, Arm1 is 1:22 of the “RTN” outlet, as is Arm 2) as shown in FIG. 11D. The “PRP/RTN” outlet volume ratio was higher compared to 1:17.5 (each side outlet) in the previous single HAR WB sorter design as to ensure a higher single pass PLT extraction efficiency such that most PLTs can be extracted in the platelet apheresis microfluidic plate.

To reduce the complexity of fluidic circuitry, the “PRP” outlets of all WB sorters can be collected to a common outlet or their own for collection. Blood cells are returned to the donor. The platelet apheresis microfluidic prototypes were fabricated using PDMS soft lithography technique with SU-8 silicon mold. After casting with SU-8 mold, inlet/outlet hole punching and oxygen plasma surface activation treatment, the PDMS piece containing the microfluidic features is bonded with a clean 4″ glass plate. The assembled platelet apheresis microfluidic glass plate device was placed onto a 12 cm petri dish with inlet port encapsulated with another PDMS thick layer to sustain high pressure build-up near the inlet location for higher flow rate operation.

FIG. 11A-11D shows the photomask design for the WB Sort: FIG. 11A shows the overall layout; FIG. 11B shows a zoom-in of each branch; FIG. 11C shows a zoom-in of the fluidic resistance channel; and FIG. 11D outlet fluidic resistance calculation parameters.

Furthermore, WB sorter units and/or the PLT concentrators illustrated in FIG. 11 may be configured as discussed previously to concentrate other types and/or sizes of biological particles in the same or different carrier fluids. This is discussed in more detail with reference to FIGS. 19-37C.

### Quality of PRP Products Subjected to Microfluidic Processing

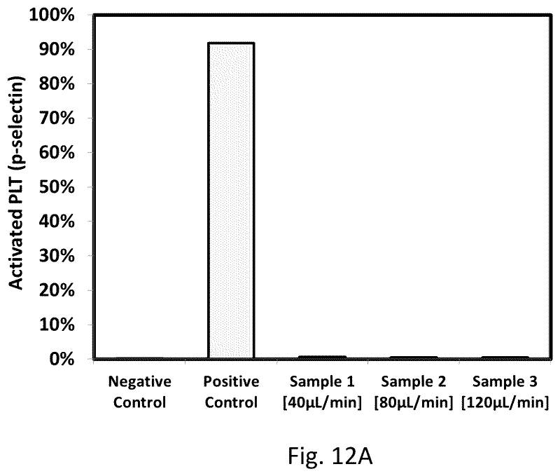

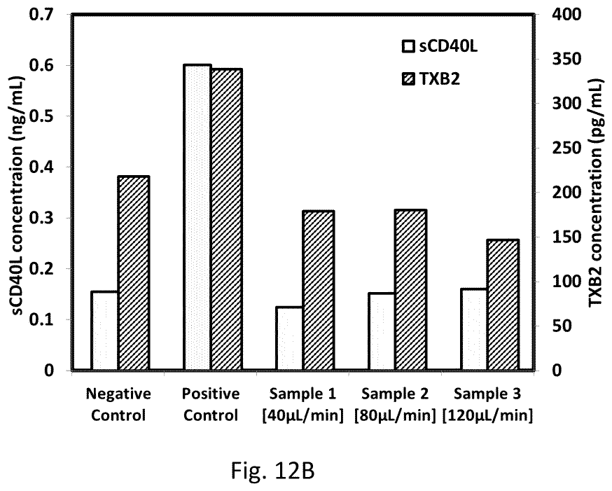

We have shown that PLTs collected from the microfluidic processing maintained good morphology and remained un-activated. We tested the PLT activation status using PLT activation biomarkers both in flow cytometry and ELISA (FIGS. 12A-12B). PRP samples were prepared by centrifugation of WB at 1000×g for 10 minutes and collecting the supernatant. PLTs were collected by processing the PRP samples at flow rates ranging from 40 μL/min to 120 μL/min using the microfluidic sorter devices. Unprocessed PRP aliquot served as the negative control. Positive control was prepared by incubating unprocessed PRP aliquot in 100 nM Phorbol-myristate-acetate (PMA) for 30 minutes at 37° C. to induce platelet activation of CD62 antigen. For flow-cytometry measurements, samples were fixed in 0.5% formaldehyde immediately after sample preparation. Fixed PRP samples were labeled with fluorescently-conjugated anti-CD62P antibody and analyzed using a flow cytometer. Under proper gating and compensation threshold setting, positive control activated by PMA showed greater than 90% PLT activation, while negative control prepared by centrifugation and microfluidic processed samples showed negligible PLT activation, <0.5% (FIG. 12A). Commercial ELISA kits (Human sCD40L, BMS239 from ThermoFisher & Thromboxane B2, ab133022 from Abcam) were used for ELISA assays. Both PLT activation markers (sCD40L & TXB2) showed similar or less measurement level on microfluidic processed samples at all testing flow rates compared with both negative control (centrifugation processed) and positive control (activated by PMA) (FIG. 12B). The low PLT activation level from microfluidic processing can be attributed to the very short time exposure to high shear stress in the small microfluidic focusing channel, which is typically in the order of 10's of milli-second.

### Multi-Layer Microfluidic Cartridges

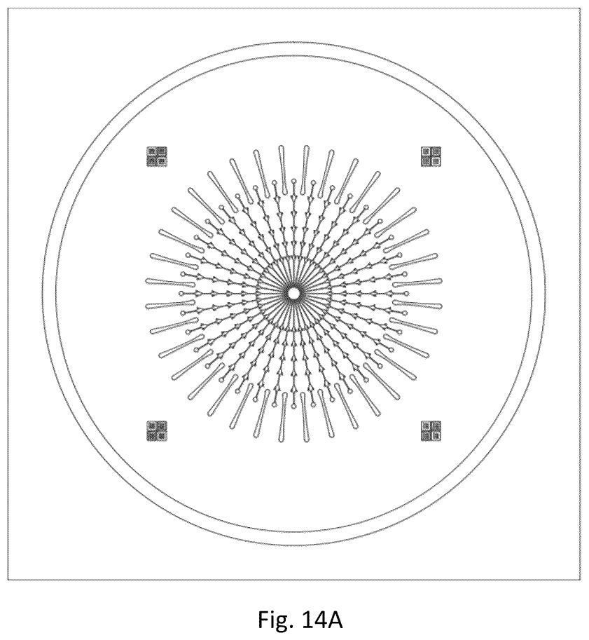

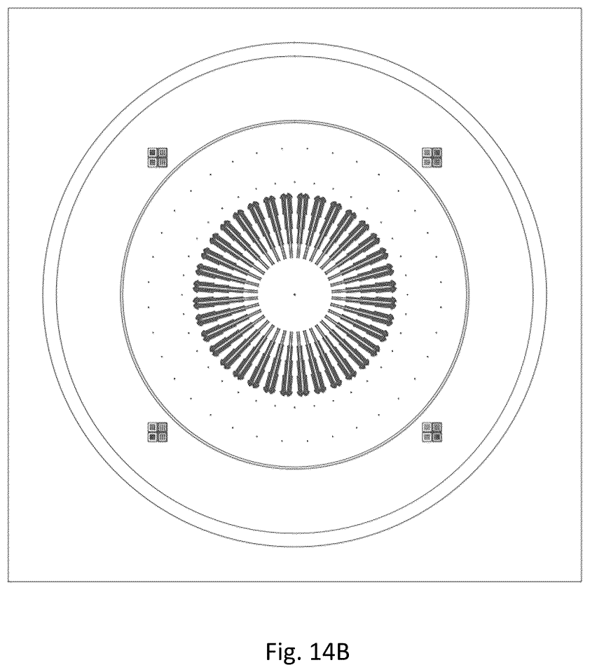

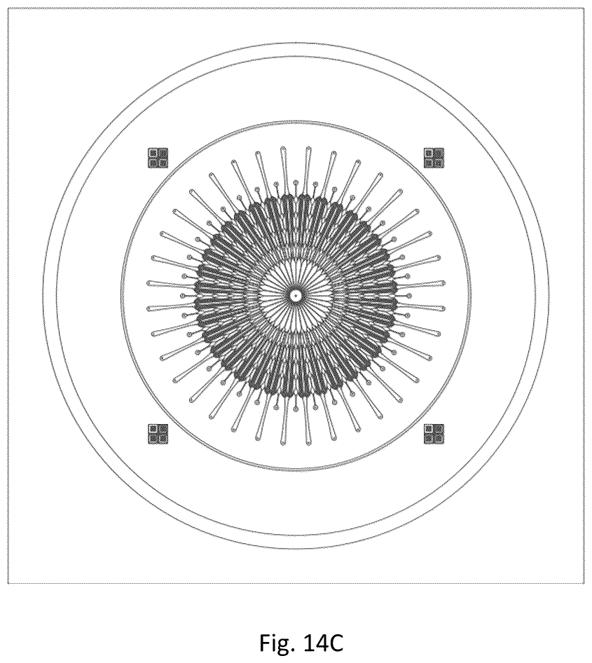

Photomasks for another embodiment of the microfluidic cartridges is a design consisting of two different fluidic heights as shown in FIGS. 14A, 14B, and 14C. The microfluidic platelet extraction cartridge contains 36 radially arranged branches connecting in parallel to a single common inlet at the center. However, other numbers of branches may be used. Furthermore, the microfluidic platelet extraction cartridge may be configured to extract and/or concentrate other types and/or sizes of biological particles in the same or different carrier fluids. This is discussed in more detail with reference to FIGS. 19-37C.

FIGS. 14A-14C shows the photomask designs for two-layer microfluidic cartridge. FIG. 14A shows the mask for the tall central channel region containing multiple sections of the constricting channel followed by expansion regions. FIG. 14B is the mask layer for the shallow side extraction channels that add fluidic resistance to the platelet extraction arms. FIG. 14C is the overlay of the two layers.

Each branch contains five serial expansion sections as shown in FIG. 15A. Under the interaction of hydrodynamic wall lift force, abundant and deformable red blood cells (RBCs) migrated to the channel core in the constricted focusing channel and collected by the main microfluidic channel along the centerline. The plasma and platelets (PLTs) were expelled by the RBC core and marginated near the channel walls. The plasma and PLTs were collected by the side extraction channels at each expansion section, which were in turn connected to common reservoirs on either side of the central channel. The common extraction reservoir was designed with large cross-section, 500×120 μm, W×H to ensure a negligible fluidic resistance. The side extraction channels were designed with a shallower height compared with the main microfluidic channel along the centerline as shown in FIG. 15B. Two-layer design enabled the design to avoid using long meandering high aspect ratio fluidic resistance channels. Instead, the extraction channels were designed to be much shorter and shallower. By design, each microfluidic cartridge was able to sustain a throughput rate of >10 mL/min without leakage. To ensure optimized sorting performance, the desired flow rate split was calculated and designed based on the fluidic resistance balance at each splitting arm of each expansion section. The “EXTX”/“RTN” outlet volume ratio at each expansion section was designed to range from 1:30 to 1:42, resulting in overall volume ratio split of 1:3.

As illustrated in FIG. 15B, the side extraction channels having a shallower height (H2) compared with the height of the main microfluidic channel (H1) may help control the volume of carrier fluid extracted in the side channels. The reduced height of the side channels may create higher fluidic resistance in these pathways compared to the downstream outlet. This increased resistance may effectively limit the volume of carrier fluid directed to the side extraction channels, which may allow for precise control over the separation process or concentration process.

For example, the reduced volume extraction may help focus first biological particles along the centerline of the main channel and ensure that the first biological particles may not be diverted to the shallower side channels. For instance, a carrier fluid may include first biological particles such as bacteria and second biological particles such as phages. The reduced height of the side channels may focus the bacteria in the main channel (e.g., the sorter outlet) and may allow for the phages to be extracted via the side-channels with a reduced amount of bacterial debris and/or contaminants. As a result, a first fraction that is first biological particle poor and/or second biological particle concentrated may be directed to the side channels and a second fraction of the carrier fluid that is first biological particle concentrated and/or second biological particle poor may be directed to the main channel.

The side channel height reduction may be implemented in conjunction with other design parameters, such as the width of the focusing channel and the expansion angle, to improve the separation and/or concentration performance for different types of biological particles. The reduced volume extraction efficiency due to shallower side channels may be compensated for by incorporating multiple sorter or concentrator units in series or by implementing a recirculation strategy to process the carrier fluid multiple times.

One additional advantage of the two-layer design, is that the heights of the layers can be independently varied to achieve different extraction volumes. Through this, system can switch the operational mode from collecting platelet-rich plasma (PRP) from whole blood to collecting platelet-poor plasma (PPP) from PRP allowing the system to produce platelet concentrate (PC) by fabricating devices with different height combinations.

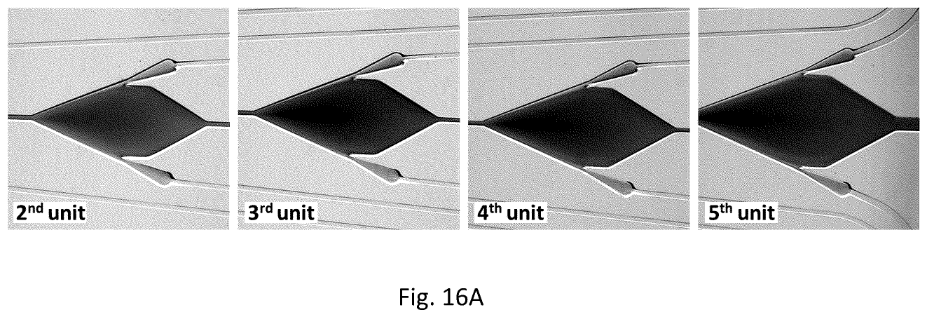

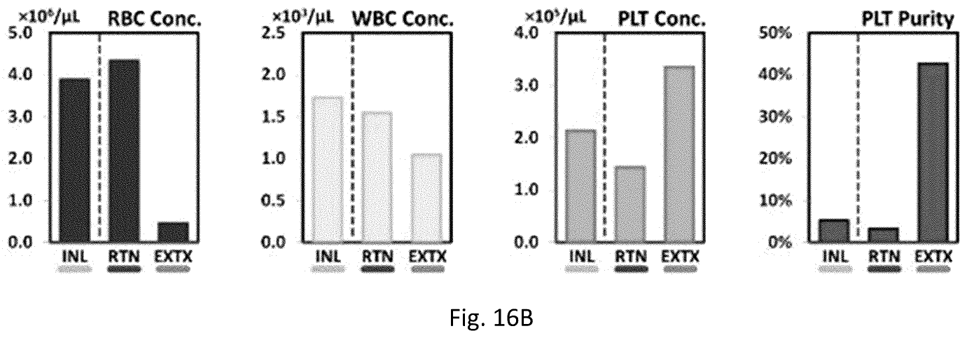

The system used SU-8 silicon masters to cast PDMS substrates for the multi-layer microfluidic cartridges. The cartridge inlet was connected to syringe pump for testing. Prior to blood sorting, the devices were primed by flowing buffer solutions at 5 mL/min in the main fluidic channels and at 1 mL/min in the side extraction channels (back-flow). Freshly collected blood from a donor by venipuncture infused with 10% ACD-A was used as the inlet sample. After priming the device with buffer, we collected WB samples in 50 mL syringe and used a syringe pump to infuse to each device at 20 mL/min for 2.5 min. No leakage was encountered for each device. The RBC flow trajectories in real-time were imaged under an optical microscope at different locations of the device at one branch as shown in FIG. 16A. Most RBCs were collected along the main microfluidic channel for all expansion sections. A small portion of RBCs were collected to the side extraction channels. The processed samples were cell counted under an epifluorescence microscope. The RBC portion was counted in bright field imaging with 100× dilution in 10% ACD 1×PBS buffer. The PLT and WBC portion were counted with 10× dilution in 10% ACD 1×PBS buffer with Calcein AM staining. Cells with >4 μm diameter were counted as WBCs, while cells with diameter <4 μm were counted as PLTs. Sorting performance is shown in FIG. 16B. Multi-layer devices resulted in RBC concentration discard of >97% and PLT purity ˜40% in the “EXTX” samples in single pass operation. The PLT purity was about 40% with the PLT extraction of ˜30%.

### Collection Manifold for Platelet Extraction Plates

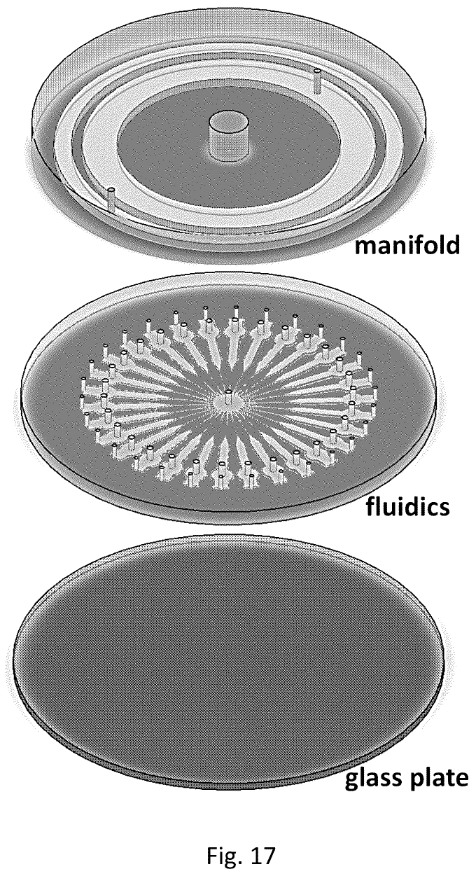

The PDMS manifold can be configured as in FIG. 17, which connects all common outlets from the fluidics layer of the microfluidic platelet extraction plates to avoid extensive use of connection tubing. The PDMS manifold consisted of two annular rings. The inner ring connected all “RTN” outlets from the fluidics layer to one common outlet. The outlet ring connected all EXTX outlets from the fluidics layer to another common outlet. These inlets and common outlets were connected to syringe, reservoirs, or other fluidic components through a single Tygon™ tubing (0.050″ ID, 0.090″ OD). The center inlet was inserted with a Luer fitting, which can be detached from the microfluidic platelet extraction plate. The PDMS manifold can be repeatedly cast with micro-machining of a thin polycarbonate mold. After fabricating the microfluidic platelet extraction plate, the PDMS manifold was aligned and capped on top of the fluidic layer after cleaning and surface activation with oxygen plasma treatment, followed with bonding on a hot plate. A glass plate was bonded using a hot-plate on the bottom of the microfluidic platelet extraction plate after cleaning and surface activation with oxygen plasma treatment. A 1/16″ barbed straight connector was inserted into each outlet on the manifold and a Luer connector was inserted into inlet port. The entire microfluidic assembly was encapsulated with another PDMS stack on a petri dish to sustain high pressure build-up near the inlet location for higher flow rate operation.

The microfluidic assembly described with reference to FIG. 17 may be modified with different microfluidic plates configured to separate and/or concentrate other types and/or sizes of particles as is described in more detail with reference to FIGS. 19-37C.

### Automated Dual-Pump Operation of the Platelet Apheresis Cartridges

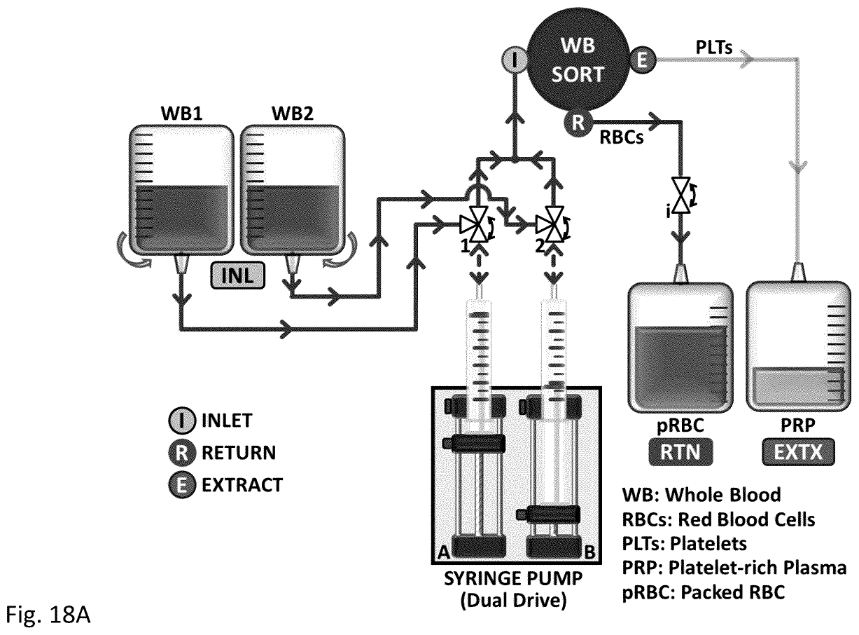

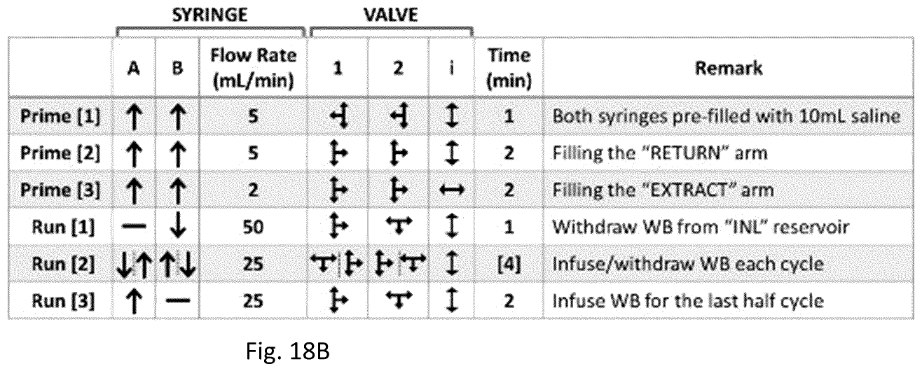

In some embodiments, the platelet apheresis cartridges were operated using dual syringe pump as in FIG. 18A. At any given time, one syringe is operated in the infuse mode to provide blood to the microfluidic device, and the other syringe is operated in the withdraw mode to pull blood from the whole blood input reservoir. When the infusing syringe is emptied out, the valves connected to the syringes are actuated such that the infusing syringe switches to the withdraw mode and the withdrawing syringe switches to the infuse mode. The process is repeated throughout the apheresis operation. The valve actuation time is of the order of 1 sec causing minimal disruption in the flow to the device. 3D-printed valve actuation fixtures controlled via a bread-board controller is utilized for automated operation of the valves. The bread-board controller is based on the Arduino Uno microcontroller and includes a MotorShield capable of driving multiple rotational actuators. Custom-designed 3D printed parts are fabricated that enclose the three-way valves as well as provide connectivity to the rotational actuator. The automated setup can be utilized to process large blood volumes (˜1-1.5 L).

The valve actuation steps for the different processing steps: priming, and running, are as shown in FIG. 18B. The priming steps sequences consist of: filling the syringes with 10 mL of saline, priming the RETURN arm of the fluidic cartridge, and priming the EXTRACT arm of the fluidic cartridge. The running steps consist of: withdrawing WB in one syringe of the pump from the INL reservoir, infusing WB from the filled syringe while simultaneously withdrawing blood into the empty syringe, repeating the preceding step as needed, and finally infusing WB out from the filled syringe.

### Particle Separation or Concentration

FIG. 19 is a top view or longitudinally oriented cross-sectional view of a microfluidic network 1900 that may be used for phage particle separation or concentration. The microfluidic network 1900 may be included in a carrier fluid sorter (e.g., the WB sorter), which may be configured for the separation and/or concentration of particles other than platelets. The microfluidic network 1900 may be included in a device with similar components as the portable platelet apheresis device 100, but may be configured for the separation and/or concentration of particles other than platelets such as phage particles.

The microfluidic network 1900 may include an inlet that is configured to receive carrier fluid from a carrier fluid source. In these and other embodiments, the carrier fluid may be whole blood, Luria-Bertani (LB) media, plasma, or other suitable carrier fluids which may include particles and may allow for the separation and/or concentration of particles. In some embodiments, the carrier fluid may include biological particles such as viruses (e.g., phages), bacteria, endotoxins, liposomes, extracellular vesicles (EV), exosomes, nucleic acids, proteins, and/or polypeptides. In some embodiments, the carrier fluid may include first biological particles and second biological particles that are different in size and/or type of the first biological particles. For example, the second biological particles may be smaller than the first biological particles.

The inlet may include a narrowing tapered region 1902 having a narrowed outlet 1904 that may be coupled to and/or include of a constricted region 1906. The constricted region 1906 extends to a constricted outlet 1908 that is fluidly coupled with an expansion region 1910, which is expanded relative to the constricted outlet 1908. In some embodiments, the expansion region 1910 may include an expanded region (e.g., the expanded region 212). In some embodiments, the expansion region 1910 may include at least one side channel formed into the side of the expansion region 1910 (e.g., similar to the side channel outlets 214 shown in FIG. 2A). The microfluidic network 1900 may include at least one sorter outlet (e.g., similar to the downstream outlet 216 shown in FIG. 2A) that is downstream or medial from the at least one sorter side channel. In some embodiments, the expansion region may be divided into a plurality of downstream outlets that are downstream and/or medial to the side channel outlets.

The microfluidic network 1900 may include a straight constricted microfluidic channel followed by an expansion microfluidic channel having at least two exits. The microfluidic channels may have various cross-sectional dimensions at the constricted region 1906 and the expansion region 1910 as shown in FIG. 19. For example, the constricted region 1906 may have a first cross-sectional dimension and the expansion region 1910 may have a second cross-sectional dimension that is larger than the first cross-sectional dimension. For instance, the expansion region 1910 may be at least 1.2 times larger than the constricted region 1906, at least about 1.5 times larger than the constricted region 1906, at least about 2 times larger than the constricted region 1906, at least about 3 times larger than the constricted region 1906, or at least about 4 times larger than the constricted region 1906. In some embodiments, the constricted region 1906 may include a cross-sectional dimension of at least about 20 microns, or from about 20 microns to about 100 microns, but could be larger if needed or desired (e.g., 200, 300, or up to 500 microns).

In some embodiments, the constricted region 1906 may have a width between about 2 micrometers and about 30 micrometers. In these and other embodiments, the constricted region 206 may have a length between about 10 micrometers and about 2 centimeters. In these and other embodiments, the constricted region 1906 may have a height between about 10 micrometers and about 120 micrometers. In some embodiments, and as described in more detail with reference to FIG. 15B, the height of the side channel outlets may be less than the height of the downstream outlet such that the fluidic resistance in the side channel outlets may direct a higher volume of the carrier fluid to the downstream outlet.

The microfluidic network 1900 may be configured to direct a first fraction of the carrier fluid to the at least one side channel and a second fraction of the carrier fluid to the downstream outlet. As mentioned previously, the carrier fluid may include first and/or second biological particles that may be smaller than the first biological particles. In some embodiments, the first fraction of the carrier fluid may be first biological particle poor and the second fraction of the carrier fluid may be first biological particle concentrated. For example, the first fraction may have a lower concentration of first biological particles than the second fraction. Thus, the microfluidic network 1900 may concentrate the first biological particles in the carrier fluid such that the first biological particles may be concentrated in the carrier fluid received by the at least one downstream outlet. In some embodiments, the first fraction of the carrier fluid may be second biological particle concentrated and the second faction may be second biological particle poor. In some embodiments, the first fraction may be first biological particle poor, but second biological particle concentrated, and the second fraction may be first biological particle concentrated, but second biological particle poor. Thus, the microfluidic network 1900 may separate first biological particles and the second biological particles in the carrier fluid such that a first biological particle concentrated fraction may be directed to the at least one downstream outlets and the second biological particle concentrated fraction may be directed to the side outlets.

Because particles may be differently sized, the microfluidic network 1900 may be configured to leverage principles of inertial microfluidics to modify wall lift forces to separate and/or concentrate particles based on these size differences.

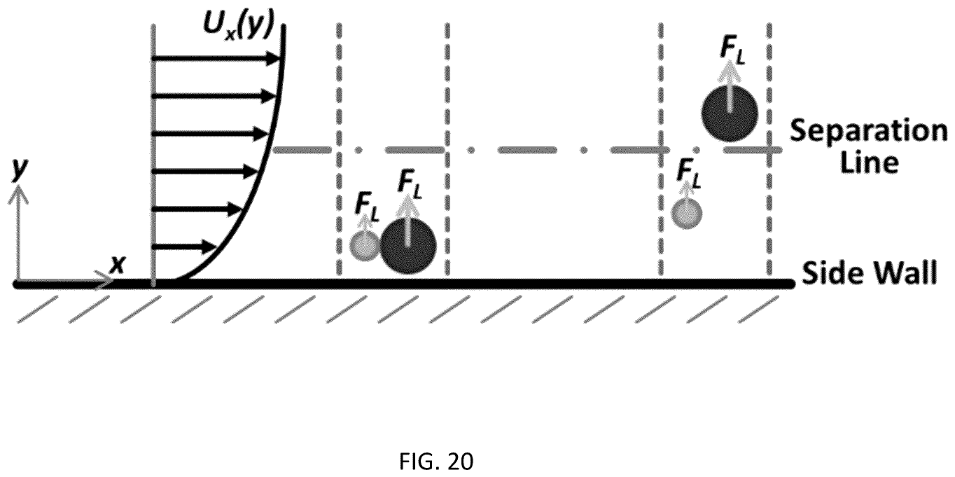

As an example, FIG. 20 is a schematic showing the variation in lift relative to the side wall of a microfluidic network for different particle sizes as the particles progress through the microfluidic network. As illustrated in FIG. 20, different sized particles will experience varying magnitudes of wall lift forces when flowing through the microfluidic network of the microfluidic separation system. Larger sized particles may experience higher wall lift forces while smaller sized particles may experience lesser wall lift forces. As a result, larger particles may be separated from the smaller particles in the carrier fluid and may move to the microfluidic channel centerline at a greater rate than smaller particles. Thus, the smaller particles illustrated in FIG. 20 may be second biological particles of a smaller size that may be concentrated in the first fraction of the carrier fluid and the larger particles may be first biological particles of a larger size that may be concentrated in the second fraction of the carrier fluid.

The wall lift force may cause particles to move away from the channel walls toward equilibrium positions in the carrier fluid. For larger particles, this equilibrium position may be closer to the channel centerline due to the stronger wall lift forces. Smaller particles, experiencing weaker wall lift forces, may migrate more slowly and may be distributed more widely across the channel width.

Based on inertial microfluidics, the microfluidic network may be configured differently to achieve different wall lift forces depending on the types and/or sizes of particles to be separated and/or concentrated. Thus, the microfluidic network may control the positions of the first biological particles and/or second biological particles in the carrier fluid such that biological particles may be separated and/or concentrated.

For example, the constricted region may be narrower (e.g., reduced width) and/or shorter (e.g., reduced height), which may generate stronger wall lift forces. As a result, the particles may experience a greater degree of separation in the carrier fluid. Similarly, a longer constricted region may provide more time for differently sized particles to reach their equilibrium positions, which may also enhance separation and/or concentration.

In the expansion region downstream from the constricted region, the differential focusing of particles based on size may result in spatial separation. Larger particles may be concentrated near the channel centerline, while smaller particles may be distributed more broadly. This spatial separation may allow for the collection of size-fractionated particles through appropriately positioned side outlet channels. The angle and/or size of the expansion region may also be adjusted to aid in the separation and/or concentration of particles. For example, the angle of the expansion region may affect how well the spread of the particles are maintained as the particles exit the focusing channel. A gradual expansion may help preserve the spatial separation achieved in the focusing channel, while a sudden expansion may cause mixing of the particles.

Additionally, the flow rate may be adjusted according to desired inertial forces and diffusion. Higher flow rates may increase inertial effects due to the increased pressure but may also reduce the residence time in the focusing channel. Lower flow rates may provide more time for focusing but may reduce the throughput of the microfluidic network.

By controlling at least some of the above described parameters in the design and operation of the microfluidic network in the carrier fluid sorter, the inertial microfluidic system may effectively separate particles with different morphological characteristics, such as size without requiring external forces or membrane filtration.

Returning to FIG. 19, the microfluidic network 1900 may be used for phage particle separation or concentration. The microfluidic network 1900 may include an inlet that receives carrier fluid including the phage particles from a carrier fluid source. The carrier fluid may flow through a narrowing tapered region 1902 having a narrowed outlet 1904 that may be fluidly coupled to a constricted region 1906. The constricted region 1906 may extend to a constricted outlet 1908 that may be fluidly coupled with an expansion region 1910. As illustrated in FIG. 19, the carrier fluid may include a first group of phage particles and a second group of phage particles, the first group of phage particles may be larger than the second group of phage particles.

As the carrier fluid including phage particles flows through the constricted region 1906, the phage particles may experience wall lift forces that vary based on the size of the particles. Larger phage particles may experience stronger wall lift forces compared to smaller phage particles. The differential wall lift forces may cause the larger phage particles to migrate more rapidly toward the channel centerline, while smaller phage particles may remain distributed more widely across the channel width.

When the carrier fluid exits the constricted region 1906 and enters the expansion region 1910, the spatial separation of differently sized phage particles may be maintained. As illustrated in FIG. 19, a first fraction (1) of the carrier fluid may be substantially free of phage particles, a second fraction (2) of the carrier fluid may be concentrated with smaller phage particles, and third fraction (3) of the carrier fluid may be concentrated with larger phage particles, but may also include some of the smaller phage particles.

The microfluidic network 1900 may be configured differently depending on whether phage particles are to be separated or concentrated. As illustrated in FIG. 19, in an example concentration operation of the carrier fluid sorter, the microfluidic network may have three outlets (e.g. two side channels and one downstream outlet as illustrated with respect to at least FIG. 2A, 24A, 32A) positioned to concentrate the phage particles in the carrier fluid (as illustrated by the brackets under the “Virus Conc.” heading), the phage-free fraction (1) may be directed to the side channels (shown by the smaller brackets under the “Virus Conc.” heading), and the second and third fractions (2) and (3) with the smaller and larger phage particles may be directed to the downstream outlet (shown by the larger bracket under the “Virus Conc.” heading). In some embodiments, the carrier fluid may further include endotoxins, which may be included in the first fraction of the carrier fluid and separated from the phage particles (both small and large). In these and other embodiments, the phage particles may be considered first biological particles and the endotoxins may be considered second biological particles. Thus, the phage-free or phage-poor fraction may be removed from the carrier fluid via the side channels, which may reduce the overall volume of the carrier fluid, but the phage-concentrated fraction may be directed to the downstream outlet.

In some embodiments, the microfluidic network 1900 may be configured and/or operated to direct the smaller and the larger phage particles to the downstream outlet. For example, the constricted region 1906 and/or the expansion region 1910 may be configured to modify the wall-lift force on both the smaller and larger phage particles such that the phage particles are directed to the downstream outlet. For example, the constricted region may have a width of about 10 micrometers, a length of about 2 centimeters, and/or a height of about 50 micrometers. In some embodiments, the width may be between about 2 micrometers and about 10 micrometers, the length may be between about 0.2 millimeters and about 2 centimeters, and/or the height may be between about 10 micrometers and about 250 micrometers.

Furthermore, the flow rate may be modified to direct the phage particles to the downstream outlet and/or fluidic resistance at the side channels may be modified (e.g., via a smaller height compared to the downstream outlet or resistance created via other techniques) to direct the phage particles to the downstream outlet. For example, the flow rate may be about 40 microliters per minute. In some embodiments, the flow rate may be between about 2 microliters per minute and about 500 microliters per minute.

In a 2 micrometer wide constricted region 1906 with a single constricted region and a single expansion region 1910, a 15% increase in phage concentration was seen. In some embodiments, the carrier fluid may be recirculated through the microfluidic network 1900 and/or circulated through additional microfluidic networks 1900 to obtain a purer solution of phage particles. It is estimated that continuous processing (e.g., re-circulation of the concentrated phage fraction) may improve concentration of phage particles by greater than 10× after 6 or 7 recirculation passes.

As illustrated in FIG. 19, in an example separation operation of the carrier fluid sorter, the microfluidic network may have three outlets (e.g. two side channels and one downstream outlet as illustrated with respect to at least FIG. 2A, 24A, 32A) positioned to separate the larger particles and the smaller particles in the carrier fluid (as illustrated by the brackets under the “Virus Sort.” heading), the phage-free fraction (1) and the second fraction (2) that is smaller phage concentrated may be directed to the side channels (shown by the smaller brackets under the “Virus Sort.” heading), and third fraction (3) that is larger phage concentrated may be directed to the downstream outlet (shown by the larger bracket under the “Virus Sort.” heading). In these and other embodiments, the larger phage particles may be considered first biological particles and the smaller phage particles may be considered second biological particles. Thus, a smaller phage particle concentrated and a larger phage particle poor fraction of the carrier fluid may be removed via the side channels, and a smaller phage particle poor and larger phage particle concentrated fraction of the carrier fluid may be directed to the downstream outlet.

In some embodiments, the microfluidic network 1900 may be configured and/or operated to direct the larger phage particles to the downstream outlet. For example, the constricted region 1906 and/or the expansion region 1910 may be configured such that the wall-lift force allows the smaller particles to be removed via the side channels and the larger phage particles to be directed to the downstream outlet. For example, the constricted region 1906 may have a width of about 2 micrometers, a length of about 10 micrometers, and/or a height of about 14 micrometers. In some embodiments, the width may be between about 2 micrometers and about 10 micrometers, the length may be between about 0.2 millimeters and about 2 centimeters, and/or the height may be between about 10 micrometers and about 250 micrometers.

Furthermore, the flow rate may be modified to direct the larger phage particles to the downstream outlet and/or fluidic resistance at the side channels may be increased (e.g., via a smaller height compared to the downstream outlet or resistance created via other techniques) to direct the phage particles to the downstream outlet. For example, the flow rate may be about 5 microliters per minute. In some embodiments, the flow rate may be between about 2 microliters per minute and about 500 microliters per minute.

In some embodiments, the carrier fluid may be recirculated through the microfluidic network 1900 and/or circulated through additional microfluidic networks 1900 to obtain a purer solution of large phage particles.

Thus, the microfluidic network 1900 may be configured to separate larger phage particles from smaller phage particles or separate phage particles from other particles such that the phage particles may be concentrated in the carrier fluid. Furthermore, the microfluidic network 1900 may also be modified and/or configured to separate and/or concentrate other particles such as liposomes, exosomes, EVs, bacteria, white blood cells, red blood cells, platelets, nucleic acids, proteins, and/or polypeptides, among others.

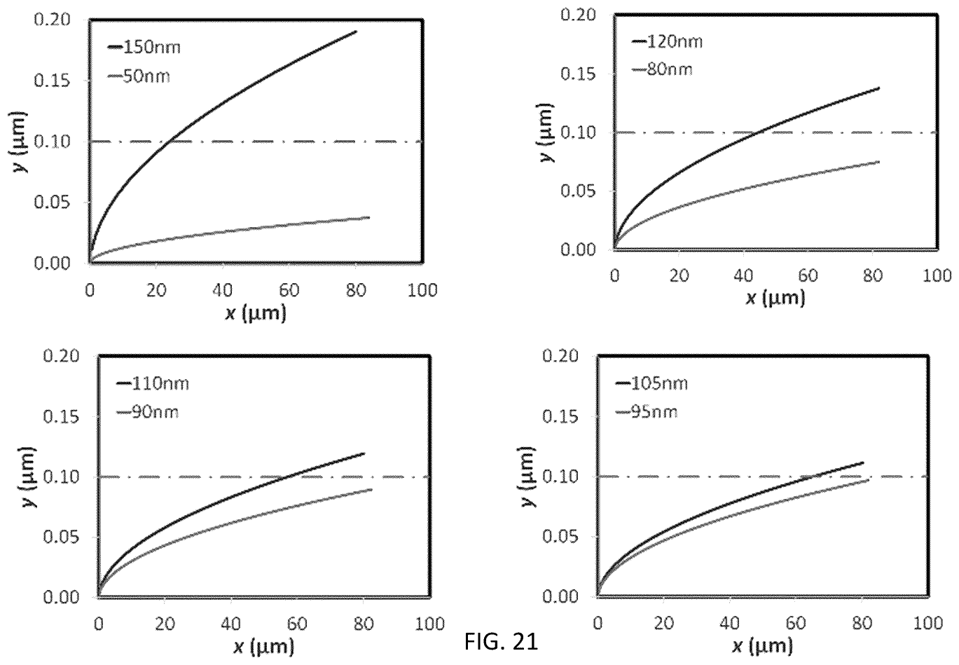

FIG. 21 illustrates predicted flow trajectories of different size pairs of phage particles near the side wall. A two-dimensional model was used to predict the separation resolution of two phage particles of different size in a focusing channel (e.g., the sorter constricted region). The particle lateral migration velocity (UL) may be determined as follows:

$${U}_{L} = \frac{{F}_{L}}{3 πμ {a}_{p}} = \frac{2 ρ {{U}_{f}}^{2} {{a}_{p}}^{3}}{3 πμ {{L}_{c}}^{2}}$$

where F_{L} is the wall lift force, μ and ρ are the fluid dynamic viscosity and density respectively, ap is the particle diameter, U_{f} is the fluid velocity and L_{c} is the focusing channel characteristic length (which was equivalent to the width in the case of high aspect ratio channel). The fluid velocity profile for pressure-driven flow may be determined as follows:

$${U}_{x} ( y ) = 4 {U}_{f} \frac{y}{{L}_{c}} ( 1 - \frac{y}{{L}_{c}} )$$

The graphs depicted in FIG. 21 illustrate flow trajectories of particle pairs where one particle is larger than another particle in a constricted region having a width of 2 micrometers and a flow rate of 120 microliters per minute. The upper-left graph illustrates particle separation between a first particle having a 150 nm diameter and a second particle having a 50 nm diameter, the upper-right graph illustrates particle separation between a second particle having a 120 nm diameter and a second particle having an 80 nm diameter, the bottom-left graph illustrates particle separation between a first particle having a 110 nm diameter and a second particle having a 90 nm diameter, and the bottom-right graph illustrates particle separation between a first particle having a 105 nm diameter and a second particle having a 95 nm diameter. The dashed line illustrates a separation line at 0.10 micrometer in lift from the sidewall at which the different particle sizes may be separated by a microfluidic network. Thus, the model may be utilized to predict, for example, the desired length of the focusing channel in order for the larger particle to be separated from the smaller particle. For instance, each of the graphs show that for this particular width and flow rate in the focusing channel, a length of 80 micrometers may allow each of the particle size pairs to be separated (e.g., the larger particle being above the separation line and the smaller particle being below the separation line).

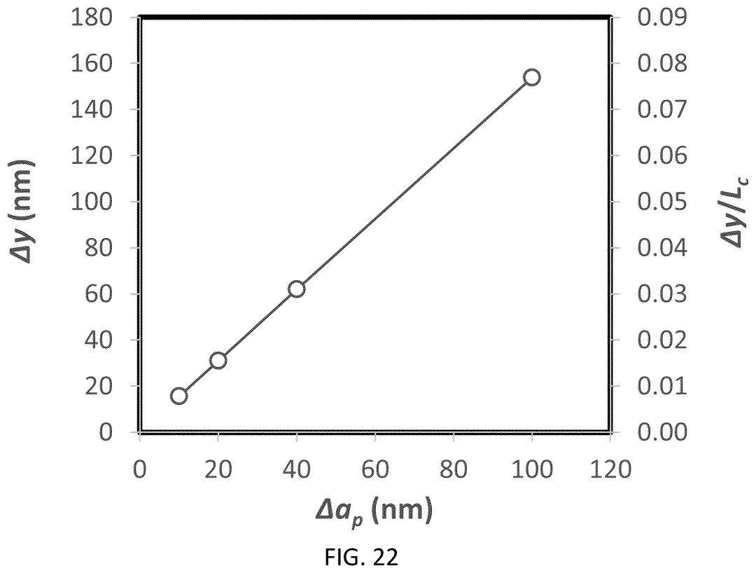

FIG. 22 illustrates the predicted separation at the outlet of the focusing channel for the different phage particle size pairs illustrated in FIG. 21 with a length of 80 micrometers. The x-axis illustrates the difference in particle size between the particle size pairs, the first Y-axis (left) illustrates the separation between the two particles in nanometers, and the second Y-axis right) illustrates the change in separation per unit change of focusing channel length. The separation at the outlet of the focusing channel (e.g., the constricted outlet 1908) may be determined using the equation fitting the data illustrated in FIG. 22:

$$Δ y = 1.54 Δ {a}_{p} + 0.35$$

At the microfluidic focusing channel outlet (e.g., the constricted outlet 1908), most larger particles (e.g., large phage particles) will be inertially focused by the wall lift force (e.g., the third fraction (3) in FIG. 19), leaving a small region composed of pure population of smaller particles (e.g., smaller phage particles) near the side walls (e.g., the second fraction (2) in FIG. 19). The small extraction efficiency can be compensated by re-circulation of the third fraction (3) back to the inlet for continuous particle sorting and/or additional circulation of third fraction (3) with additional carrier fluid sorters.

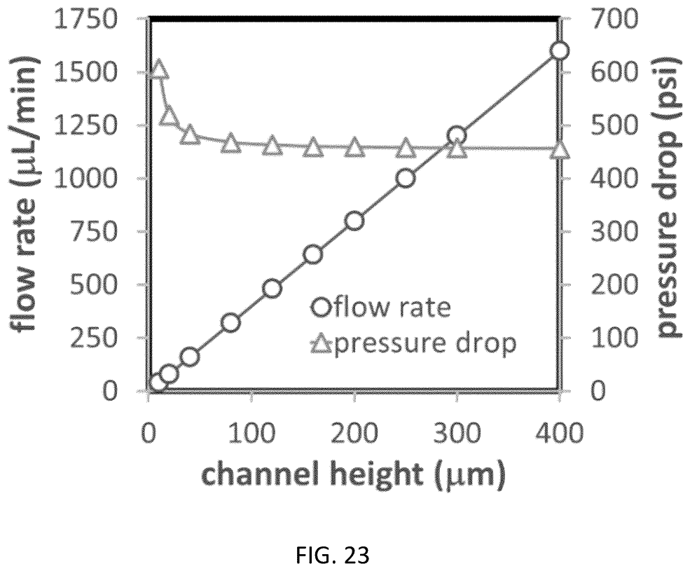

FIG. 23 illustrates expected flow rates of increasing channel height in a microfluidic separation system configured for phage separation. In the graph depicted in FIG. 23, a focusing channel having a 4 micrometer width, a 250 micrometer length was used, and the height was varied to evaluate the effect of channel height on flow rate through the microfluidic network. The focusing channel dimensions may be specifically used to focus 250 nanometer particles. FIG. 23 illustrates that the pressure drop decreased slightly with increasing channel height and approached a constant value for larger channel heights. FIG. 23 also illustrates that increasing focusing channel height may improve throughput rate.

As the channel height is increased, the flow rate may need to be increased to match the same fluid velocity to generate the same lift force to create separation of particles including phage particles. The pressure drop may decrease slightly with increasing channel height and may approach a constant value for larger channel heights. This relationship suggests that increasing the channel height may improve throughput without requiring proportionally higher pressure to drive the carrier fluid through the microfluidic network.

Thus, by adjusting the channel height, the system may be tuned to achieve higher throughput while maintaining manageable pressure, which may allow processing of larger carrier fluid volumes.

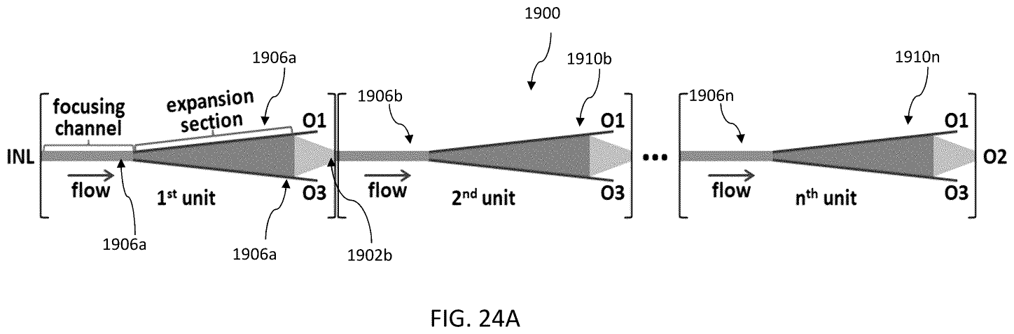

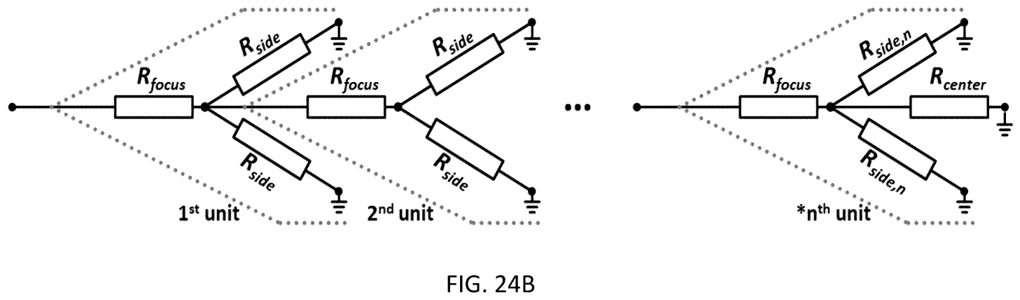

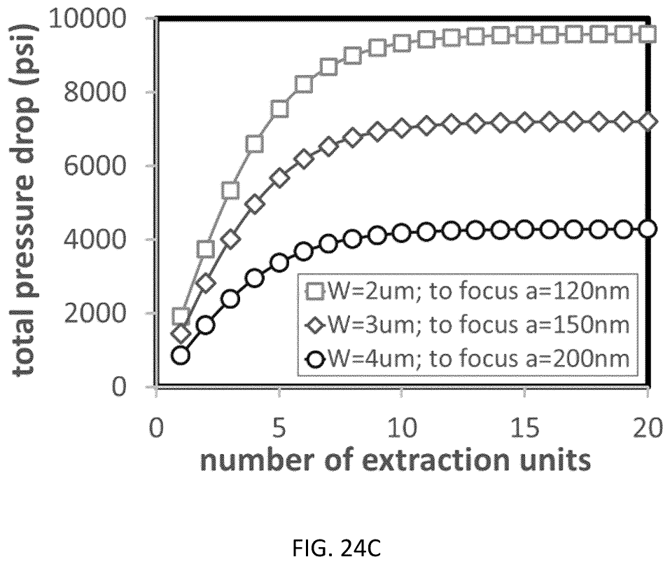

FIG. 24A illustrates an example microfluidic separation system with multiple carrier fluid sorters units connected in series and FIG. 24B illustrates an equivalent electrical circuit for the microfluidic separation system of FIG. 24A. As illustrated in FIG. 24A, and as previously described with respect to at least FIG. 8 and FIG. 11B, the carrier fluid sorter may be configured for serial connection of carrier fluid sorters each having the microfluidic network 1900. For example, the microfluidic network 1900 may include a first unit with first constricted region 1906a and a first expansion region 1910a, a second unit with a second constricted region 1906b and a second expansion region 1910b, and/or additional units (nth units) having additional constricted regions 1906n and additional expansion regions 1910 with each unit having side channels O1 and O2 and a downstream outlet O3.

The serial connection of multiple units may improve the removal rate of second biological particles and/or the concentration of first biological particles. For example, the endotoxin and small contaminant removal rate may be improved (e.g., a higher number of endotoxin particles may be directed to the side channels O1 and O2) and the number of phage particles directed to the downstream outlet O3 may be increased.

FIG. 24C illustrates expected total pressure drop (psi) for different carrier fluid sorter microfluidic network configurations having a different number of units (e.g., extraction units). The square markers illustrate the pressure drop of a carrier fluid sorter having various numbers of extraction units and focusing channel dimensions of 2 micrometers in width, 150 micrometers in length, 200 micrometers in height, and a flow rate of 0.7 milliliters/minute to focus 120 nanometer phage particles (e.g., to direct the first fraction to the sorter outlet O2). The diamond markers illustrate the pressure drop of a carrier fluid sorter having various numbers of extraction units and focusing channel dimension of 3 micrometers in width, 400 micrometers in length, 300 micrometers in height, and a flow rate of 1 milliliter/min to focus 150 nanometer phage particles (e.g., to direct the first fraction to the sorter outlet O2). The circular markers illustrate the pressure drop of a carrier fluid sorter having various numbers of extraction units and focusing channel dimensions of 4 micrometer in width, 500 micrometers in length, 400 micrometers in height, and a flow rate of 1.5 milliliters/minute to focus 200 nanometer phage particles (e.g., to direct the first fraction to the sorter outlet O2).

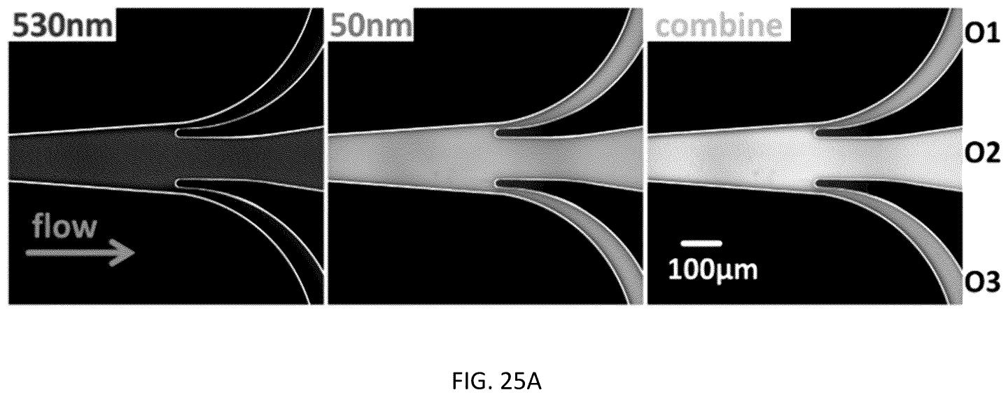

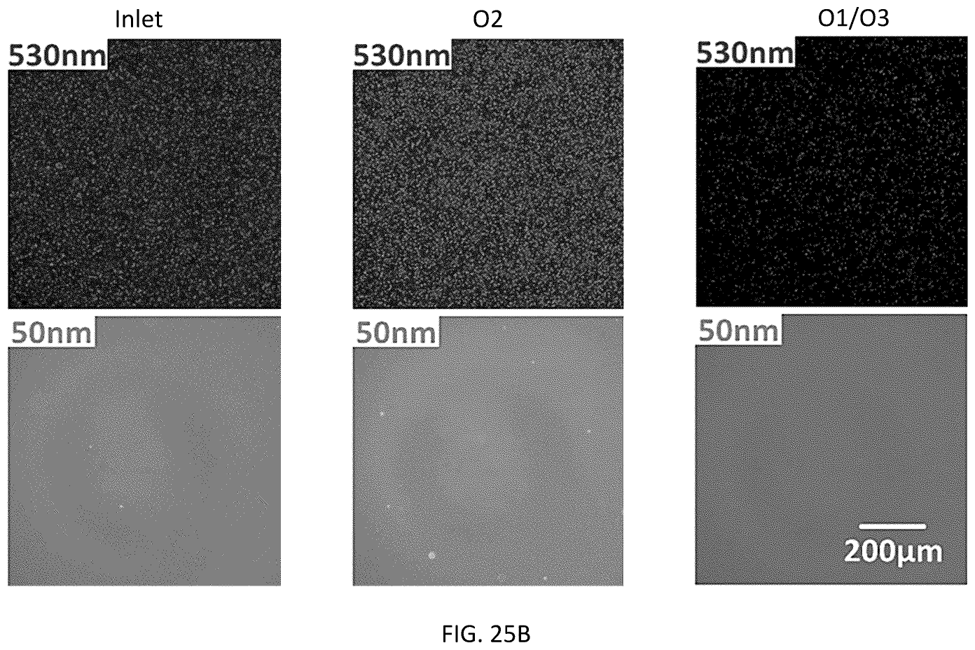

FIGS. 25A, 26A, 27A, 28A, and 29A show the separation of a first group of particles having a larger size from a second group of particles having a smaller size in an expansion region of a microfluidic network having focusing channels of varying dimensions and varying flowrates. FIGS. 25B, 26B, 27B, 28B, and 29B each show fluorescent images of samples taken from the inlet, the side channels (O1, O3), and the sorter outlet (O2) respectively for FIGS. 25A, 26A, 27A, 28A, and 29A.

Two of the following fluorescent microspheres were used in FIGS. 25-27 to demonstrate the separation of differently sized particles: (1) SPHERO™ Fluorescent Nile Red Microspheres, 0.53 μm, 1% w/v (Catalog #: FP-0556-2) from Spherotech® Inc., (2) Fluoresbrite® YG (green) Microspheres, 0.05 μm, 2.5% aqueous suspension (Catalog #: 17149-10) from Polysciences®, Inc, (3) Fluoresbrite® YG Carboxylate Microspheres 0.30 μm, 2.5% aqueous suspension (Catalog #: 24051-10) from Polysciences®, Inc. or (4) Fluoresbrite® YO Carboxylate Microspheres 0.05 μm, 2.5% aqueous suspension (Catalog #: 19775-10) from Polysciences®, Inc.

FIG. 25A shows the separation of a first group of particles having a size of 530 nm and a second group of particles having a size of 50 nm in an expansion region of a microfluidic separation system having a focusing channel (e.g., a sorter constricted region) of 7 micrometer width, 120 micrometer length, and 50 micrometer height at a carrier fluid flow rate of 60 μL/min. As illustrated, the sorter has side channels outlets O1 and O3 and a downstream outlet (e.g., a sorter outlet) O2.

Specifically, the first group of particles were SPHERO™ Fluorescent Nile Red Microspheres, 0.53 μm (530 nm) and the second group of particles were Fluoresbrite® YG Microspheres, 0.05 μm (50 mm). Thus, the microspheres used may mimic separation of first biological particles having a 530 nanometer diameter and second biological particles having a 50 nanometer diameter.

20 μL Nile Red and 10 μL YG fluorescent microsphere solutions were mixed in 100 μL 1×PBS and sonicated for an hour before adding to 2 mL filtrated plasma sample. A microfluidic separation system with a focusing channel of 7 μm width, 120 μm length, 50 μm depth, and volume split ratio of 29:1 (O2:O1) was mounted on an epifluorescence microscope (Nikon® Eclipse Ti) with a 20× optical objective. After priming with 1×PBS, the solution was injected at 60 μL/min using a syringe pump. In FIG. 25A, the 530 nm red microspheres experienced significant wall lift force and the flow trajectories were mostly focused along the centerline in the expansion region. The 50 nm particles experienced weaker wall lift force and spanned across the channel width. Furthermore, the sample from the O1 and O3 outlets was almost free from 530 nm red microspheres.

FIG. 25B illustrates fluorescent images of samples of carrier fluid taken at an inlet and at different outlets in the microfluidic separation system of FIG. 25A. A sample of 12 microliters was taken from the side channel outlets O1 and O3 and a sample of 300 microliters was taken from the sorter outlet O2. The samples were imaged with Cellometer chamber slides from Nexcelom Biosciences LLC under 20× optical objective with an epifluorescence microscope (Nikon® Eclipse Ti) The side channel outlet samples contained minimal 530 nm red microspheres. Fluorescence intensities of the 50 nm green microspheres were similar in the side channel outlet samples and the sorter outlet sample.

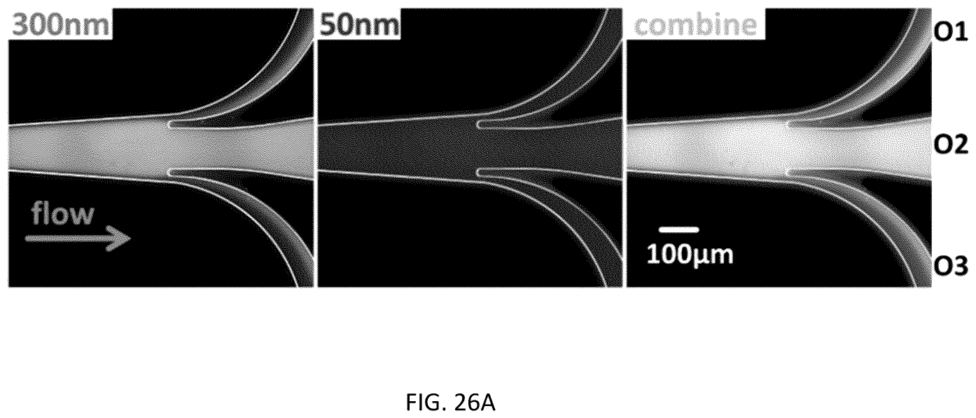

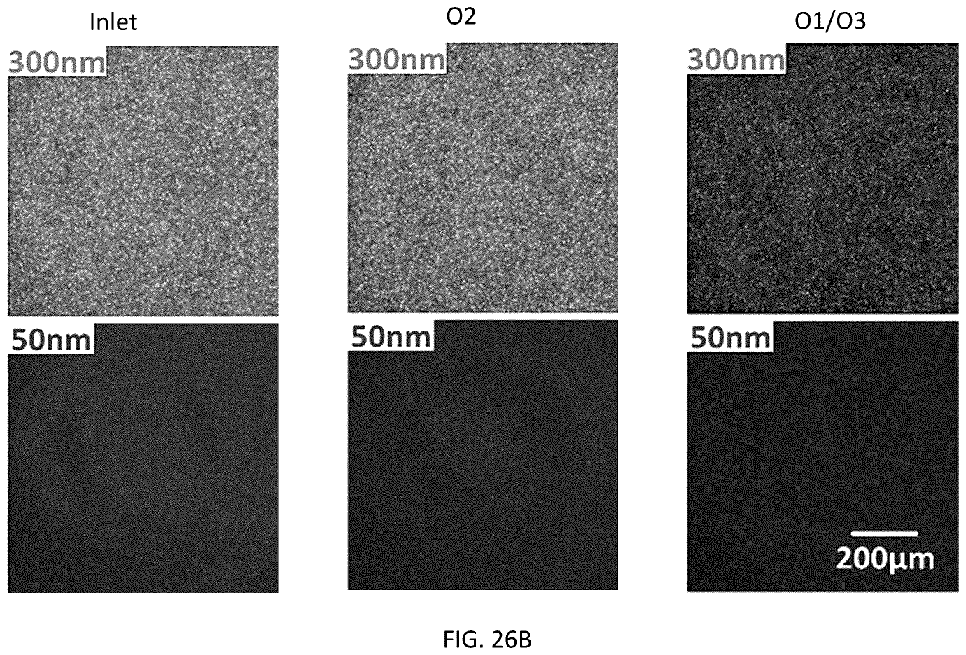

FIG. 26A shows the separation of a first group of particles having a size of 300 nm and a second group of particles having a size of 50 nm in an expansion region of a microfluidic separation system having a focusing channel (e.g., a sorter constricted region) of 5 micrometer width, 120 micrometer length, and 50 micrometer height at a carrier fluid flow rate of 40 μL/min and a volume split ratio of 29:1 (O2:O1). As illustrated, the sorter has side channel outlets O1 and O3 and a downstream outlet (e.g., a sorter outlet) O2.

Specifically, the first group of particles were Fluoresbrite® YG Carboxylate Microspheres 0.30 μm (300 nm) and the second group of particles were Fluoresbrite® YO Carboxylate Microspheres 0.05 μm (50 nm). Thus, the microspheres used may mimic separation of first biological particles having a 300 nanometer diameter and second biological particles having a 50 nanometer diameter.

Despite a small portion of 300 nm YG microspheres being unintentionally leaked into the side outlet channels, the sample collected from the O1 and O3 outlets was mostly free from 300 nm green microspheres as illustrated in FIG. 26B. FIG. 26B illustrates fluorescence images of samples of carrier fluid taken at an inlet and at different outlets in the microfluidic. Samples of 40 microliters were taken from the side channel outlets O1 and O3 and a sample of 1.2 milliliters was taken from the sorter outlet O2.

The fluorescence intensity of each image in FIGS. 25B and 26B were processed to determine the average intensity ratio of the outlet samples with the inlet sample as shown in Table 1 below.

TABLE 1
Summary of Intensity Ratio (IR) of Sample Fluorescent
Images of FIG. 25B and FIG. 26B.

| | Focusing Channel of 7 μm | | | Focusing Channel of 5 μm | | |
|---|---|---|---|---|---|---|
| | width at 60 μL/min | | | width at 40 μL/min | | |
| Outlet | | | 50 nm | | | 50 nm |
| Sample/Inlet | 530 | 50 nm | IR/530 | 300 | 50 nm | IR/300 |
| Sample | nm IR | IR | nm IR | nm IR | IR | nm IR |
| O2/Inlet | 1.21 | 1.19 | 0.98 | 0.88 | 0.88 | 1.00 |
| (O1O3)/Inlet | 0.26 | 0.77 | 2.96 | 0.35 | 0.70 | 2.00 |

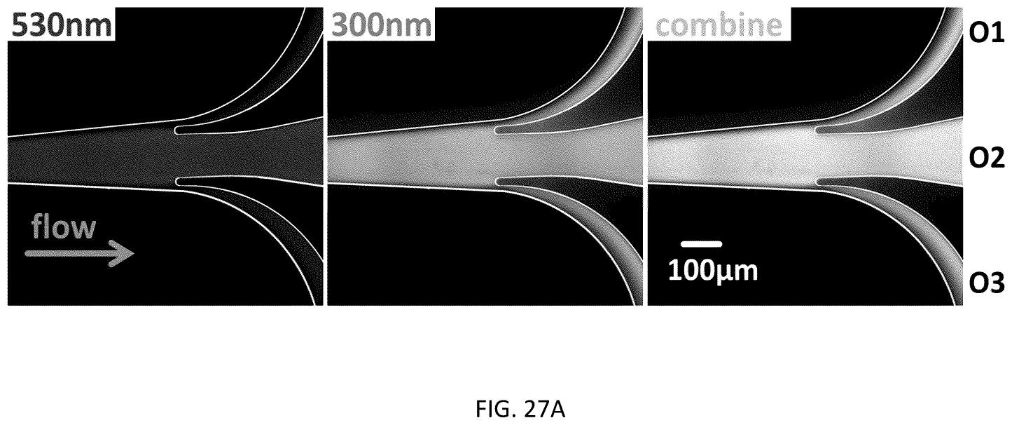

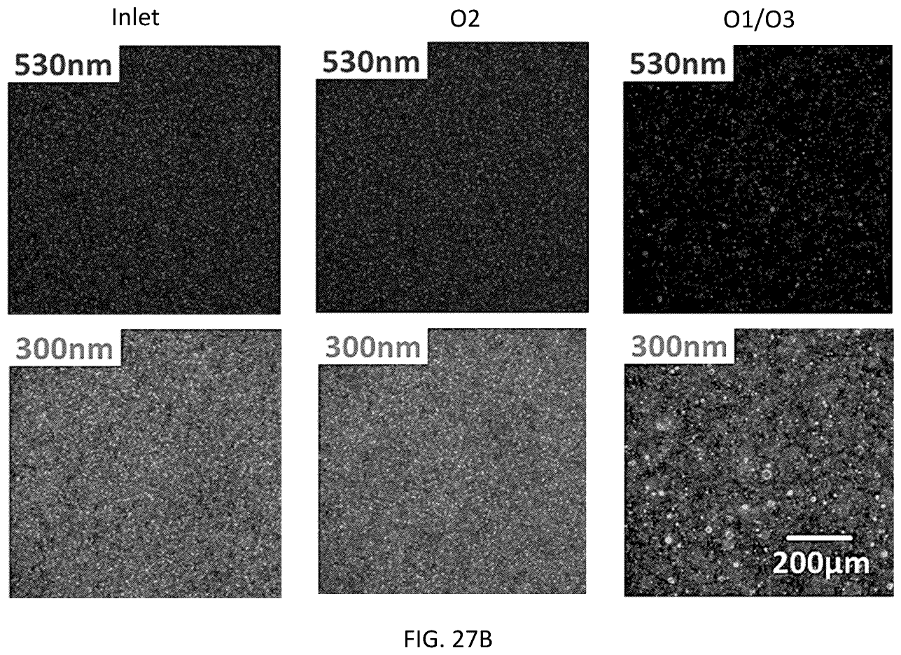

FIG. 27A shows the separation of a first group of particles having a size of 530 nm and a second group of particles having a size of 300 nm in an expansion region of a microfluidic separation system having a focusing channel (e.g., a sorter constricted region) of 7 micrometer width, 120 micrometer length, and 50 micrometer height at a carrier fluid flow rate of 40 μL/min and a volume split ratio of 24:1 (O2:O1). As illustrated, the sorter has side channels O1 and O3 and a downstream outlet (e.g., a sorter channel) O2.

Specifically, the first group of particles were SPHERO™ Fluorescent Nile Red Microspheres, 0.53 μm (530 nm) and the second group of particles were Fluoresbrite® YG Carboxylate Microspheres 0.30 μm (300 nm). Thus, the microspheres used may mimic separation of first biological particles having a 530 nanometer diameter and second biological particles having a 300 nanometer diameter.

As illustrated in FIG. 27A, the 530 nm particles illustrated tighter inertial focus compared with the 300 nm particles. The fluorescence images illustrated in FIG. 27B also illustrated tighter inertial focus as the images of the side channel outlet sample (O1/O3) illustrated the sample was mostly free from of the 530 nm particles.

The fluorescence intensity of each image in FIG. 27B was processed to determine the average intensity ratio of the outlet samples with the inlet sample as shown in Table 2 below.

TABLE 2
Summary of Intensity Ratio (IR) of Sample Fluorescent
Images of FIG. 27B

| Outlet Sample/ | Focusing Channel of 7 μm width at 40 μL/min | | |
|---|---|---|---|
| Inlet Sample | 530 nm IR | 300 nm IR | 300 nm IR/530 nm IR |
| O2/Inlet | 1.05 | 1.00 | 0.95 |
| (O1O3)/Inlet | 0.39 | 0.75 | 1.94 |

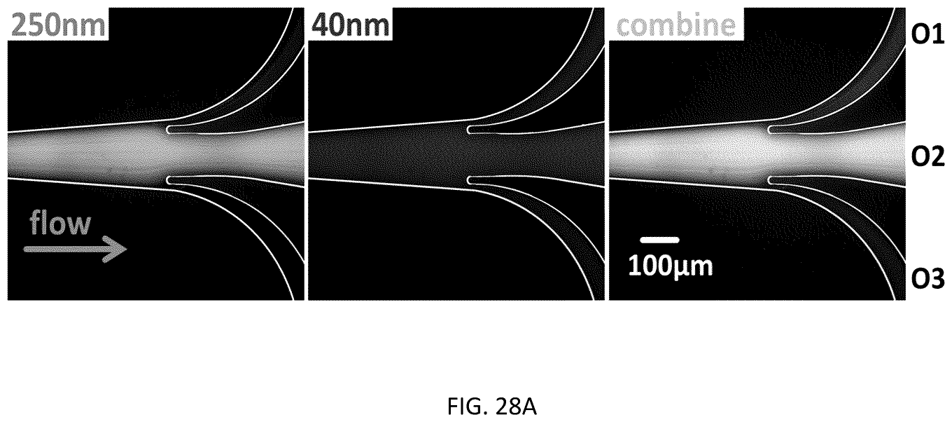

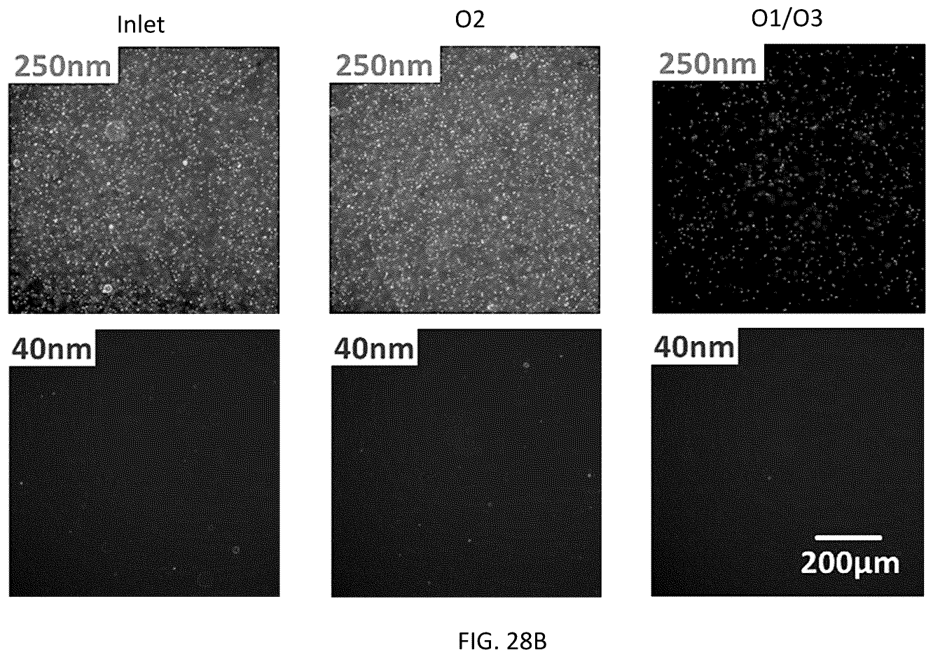

FIG. 28A shows the separation of a first group of particles having a size of 250 nm and a second group of particles having a size of 40 nm in an expansion region of a microfluidic separation system having a focusing channel of 5 micrometer width, 120 micrometer length, 50 micrometer height, and a volume split ratio of 24:1 (O2:O1) at a carrier fluid flow rate of 40 μL/min. As illustrated, the sorter has side channel outlets O1 and O3 and a downstream outlet (e.g., a sorter outlet) O2.

Specifically, the first group of particles were 250 nm Green fluorescent microspheres and the second group of particles were 40 nm Nile Red fluorescent microspheres. Thus, the microspheres used may mimic separation of first biological particles having a 250 nanometer diameter and second biological particles having a 40 nanometer diameter.

The flow trajectories of 250 nm particles were mostly diverted into the O2 sorter outlet by increasing the fluidic resistance in the O1 and O3 side channel outlets. The 40 nm particles remained dispersed across the microfluidic channel width in the expansion region. By increasing the resistance in the O1 and O3 side channel outlets, the purity of the 40 nm particle carrier fluid collected at the O1 and O3 side channel outlets was improved.

FIG. 28B illustrates fluorescent images of samples of carrier fluid taken at an inlet and at different outlets in the microfluidic separation system of FIG. 28A. The fluorescence intensity of each image in FIG. 28B was processed to determine the average intensity ratio of the outlet samples with the inlet sample as shown in Table 3 below.

TABLE 3
Summary of Intensity Ratio (IR) of Sample Fluorescent
Images of FIG. 28B

| Outlet Sample/ | Focusing Channel of 5 μm width at 40 μL/min | | |
|---|---|---|---|
| Inlet Sample | 250 nm IR | 40 nm IR | 40 nm IR/250 nm IR |
| O2/Inlet | 1.45 | 1.10 | 0.76 |
| (O1O3)/Inlet | 0.16 | 0.27 | 1.62 |

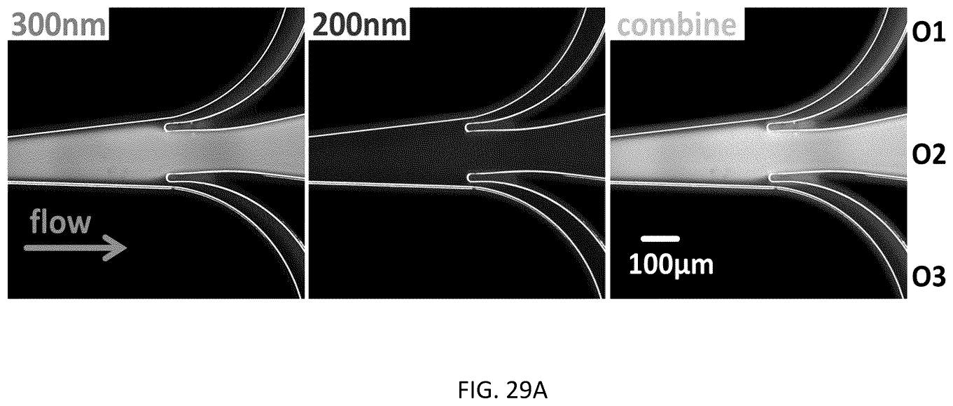

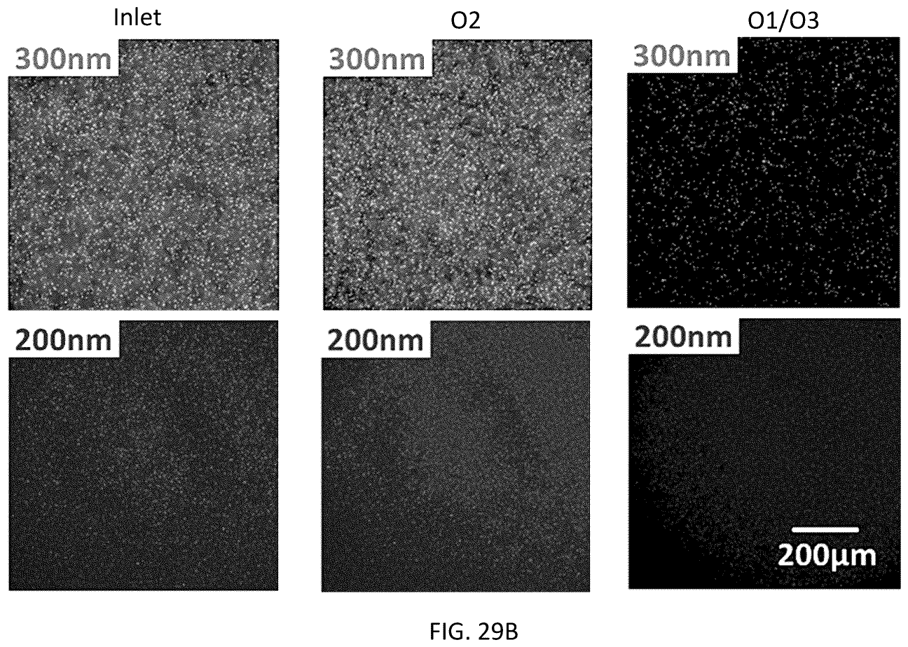

FIG. 29A shows the separation of a first group of particles having a size of 300 nm and a second group of particles having a size of 200 nm in an expansion region of a microfluidic separation system having a focusing channel of 5 micrometer width, 120 micrometer length, 50 micrometer height, and a volume split ratio of 24:1 (O2:O1) at a flowrate of 40 μL/min.

Specifically, the first group of particles were 300 nm YG fluorescent microspheres and the second group of particles were 200 nm YO fluorescent microspheres. Thus, the microspheres used may mimic separation of first biological particles having a 300 nanometer diameter and second biological particles having a 200 nanometer diameter.

Inlet and outlet samples were taken and imaged as illustrated in FIG. 29B. The fluorescence intensity of each image in FIG. 29B was processed to determine the average intensity ratio of the outlet samples with the inlet sample as shown in Table 4 below. As illustrated in Table 4, the average intensity of 200 nm particles was about 2.5 fold of the average intensity of the 300 nm particles in the O1/O3 side channel outlets.

TABLE 4
Summary of Intensity Ratio (IR) of Sample Fluorescent
Images of FIG. 29B

| Outlet | Focusing Channel of 5 μm width at 40 μL/min | | |
|---|---|---|---|
| Sample/ | | | 200 nm IR/ |
| Inlet Sample | 300 nm IR | 200 nm IR | 300 nm IR |
| O2/Inlet | 0.99 | 1.18 | 1.19 |
| (O1O3)/Inlet | 0.23 | 0.56 | 2.47 |

FIG. 30A shows the separation of a LUZ19 Phage particles and FITC conjugated LPS endotoxins in an expansion region of a microfluidic separation system having a focusing channel of 10 micrometer width, 1 centimeter length and volume split ratio 3.3:1 (O2:O1) at a carrier fluid flow rate of 40 μL/min.

The carrier fluid was injected into the inlet by a syringe pump. The flow trajectories of the LUZ19 Phage particles, the LPS endotoxins, and the combined solution is shown in FIG. 30A. A portion of the LUZ19 Phage particles was lifted by the side wall and the FITC-LPS endotoxins spanned across the channel. A thin endotoxin band free from LUZ19 phage particles was collected by side channel outlets O1 and O3.

The O2 sample was collected and reinjected into the inlet of the same device at the same flow rate for continuous separation. FIG. 30B illustrates the flow trajectories of LUZ19 phage particles and FITC-LPS endotoxins at the fourth recirculation cycle. The sorter outlet O2 sample and the side channel outlet O1 and O3 samples were collected from four microfluidic separation cycles, which illustrated that the endotoxin/phage ratio of O1 O3 samples were 12% to 26% higher than the O2 sample based on fluorescence plate readings.

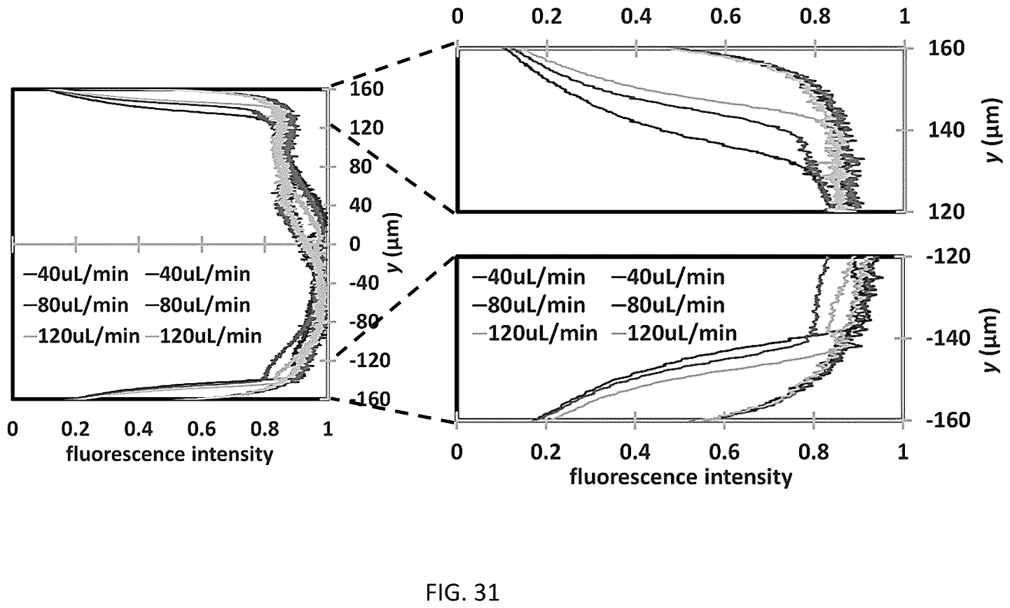

FIG. 31 shows intensity profiles along the A-A′ line illustrated in FIG. 30A for the LUZ19 phage particles and the FITC conjugated LPS endotoxins in the expansion region at flow rates of 40 μL/min, 80 μL/min, and 120 μL/min. The graph on the left indicates the overall fluorescence intensity profiles for the phage particles at the various flow rates and the fluorescence intensity profiles for the endotoxins at various flow rates. The upper-right graph is a magnified portion of the intensity profiles nearest to the left sidewall and the lower-right graph is a magnified portion of the intensity profiles nearest to the right side wall. Table 5 below shows the cross-channel position at which the fluorescence intensity of the phage flow trajectory profile reached 70% of the peak intensity. The portion of channel span to reach 70% from each half of the microfluidic channel width was between 0.09 and 0.17 at flow rates from 40 μL/min to 200 μL/min.

TABLE 5
Characterization of Cross-Channel Position at
which the Fluorescent Intensity of LUZ19
Phage Profile reached 70% of Peak Intensity

| Flow | Position to reach | Portion of Channel Span to |
|---|---|---|
| Rate | 70% of Peak | reach 70% from Each |
| (μL/min) | Intensity (μm) | Half of the Channel |
| 40 | 28 [left]; 20 [right] | 0.17 [left]; 0.12 [right] |
| 60 | 25 [left]; 19 [right] | 0.16 [left]; 0.12 [right] |
| 80 | 20 [left]; 18 [right] | 0.12 [left]; 0.11 [right] |
| 100 | 17.5 [left]; 16 [right] | 0.11 [left]; 0.10 [right] |
| 120 | 16 [left]; 15 [right] | 0.10 [left]; 0.09 [right] |
| 160 | 15.5 [left]; 15 [right] | 0.10 [left]; 0.09 [right] |
| 200 | 15.5 [left]; 15 [right] | 0.10 [left]; 0.09 [right] |

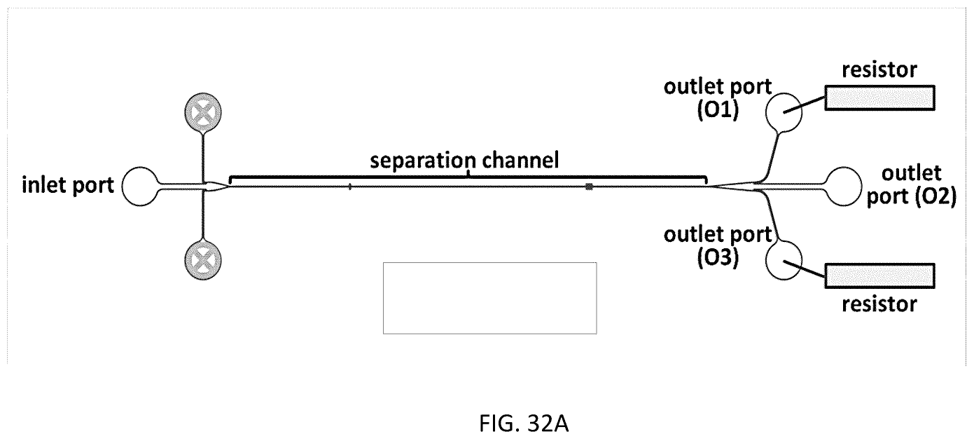

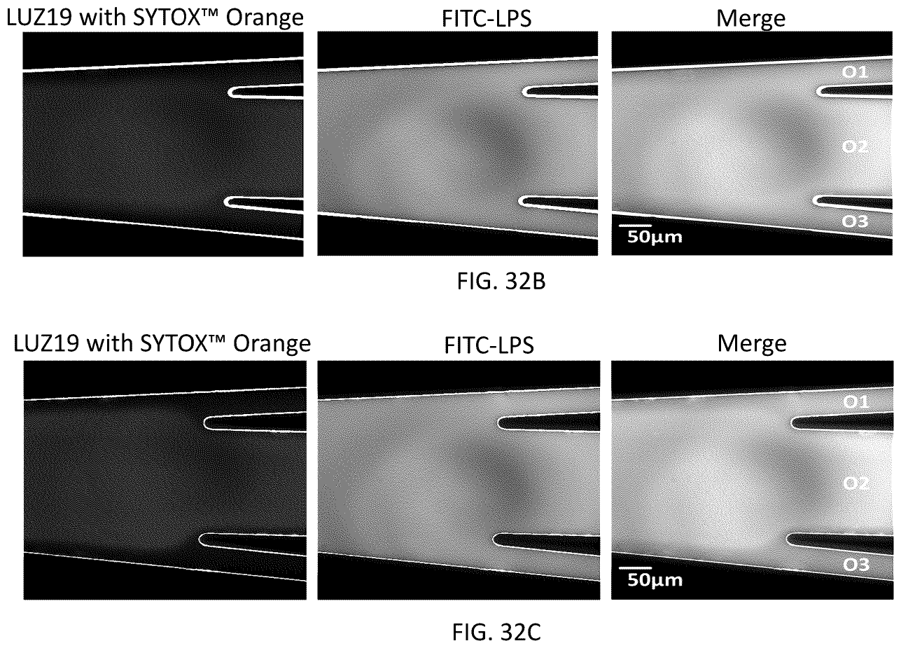

FIG. 32A illustrates a configuration of a microfluidic separation system in which fluidic resistance in the sorter side outlets (O1, O3) is manipulated by resistors to alter separation efficiency. Specifically, different lengths of 100 micrometer inner diameter PEEK tubing were inserted into side channel outlets O1 and O3 to manipulate increased outlet fluidic resistance.

FIG. 32B shows the separation of a LUZ19 Phage particles and FITC conjugated LPS endotoxins in the expansion region of the microfluid separation system of FIG. 32A having a focusing channel of 10 micrometer width, 2 centimeter length and volume split ratio 3.3:1 (O2:O1) at a carrier fluid flow rate of 20 μL/min in which 100 micrometer ID PEEK tubes with 4.5 cm length were inserted into the sorter side outlets (O1, O3).

In FIG. 32B, the carrier fluid including the phage particles and the endotoxins was injected into the inlet of the microfluidic separation system via a syringe pump. The flow trajectories of the phage particles and the FITC conjugated LPS endotoxins at the expansion region were imaged with an epifluorescence microscope (Nikon® Eclipse Ti). A portion of the phage particles were lifted by the side wall and the endotoxins spanned across the channel width. A thin endotoxin band free from phage particles was present near the sidewall in both the O1 and O3 side channel outlets.

In FIG. 32C, the length of PEEK tubes were increased to 16 centimeters and the flow rate was increased to 40 L/min. The LPS endotoxins still spanned across the channel width. Most phage particles were collected by the sorter outlet O2 due to the increased fluidic resistance in the side channel outlets O1 and O3 because of the increased length of PEEK tubes used. Thus, the carrier fluid collected in the side channel outlets O1 and O3 were nearly free from phage particles.

The fluorescence of the O2 and O1, O3 samples were measured with a microplate reader. A slight decrease in the LPS/LUZ19 level between 6% and 7% in the O2 outlet sample was measured with outlet recovery ratios of 1:8.5 and 1:20 (O1O3:O2). The LPS/LUZ19 level in the O1O3 sample was significantly increased by 270% with an outlet recovery ratio of 1:20, resulting in 75% separation efficiency, which may be determined by the following formula:

$$Sep . Eff . = 1 - \frac{{[ LPS / LUZ 19 ]}_{O 2}}{{[ LPS / LUZ 19 ]}_{O 1 O 3}}$$

The improvement in separation efficiency by adding resistance to the side channel outlets O1 and O3 using the PEEK tubes in the outlets is shown in Table 6 below.

TABLE 6
Improvement in Separation Efficiency of LUZ19
Phage particles and FITC-LPS Endotoxins
with Different Outlet Recovery Ratios

| | Inlet | | | | LPS/ |
|---|---|---|---|---|---|
| | Flow | Outlet | LPS/ | LPS/ | LUZ19 |
| | Rate | Recovery | LUZ19 | LUZ 19 | (Sep. |
| Configuration | (μL/min) | (O1O3:O2) | (O2) | (O1, O3) | Eff %) |
| L = 1 cm; W= 10 | 40 | 1:4 | ↑ 6% | ↑ 40% | 26% |
| μm | | | | | |
| L = 2 cm; W= 10 | 20 | 1:8.5 | ↓ 7% | ↑ 60% | 40% |
| um (4.5 cm PEEK | | | | | |
| tube) | | | | | |
| L = 2 cm; W= 10 | 40 | 1:20 | ↓ 6% | ↑ 270% | 75% |
| μm (16 cm PEEK | | | | | |
| tube) | | | | | |

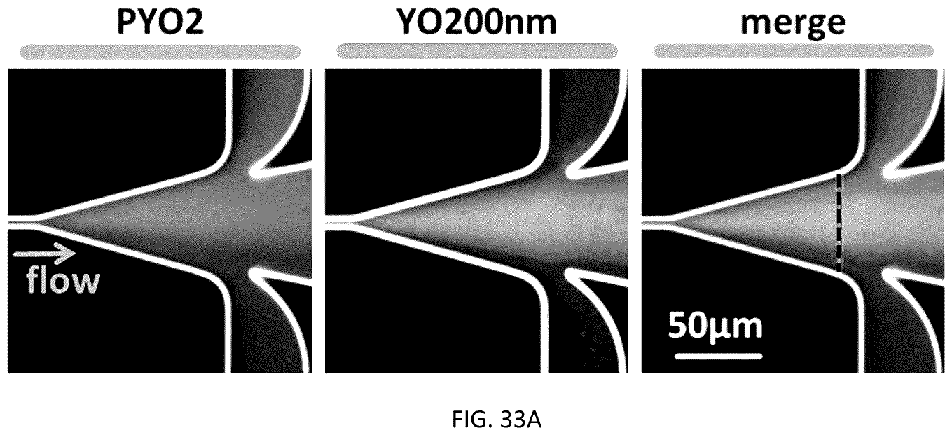

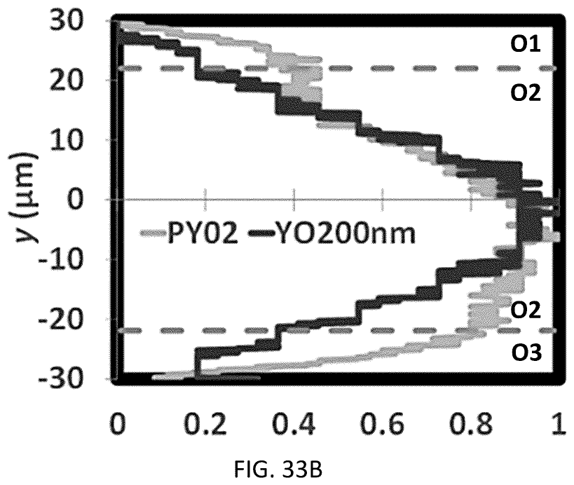

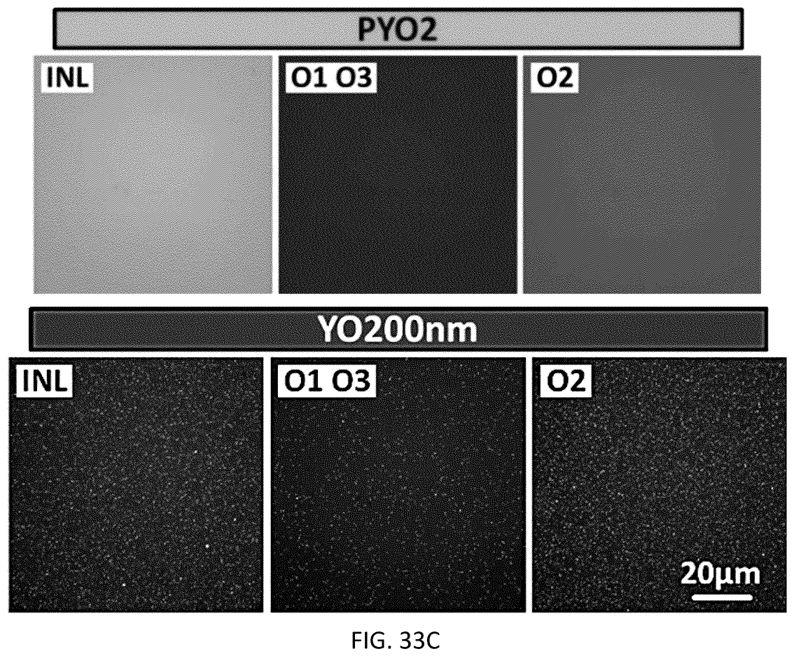

FIG. 33A shows the separation of a first group of PY02 phage particles from a second group of YO nanoparticles having a size of 200 nm in an expansion region of a microfluidic separation system having a focusing channel with 10 micrometer length, 14 micrometer height with an expansion angle of 30 degrees from the focusing channel at a carrier fluid flow rate of 5 μL/min.

Specifically, the first group of particles were fluorescently labeled PYO2 phage particles and YO200 nm fluorescently labeled nano-particles. Phage PYO2 is a podovirus with a 72 nm diameter icosahedral head and a 18 nm short tail.

The carrier fluid was driven through the system using a syringe pump. Given the small-size of Phage PYO2, the PYO2 phage particles were briefly focused by inertial force and thus, spanned across the entire channel width. Most PYO2 phage particles were collected by the centerline outlet O2, leaving a small fraction of phage-free liquid phase near the far end of the side outlet (O1 & O3) walls. In contrast, the larger YO200 nm nano-particles showed significantly tighter focusing due to stronger inertial force by the microfluidic focusing channel and were collected by the center outlet O2. Only a small fraction of the nanoparticles were retrieved from the side outlets O1 and O3.

FIG. 33B illustrates the intensity profile along the A-A′ line illustrated in FIG. 33A for the PY02 phage particles and the YO 200 nm nanoparticles. The smaller PYO2 phages have wider spreading with full-width-half-maximum (FWHM) of 61 μm. With larger diameter, the FWHM of YO200 nm nano-particles was measured at 40 μm.

FIG. 33C illustrates fluorescent images of samples of carrier fluid taken at an inlet and at different outlets in the microfluidic separation system of FIG. 33A to determine the intensity level for PYO2 and particle counts for YO 200 nm. The center line outlet O2 had 88% YO 200 nm particle count compared with 72% for PYO2 in average fluorescence intensity measurement. Table 7 below shows the separation performance summary for the PYO2 phage and YO200 nm fluorescent nano-particles solution.

TABLE 7
Separation Performance Summary for PYO2 phages and
YO 200 nm fluorescent nano-particles.

| PYO2 (Fluorescence Intensity) | | | YO200 nm (Particle Count) | | |
|---|---|---|---|---|---|
| INL | O1 O3 | O2 | INL | O1 O3 | O2 |
| 82 | 16 (28%) | 41 (72%) | 8520 | 1343 (12%) | 10590 (88%) |

FIG. 34A illustrates a microfluidic separation system utilizing multiple carrier fluid sorters in series that may be used to separate phage particles from bacteria. As illustrated in FIG. 34A, the carrier fluid may include bacteria, which may be first biological particles, and phage particles, which may be second biological particles. The separation system may be configured similarly to the microfluidic separation systems described throughout this disclosure. In operation, the carrier fluid may enter the inlet of the microfluidic separation system. The sorters may be configured to separate the bacteria from the phage particles. For example, a first fraction of carrier fluid that is bacteria-poor and phage-concentrated may be directed to the at least one sorter side channels, and a second fraction of carrier fluid that is bacteria-concentrated and phage-poor may be directed to the sorter outlet (e.g., the downstream outlet). As illustrated, each sorter side channel may be fluidly coupled with a permeate outlet and each sorter outlet may be coupled with a retentate outlet. Thus, the first fraction of carrier fluid may be directed to the permeate outlet and the second fraction of carrier fluid may be directed to the retentate outlet.

In some embodiments, the height of the sorter side channels may be less than a height of the sorter outlet to increase fluidic resistance provided by the at least one sorter side channel such that the first fraction of the carrier fluid is directed to the at least one sorter side channel and the second fraction of the carrier fluid is directed to the at least one sorter outlet.

The microfluidic separation system shown in FIG. 34A may be a branch of a microfluidic plate having 36 radially arranged branches connected to the inlet. Each branch may be made up of 30 micrometer wide focusing channels interfaced with expansion regions and each microfluidic branch may have five serially connected sorter units.

In some embodiments, the sorter constricted region (e.g., the focusing channel) may be dimensioned to direct the first fraction to the sorter side channels and the second fraction to the at least one sorter side channel. For example, the focusing channel may have a width of about 15 micrometers, a length between about 3 millimeters and about 4 millimeters, and/or a height of about 60 micrometers. In some embodiments, the width may be between about 7 micrometers and about 20 micrometers, the length may be between about 0.2 millimeters and about 4 centimeters, and/or the height may be between about 40 micrometers and about 500 micrometers. In these and other embodiments, a flow rate of 200 μL/min may be used. In some embodiments, the flow rate may be between about 20 microliters per minute and about 1 milliliter per minute.

The microfluidic separation system illustrated in FIG. 34A was tested with carrier fluid including contaminant bacteria and phage particles. Bacillus subtilis strain NRRL-571 was used as the bacterial contaminant for this test. An overnight culture was added to fresh Luria-Bertani (LB) broth and cultured to OD600=0.1. The bacterial solution was mixed with PYO2 phage solution at 5×107 pfu/mL and driven into system at 6 mL/min using a syringe pump (Harvard Apparatus, MA). Phage PYO2 is a podovirus with a 72 nm diameter icosahedral head and an 18 nm short tail [2]. The filtered sample was collected from the “permeate” outlet. The spot-titer assay results showed that 5×10⁷ pfu/mL of phage PYO2 was recovered from the “permeate” samples. These data show that phage concentration was not significantly reduced compared to the starting sample, and the microfluidic filtration procedure did not negatively impact phage lytic activity as illustrated in FIG. 34B.

The system was tested to continuously filter ˜7 liters of the carrier fluid in a closed-loop configuration to demonstrate membrane-less filtration. No clogging issues were encountered in contrast to dead-end membrane filtration, in which contaminant particulates or bacterial debris can be physically trapped in membrane pores and prohibit filtration in large volume or high contaminant content. An inlet port was connected to the source reservoir (1-liter reagent flask) containing B. subtilis bacterial solution (OD600=0.1) via an OEM peristaltic pump (Instech, P625/66.xxx) at an input volume flow rate of 6 mL/min. The “retentate” outlet was connected back to the source reservoir for re-circulation. The “permeate” outlet was connected to another reagent flask to collect filtrated solution. The entire experimental setup was immersed in ice to suppress bacterial growth. About 200 mL of filtrated solution was extracted without clogging issues during the 24-hour collection period. The filtered sample measured about an 80% decrease in OD600 value compared with the inlet sample. Agar plate culture results showed about 2-log order reduction in CFU/mL value in the filtered samples.

The system was tested interfacing with a conical flask [BR2] to characterize filtration and phage recovery efficiencies. The [BR1] culture was prepared in a 150 ml conical flask by overnight culture of 50 mL PAO1 WT bacterial solution in LBNS at 30° C. with a 100 rpm shaker. Due to bacterial overgrowth, a 40 μm inline strainer (Pluriselect usa, Inc.) was used to remove large clumps in the bacterial solution before diluting down to OD600 value 0.2 in a 500 mL [BR2] conical flask with 150 mL fresh LBNS. A 100 μL E215 phage stock solution (at 1.5×1011 pfu/mL) prepared by Roach Lab was added into the bacterial solution which gave an initial MOI 0.1 at 1×108 pfu/mL. The [BR2] bacterial liquid culture inoculated with E215 phage was incubated for 6 hours at 30° C. under 100 rpm shaking. The [BR2] conical flask was connected with the system which was primed with 1×PBS with the Kamoer peristaltic pump (KXF-DC-C06). In this experimental setup, the retentate outlet was connected to the [BR2] conical flask to enable multi-pass recirculation operation. The [BR2] was kept in ice to slow down bacterial growth. The power level of the peristaltic pump was set at 40% and the system was ran continuously for 2 hours. 1 mL solution from the [BR2] flask was collected after 5 min of operation to minimize dilution effect in the permeate sample. Bacterial, phage and endotoxin concentrations were measured every 30 min, including the starting and endpoint samples from the [BR2] liquid culture. For every sample, the PAO1 WT bacterial concentration was first measured by photospectrometer (VARIAN Cary 50 Bio) in OD600 using a new cuvette. 400 μL of the bacterial solution was transferred from the cuvette into two 1.5 mL microcentrifuge tubes. After pelleting bacteria by centrifugation at 8,000 g for 5 min, 300 μL supernatant samples were collected and stored overnight in the fridge at 4° C. In the following day, the E215 phage concentration was measured by phage titration (spotted method) and LPS endotoxin concentration by a Chromogenic Endotoxin Quant kit [1]. The phage recovery and bacterial filtration results are summarized in FIG. 35 and Table 8. The bacterial concentration was kept roughly less than 1% in the first 30 min of operation. Both the phage titer and endotoxin level in the filtrated samples maintained similar value in the permeate sample in comparison with the [BR2] sample. The leftmost graph illustrates the pfu/mL measurement of phage concentration, the middle graph illustrates EU/mL measurement of endotoxin concentration, and the right graph illustrates OD600 measurement of bacteria concentration. As illustrated in FIG. 35, the permeate sample is bacteria poor, but phage concentrated indicating the ability of the system to separate bacteria and phage particles.

TABLE 8
Phage Recovery and Bacterial Filtration Performance

| | Inlet ([BR2] conical flask) | | Permeate Outlet | | |
|---|---|---|---|---|---|
| Particle | Starting | 2 hr | | 30 min | 1 hr |
| Type | sample | sample | Start | sample | sample |
| Phage | 1.3 × 10¹⁰ | 1.0 × 10¹⁰ | 1.5 × 10¹⁰ | 1.3 × 10¹⁰ | 7.5 × 10¹⁰ |
| (E215) | | | | | |
| (pfu/mL) | | | | | |
| Endotoxin | 3.9 × 10⁵ | 3.7 × 10⁵ | 2.9 × 10⁵ | 3.2 × 10⁵ | 3.1 × 10⁵ |
| (LPS) | | | | | |
| (EU/mL) | | | | | |
| Bacteria | 0.748 | 0.776 | 0.005 | 0.010 | 0.028 |
| (PAO1) | | | | | |
| (OD₆₀₀) | | | | | |

Table 9 below illustrates the bacterial filtration performance using a focusing channel of 7 μm width, 120 μm length and 50 μm, and outlet volume splitting ratio of 29:1 (O2:O1) in conjunction with the system illustrated in FIG. 34A. A Pseudomonas aeruginosa bacterial solution (PAO1 WT) in LBNS was prepared by overnight culture under 200 rpm shaking at 30° C. The PAO1 bacterial solution was subcultured to OD600 0.006 the following day. The bacterial concentration was measured by optical absorbance at 600 nm with a microplate reader (Molecular Devices Spectramax 250) after background subtraction of LBNS. The system was connected to a digital variable-speed multichannel peristaltic pump (Ismatec™ MS-4/12 Reglo Digital Pump) to demonstrate bacterial concentration. A pump tubing with 1.30 mm ID was used (Masterflex® Ismatec® Pump Tubing, 3-Stop, Tygon® S3™ E-Lab). The system was first primed with 1×PBS. An input of the peristaltic pump tubing was connected from a 15 ml conical tube containing 1×PBS to the inlet of the system. The 1×PBS was pumped through the device at a flow rate setting of 0.2 for priming. The O2 outlet tubing was clipped and the flow rate was reduced to 0.111 (minimal speed) to prime the remaining O1 and O3 outlets. The input tubing of the peristaltic pump was placed to the bottom of a 15 mL conical tube containing 2 mL PAO1 bacterial solution. The O2 outlet tubing was also inserted into the bacterial solution for continuous recirculation operation. The O1 and O3 outlet tubing was inserted into another 15 ml conical tube for filtrate collection. The flow rate of the peristaltic pump was set at 5.00 and ran for 2.5 hours. The bacterial concentration result based on OD600 measurement was summarized in Table 9. The PAO1 WT bacterial solution was concentrated to OD600 0.026 (6.5×) from an initial concentration of OD600 0.004. The initial bacterial solution concentration reading was adjusted from OD600 0.006 to account for the fill-in volume dilution with 1×PBS during the priming process. The bacterial solution volume was reduced from 2 mL to 170 μL. The bacterial concentration of the O1 and O3 filtrate was OD600 0.002.

TABLE 9
Phage Recovery and Bacterial Filtration Performance

| Measurement | Start (O2) | End (O2) | O1 O3 Sample |
|---|---|---|---|
| OD₆₀₀ | 0.004 | 0.026 | 0.002 |
| Volume | 2 mL | 170 μL | 1.8 mL |

As previously discussed, the microfluidic separation system may be utilized to separate and/or concentrate other biological particles such as liposomes, EVs, exosomes, and/or cell-free DNAs. For example, the microfluidic network 1900 may be used to separate liposomes from 16-nucleotide DNA molecules. In these and other embodiments, the first biological molecules may be liposomes, EVs, or exosomes, and the second biological molecules may be nucleic acids (e.g., Cell-free DNAs), proteins, or polypeptides. In these and other embodiments, the sorter constricted region may have a width of about 5 micrometers, a length of about 120 micrometers, and/or a height of about 50 micrometers. In some embodiments, the sorter constricted region may have a width between about 2 micrometers and about 10 micrometers, the length may be between about 0.2 millimeters and about 2 centimeters, and/or the height may be between about 10 micrometers and about 250 micrometers. As an example, FIG. 36A shows the separation of liposomes and 16-nucleotide molecules in an expansion region of a microfluidic separation system having a focusing channel of 5 micrometer width, 120 micrometer length and 50 micrometer height at flow rates of 40 μL/min and 80 μL/min. In some embodiments, the flow rate may be between about 2 μL/min and about 500 μL/min

Specifically, synthetic liposomes were generated which may mimic medium or small sized EV and 16 nucleotide DNA molecules were used which may mimic cell-free miRNAs to demonstrate the use of the microfluidic system to separate EVs from cell-free RNAs. The liposomes were generated by lipid film hydration method, followed with sonication. Three natural lipids were purchased from Avanti Polar Lipids, Inc.: (1) DSPC: 1,2-distearoyl-sn-glycero-3-phosphocholine, 18:0 PC (Catalog #: 850365), (2) Chol: cholesterol, derived from plant (Catalog #: 700100), (3) PE-CF (fluorescent): 1,2-dioleoyl-sn-glycero-3-phosphoethanolamine-N-(carboxyfluorescein) (ammonium salt), 18:1 PE CF (Catalog #: 810332). 110 mL of 25 mg/mL DSPC and 15 mL of 10 mg/mL Chol in chloroform were added into a 20 mL glass bottle for liposome preparation with molar ratio of 90:10 (DSPC: Chol). The glass bottle was laid, gently rotated, and placed on a hot plate at 45° C. for chloroform evaporation in a chemical hood. After vacuuming in a desiccator overnight to remove remaining chloroform, 3 mL 1×PBS was added to the dry lipid film for hydration in the following day. The aqueous liposome solution was first vortexed at maximum speed to resuspend the lipid materials, followed with sonication in water bath for >1 hr. The liposome suspensions were stored at 4° C. overnight to hydrate remaining lipid materials.

1 mL of the liposome solution was fluorescently labeled with 5 μM Vybrant™ DiO Cell-Labeling Solution (Catalog #: V22886) from Invitrogen™ for >5 mins. 16 nt DNA solution was obtained from Integrated DNA Technologies (IDT) Inc. with sequence of 5′-AGG TCA AGG TAT CTC T-3′ and reconstituted at 1.2 nM in nuclease-free water. 200 μL of the 16 nt DNA solution was stained in 10 μM SYTOX™ Orange (Catalog #: S34861) obtained from Thermo Fisher Scientific® Inc. The solutions were mixed without additional washing or filtration.

Microfluidic separation was demonstrated with an focusing channel of 5 μm width, 120 μm length, 50 μm depth, and volume split ratio of 29:1 (O2:O1) at 40 μL/min and 80 μL/min. In FIG. 36A, the synthetic liposomes experienced significant lift force from side walls. Liposomes were collected by centerline O2 outlet channel and no liposome was leaked into side O1 and O3 outlet channels. The 16 nt DNA molecules experienced negligible wall lift force and spanned across the channel width. The flow rate was increased to 80 μL/min, but no significant change of flow trajectories from both liposomes and 16 nt DNA molecules was observed. The 16 nt DNA solution collected from the O1 and O3 outlet channels was mostly free from synthetic liposomes.

FIG. 36B illustrates fluorescence images of samples of the carrier fluid taken at an inlet and at different outlets in the microfluidic separation system of FIG. 36A at a flow rate of 80 μL/min. A 10 μL O1 and O3 side channel outlet sample and a 300 μL O2 outlet sample was used for imaging. In comparison with the inlet sample, the side O1 and O3 outlet sample was almost free from synthetic liposomes but still contained a considerable amount of 16 nt DNA solution. The fluorescence intensity of each image in FIG. 36B was processed to determine the average intensity ratio of the outlet samples with the inlet sample as shown in Table 9 below. As illustrated in Table 9, the fluorescence intensities of both synthetic liposomes and 16 nt DNA molecules were similar for the inlet and O2 samples. For the O1 and O3 side channel sample, the fluorescence intensity of liposomes was very low. A greater than 50 times enrichment of 16 nt/liposome ratio was demonstrated in the sample collected from the O1 and O3 outlet.

TABLE 9
Summary of Intensity Ratio (IR) of Sample Fluorescence
Images of FIG. 29B

| | Focusing Channel of 5 μm width | | |
|---|---|---|---|
| | at 80 μL/min | | |
| | | | 16nt IR |
| Outlet Sample/ | Liposome | | Liposome |
| Inlet Sample | IR | 16nt IR | IR |
| O2/Inlet | 1.07 | 1.01 | 0.94 |
| (O1O3)/Inlet | 0.006 | 0.31 | 54.68 |

A mini-extrusion kit from Avanti Polar Lipids, Inc.® was used to generate smaller liposomes to match the size range of small EVs/exosomes by membrane extrusion. Synthetic liposomes with DSPC: Chol ratio of 90:10, were prepared with dry lipid thin film hydration method followed with sonication as described above. The liposome solution was passed through the membrane of the extrusion kit back and forth over ten times to break into smaller size. Although fluorescence microscopy cannot provide quantitative measurement of liposome size, it revealed qualitative comparison between two liposome populations. The liposomes were stained in 5 μM DiO. The optical objective focus was positioned at the bottom of the imaging chamber slide where some liposomes sat on the surface and were imaged under a 20× optical objective on an epifluorescence microscope (Nikon Eclipse Ti). Without extrusion, a wider size distribution of liposomes was observed as illustrated in FIG. 37A. After membrane extrusion, the liposomes appeared to have smaller size and less size variation as illustrated in FIG. 37B. The liposomes (after sonication, without membrane extrusion) used in inertial microfluidic separation in FIG. 36A were reported to have an average size of 274 nm, while

FIG. 37C shows the separation of liposomes after membrane extrusion and 16-nucleotide molecules in an expansion region of a microfluidic separation system having a focusing channel of 5 micrometer width, 120 micrometer length, 50 micrometer height, and a volume split ratio of 24:1 (O2:O1) at flow rates of 40 μL/min and 60 μL/min. The liposome and 16 nt DNA solution was injected first at 40 μL/min. The flow trajectories of small liposomes were briefly focused at two symmetrical equilibrium positions in the expansion section. The other portion of individual liposomes with 100 nm size range experienced weak wall lift force. A considerable amount was leaked into outlet channels O1 and O3. The 16 nt DNA molecules experienced negligible wall lift force and spanned across the microfluidic channel width. Regardless, the 16 nt DNA solution collected from the O1 and O3 outlet channels was enriched at a certain degree from the liposome population. The flow rate was increased to 60 μL/min, but no significant change of flow trajectories from both fluorescent liposomes and 16 nt DNA molecules was observed. It is expected that stronger wall lift force can be exerted on small liposomes with narrower microfluidic focusing channel width, e.g., <4 μm.

FIG. 37D illustrates the intensity profiles along the A-A′ line (left) and the B-B′ line illustrated in FIG. 37C for the liposomes and 16-nucleotide molecules. At both flow rates, the profile from the extruded liposomes showed two peaks at two equilibrium positions in between the channel centerline and side walls, indicating that the liposome population contained larger aggregates. The red line profile from 16 nt DNA solution was flat, revealing negligible wall lift force. The fluorescence intensity of 16 nt DNA solution was significantly higher than the liposome population near the side walls (O1 and O3 side channel outlets).

In another test (not shown), EXOCET Exosome Quantitation Kit (Catalog #: EXOCET96A-1) from System Biosciences (SBI) was used to evaluate exosome separation efficiency of inertial microfluidics. A microfluidic separation system with a focusing channel of 7 μm width, 120 μm length, 50 μm depth, and volume split ratio of 29:1 was used to demonstrate separation of EVs in the carrier fluid. Single donor human blood (blood derived/Na citrate) plasma (Catalog #: IPLASNAC) from Innovative Research, Inc was used. After spinning down at 10,000 g in a centrifuge to remove larger blood cell components, e.g., platelets, the supernatant containing EVs was driven into the inertial microfluidic device. The EV count of the O2 sample with enriched about 30% from the O1 O3 sample. The exosome count of this human disease-free plasma without lysis buffer dilution was estimated to be at the level of 1×10¹² particles/mL.

The microfluidic separation systems described throughout this disclosure may be utilized in conjunction with particles of different sizes and/or types to separate and/or concentrate the particles. Furthermore, the microfluidic separation systems described throughout this disclosure may be used to be perform at least a portion of the methods described throughout this disclosure.

In some embodiments, a microfluidic separation system may include an inlet configured to receive a carrier fluid from a carrier fluid source, the carrier fluid including first particles. The system may include a carrier fluid sorter coupled to the inlet. The carrier fluid sorter may include a carrier fluid sorter microfluidic network. The carrier fluid sorter microfluidic network may have a sorter constricted region having a first cross-sectional dimension. The carrier fluid sorter microfluidic network may have a sorter expansion region having a second cross-sectional dimension that is larger than the first cross-sectional dimension. The carrier fluid sorter microfluidic network may include at least one sorter side channel formed into a side of the sorter expansion region. The at least one sorter side channel may be configured to receive a first fraction of the carrier fluid, the first fraction being first particle poor. The carrier fluid sorter microfluidic network may include at least one sorter outlet that is downstream or medial from the at least one sorter side channel. The at least one sorter outlet may be configured to receive a second fraction of the carrier fluid, the second fraction being first particle concentrated. The microfluidic network may be configured to direct the first fraction of the carrier fluid to the at least one sorter side channel and configured to direct the second fraction of the carrier fluid to the at least one sorter outlet.

In some embodiments, the carrier fluid may further include second particles that are smaller than the first particles, the first fraction being second particle concentrated and the second fraction being second particle poor. In some embodiments, the sorter constricted region may be defined by a width between about 2 micrometers and about 30 micrometers, a length between about 0.2 micrometers and about 2 centimeters, and a height between about 10 micrometers and about 500 micrometers. In some embodiments, a flow rate through the carrier fluid sorter microfluidic network may be between about 2 microliters per minute and about 1 milliliter per minute.

In some embodiments, the first particles may be virus particles. The carrier fluid may further include second particles that are smaller than the first particles, the first fraction being second particle concentrated and the second fraction being second particle poor. The second particles may be endotoxins or other virus particles. The sorter constricted region may have at least one of: a width between about 2 micrometers and about 10 micrometers, a length between about 0.2 millimeters and about 2 centimeters, or a height between about 10 micrometers and about 250 micrometers.

In some embodiments, the first particles may be a first group of virus particles and the second particles may be a second group of virus particles. The first group of virus particles being larger than the second group of virus particles. The sorter constricted region may have at least one of: a width between about 2 micrometers and about 10 micrometers, a length between about 0.2 millimeters and about 2 centimeters, or a height between about 10 micrometers and about 250 micrometers. The first group of virus particles may have a size between about 250 nanometers and about 530 nanometers and the second group of virus particles may have a size between about 40 nanometers and about 300 nanometers.

In some embodiments, the first particles may include bacteria. The carrier fluid may further include second particles that are smaller than the first particles, the first fraction being second particle concentrated and the second fraction being second particle poor. The second particles may be bacteriophages. A height of the at least one sorter side channel may be lesser than a height of the at least one sorter outlet to increase fluidic resistance provided by the at least one sorter side channel such that the first fraction of the carrier fluid may be directed to the at least one sorter side channel and the second fraction of the carrier fluid may be directed to the at least one sorter outlet. The sorter constricted region may have at least one of: a width between about 7 micrometers and about 20 micrometers, a length between about 0.2 millimeters and about 4 centimeters, or a height between about 40 micrometers and about 500 micrometers.

In some embodiments, the first particles may be liposomes, extracellular vesicles (EV), or exosomes. The carrier fluid may further include second particles that are smaller than the first particles, the first fraction being second particle concentrated and the second fraction being second particle poor. The second particles may be nucleic acids, proteins, or polypeptides. The sorter constricted region may have at least one of: a width between about 2 micrometers and about 10 micrometers, a length between about 0.2 millimeters and about 2 centimeters, or a height between about 10 micrometers and about 250 micrometers.

In some embodiments, the carrier fluid may be whole blood, the first particles may be red blood cells and white blood cells. The carrier fluid may include second particles that are smaller than the first particles, the first fraction being second particle concentrated and the second fraction being second particle poor. The second particles may be platelets. The sorter constricted region may have at least one of: a width between about 10 micrometers and about 30 micrometers, a length between about 0.5 millimeters and about 30 millimeters, or a height between about 25 micrometers and about 500 micrometers.

In some embodiments, the carrier fluid may be platelet-rich plasma and the first particles may be platelets. The sorter constricted region may have at least one of: a width between about 10 micrometers and about 30 micrometers, a length between about 0.5 millimeters and about 30 millimeters, or a height between about 25 micrometers and about 500 micrometers.

In some embodiments, a method of separating particles may include receiving carrier fluid at an inlet of a carrier fluid sorter, the carrier fluid including first particles. The method may include fractionating the carrier fluid into a first fraction and a second fraction, the first fraction being first particle poor and the second fraction being first particle concentrated. The method may include directing the first fraction to at least one sorter side channel of the carrier fluid sorter. The method may include directing the second fraction to at least one sorter outlet that is downstream or medial from the at least one sorter side channel.

In some embodiments, the carrier fluid may include second particles that are smaller than the first particles, the first fraction may be second particle concentrated and the second fraction may be second particle poor. In some embodiments, the first particles may be virus particles and the second particles may be endotoxins or different virus particles. The first particles may be a first group of virus particles and the second particles may be a second group of virus particles, the first group of particles being larger than the second group of particles. The first particles may be bacteria and the second particles may be bacteriophages. The first particles may be liposomes, extracellular vesicles (EV), or exosomes and the second particles may be nucleic acids, proteins, or polypeptides. The first particles may be red blood cells and white bloods cells and the second particles may be platelets. In some embodiments, the first particles may be platelets.

In some embodiments, the fractionation of the carrier fluid may be attributable, at least in part, to a sorter constricted region downstream of the inlet and upstream of the at least one sorter side channels and the at least one sorter outlet, the sorter constricted region being defined by a width between about 2 micrometers and about 30 micrometers, a length between about 0.2 micrometers and about 2 centimeters, and a height between about 10 micrometers and about 500 micrometers.

In some embodiments, the methods described throughout this disclosure may be performed by the microfluidic separation systems described throughout this disclosure.

In some embodiments, a portable platelet apheresis system can include: a whole blood inlet configured to receive whole blood from a whole blood source; an anticoagulant source containing an anticoagulant; a mixer fluidly coupled with the whole blood inlet and anticoagulant source and configured to mix the whole blood and the anticoagulant; and a microfluidic cartridge fluidly coupled with an outlet of the mixer. The microfluidic cartridge includes a whole blood sorter. The whole blood sorter has a whole blood sorter microfluidic network that includes: a sorter constricted region having a first cross-sectional dimension; a sorter expansion region having a second cross-sectional dimension that is larger than the first cross-sectional dimension; at least one sorter side channel formed into a side of the sorter expansion region; and at least one sorter outlet that is downstream or medial from the at least one side channel. A platelet poor outlet is fluidly coupled with the at least one sorter outlet. A platelet concentrator outlet is fluidly coupled with the at least one sorter side channel.

In some embodiments a portable platelet apheresis system can include a microfluidic cartridge fluidly coupled with an outlet of the mixer, wherein the microfluidic cartridge includes a whole blood sorter and a platelet concentrator downstream from the whole blood sorter. The whole blood sorter has a whole blood sorter microfluidic network that includes: a sorter constricted region having a first cross-sectional dimension; a sorter expansion region having a second cross-sectional dimension that is larger than the first cross-sectional dimension; at least one sorter side channel formed into a side of the sorter expansion region; and at least one sorter outlet that is downstream or medial from the at least one side channel. The platelet concentrator has a platelet concentrator microfluidic network that includes: a concentrator constricted region having a third cross-sectional dimension that is fluidly coupled with the at least one sorter side channel; a concentrator expansion region having a fourth cross-sectional dimension that is larger than the third cross-sectional dimension, the concentrator expansion region being downstream from the concentrator constricted region; at least one concentrator side channel formed into a side of the concentrator expansion region; and at least one concentrator outlet that is downstream or medial from the at least one side channel. A platelet poor outlet is fluidly coupled with the at least one sorter outlet and/or the at least one concentrator side channel. A platelet concentrator outlet is fluidly coupled with the at least one concentrator outlet (e.g., downstream, medial or central from the concentrator side channel).

In some embodiments, a portable platelet apheresis system can include: a microfluidic network fluidly coupled with an outlet of the mixer, wherein the microfluidic network includes a whole blood sorter network and a platelet concentrator network downstream from the whole blood sorter. The whole blood sorter microfluidic network includes: a sorter constricted region having a first cross-sectional dimension; a sorter expansion region having a second cross-sectional dimension that is larger than the first cross-sectional dimension; at least one sorter side channel formed into a side of the sorter expansion region; and at least one sorter outlet that is downstream or medial from the at least one side channel. The platelet concentrator microfluidic network that includes: a concentrator constricted region having a third cross-sectional dimension; a concentrator expansion region having a fourth cross-sectional dimension that is larger than the third cross-sectional dimension; at least one concentrator side channel formed into a side of the concentrator expansion region; and at least one concentrator outlet that is downstream or medial from the at least one side channel. A platelet poor outlet is fluidly coupled with the at least one sorter outlet and/or the at least one concentrator side channel. A platelet concentrator outlet is fluidly coupled with the at least one concentrator outlet that is downstream or medial or central from the side channel. In some aspects, the sorter microfluidic network and platelet concentrator microfluidic network are in the same cartridge. In some aspects, the sorter microfluidic network and platelet concentrator microfluidic network are in different cartridges. In some aspects, the sorter microfluidic network is in a sorter body of the cartridge and the platelet concentrator microfluidic network are in a concentrator body of the same cartridge.

In some embodiments, the sorter microfluidic network is in a first body of the cartridge and the platelet concentrator microfluidic network are in a second body of the same cartridge. The cartridge includes an intermediate body with microfluidic channels that fluidly couple the sorter microfluidic network with the concentrator microfluidic network.

In some embodiments, a portable platelet apheresis system can include a pump fluidly coupled to the mixer and microfluidic network. A micro-controller can be operably coupled to a pump that is fluidly coupled to the mixer and microfluidic network.

In some embodiments, a casing (e.g., portable, like a hard brief case, pelican case, etc.) is adapted for containing the components of the portable platelet apheresis system. The casing can be a housing having a port adapted for removably receiving the cartridge. The casing can also contain a flowmeter fluidly coupled with the pump and the cartridge. In some aspects, the micro-controller is configured to receive flow date from the flowmeter and provide flow rate instruction data to the pump to obtain a desired flow rate. In some aspects, the micro-controller is configured to receive flow date from the flowmeter and change flow rate instruction data based on the flow data, and provide changed flow rate instruction data to the pump to obtain a changed flow rate.

In some embodiments, the portable platelet apheresis system can include: a series of whole blood sorter microfluidic networks in series; and at least one concentrator microfluidic network downstream from at least one of the whole blood sorter microfluidic networks. In some aspects, the system can include: a series of whole blood sorter microfluidic networks in series; and at least one concentrator microfluidic network downstream from each of the whole blood sorter microfluidic networks. In some aspects, a saline source fluidly coupled with an inlet of the sorter constricted region of a first whole blood sorter microfluidic network. In some aspects, the system includes a series of whole blood sorter microfluidic networks in series and one concentrator microfluidic network downstream from a last whole blood sorter microfluidic network of the series. In some aspects, the cartridge is configured as a disc cartridge.

In some embodiments, a whole blood sorter can include a whole blood sorter microfluidic network that includes: an inlet; a sorter constricted region having a first cross-sectional dimension downstream of the inlet; a sorter expansion region having a second cross-sectional dimension that is larger than the first cross-sectional dimension and that is downstream of the sorter constricted region; at least one sorter side channel formed into a side of the sorter expansion region; and at least one sorter outlet that is downstream or medial from the at least one side channel. In some aspects the inlet is configured to include whole blood during use. The expansion region includes platelets preferentially in side lateral regions thereof and includes white blood cells and red blood cells preferentially in a medial region between the two side lateral regions during use. The at least one sorter side channel includes a majority of separated platelets and a minority of separated white blood cells and separated red blood cells during use. The at least one sorter outlet includes a majority of separated white blood cells and separated red blood cells and a minority of separated platelets during use.

In some embodiments, the whole blood sorter can include two sorter side channels formed into opposite sides of the sorter expansion region and at least one sorter outlet are arranged laterally between the two sorter side channels. In some aspects, an inlet narrowing taper region is upstream of the sorter constricted region. In some aspects, the sorter expansion region includes an expanding section with a narrow inlet and expanded outlet. In some aspects, the sorter expansion region includes an expanding section with a narrow inlet and expanded outlet upstream of an expanded conduit section. In some aspects, the sorter expansion region includes an expanding section with a narrow inlet and expanded outlet upstream of an expanded conduit section has a longitudinal cross-section or longitudinal/latitudinal plane with a shape of a triangle.

In some embodiments, the whole blood sorter can include two sorter side channels formed into opposite sides of the sorter expansion region and a plurality of sorter outlets all arranged medially or centrally between the two sorter side channels. In some aspects, two sorter side channels are formed into opposite sides of the sorter expansion region and are outer channels, and a plurality of sorter outlets are all arranged between the two sorter side channels and are thereby considered inner channels between the two outer sorter side channels. In some aspects, the two sorter side channels are at opposite corners of the triangle shaped expansion region with the plurality of sorter outlets all arranged at the base of the triangle shaped expansion region between the opposite corners.

In some embodiments, a platelet concentrator can include a platelet concentrator microfluidic network that includes: an inlet; a concentrator constricted region having a first cross-sectional dimension downstream of the inlet; a concentrator expansion region having a second cross-sectional dimension that is larger than the first cross-sectional dimension and that is downstream of the concentrator constricted region; at least one concentrated side channel formed into a side of the concentrator expansion region; and at least one concentrator outlet that is downstream or medial or central from the at least one side channel. The inlet is configured to have platelet rich plasma when used. The expansion region is configured to include plasma preferentially in side lateral regions thereof and include platelets preferentially in a medial region between the two side lateral regions during use. The at least one concentrator side channel includes a majority of plasma and a minority of separated platelets during use. The at least one concentrator outlet includes a majority of separated platelets during use.

In some embodiments, two sorter concentrator channels are formed into opposite sides of the concentrator expansion region and at least one concentrator outlet is arranged between the two concentrator side channels. In some aspects, an inlet narrowing taper region is upstream of the concentrator constricted region. In some aspects, the concentrator expansion region includes an expanding section with a narrow inlet and expanded outlet. In some aspects, the concentrator expansion region includes an expanding section with a narrow inlet and expanded outlet upstream of an expanded conduit section. In some aspects, the concentrator expansion region includes an expanding section with a narrow inlet and expanded outlet upstream of an expanded conduit section has a longitudinal cross-section or longitudinal/latitudinal plane with a shape of a triangle or rectangle.

In some embodiments, two concentrator side channels are formed into opposite sides of the concentrator expansion region and a plurality of concentrator outlets are all arranged centrally or medially between the two concentrator side channels. The plurality of concentrator outlets can be arranged laterally with respect to each other.

In some embodiments, two concentrator side channels are formed into opposite sides of the concentrator expansion region and are outer channels, and a plurality of concentrator outlets are all arranged between the two concentrator side channels so as to be medial or central with respect to the two concentrator side channels. The concentrator outlets can be the inner channels that are medial or central or otherwise between the two outer channels. In some aspects, the two concentrator side channels are at opposite corners of the triangle or rectangle shaped expansion region with the plurality of concentrator outlets all arranged at the base of the triangle or rectangle shaped expansion region between the opposite corners. In some aspects, the concentrator outlets are the penultimate outlets when the side channels are the ultimate outlets.

In some embodiments, a method of separating platelets from whole blood can include: providing the portable platelet system of one of the embodiments; introducing whole blood into the whole blood inlet; mixing the whole blood with the anticoagulant; introducing the whole blood from the mixer into the whole blood sorter; collecting separated platelets from the at least one sorter side channel and/or the at least one concentrator outlet; and collecting separated white blood cells and red blood cells from the at least one sorter outlet. The methods can include collecting platelet poor plasma from the at least one concentrator side channel. The methods can also include controlling a flow rate through the microfluidic networks.

In some embodiments, a method of separating platelets from whole blood can include: proving the whole blood sorter of one of the embodiments; introducing whole blood into the sorter constricted region; flowing the whole blood through the sorter expansion region so as to preferentially distribute platelets at lateral sides and preferentially distribute white blood cells and red blood cells medially between the lateral sides; collecting separated platelets from the at least one sorter side channel; and collecting separated white blood cells and red blood cells from the at least one sorter outlet.

A method of separating platelets from platelet rich plasma can include: proving the platelet concentrator of one of the embodiments; introducing platelet rich plasma into the concentrator constricted region; flowing the platelet rich plasma through the constrictor expansion region so as to preferentially distribute platelets away from the lateral sides and preferentially distribute platelets medially between the lateral sides (flowing the platelet rich plasma through the constrictor expansion region so as to preferentially distribute platelets in a region that is laterally away from a centerline and medially away from the lateral sides); collecting separated plasma from the at least one concentrator side channel; and collecting separated platelets from the at least one concentrator outlet.

In certain embodiments, the present invention is directed to a composition comprising biological particles selected from the group consisting of: (a) viruses, including human viruses such as poliovirus (approximately 30 nm), influenza virus (approximately 80-120 nm), SARS-COV-2 (approximately 60-140 nm), herpesvirus (approximately 150-200 nm), and mimivirus (approximately 400-500 nm); (b) bacteriophages (phages), including Microviridae (approximately 24-30 nm), Leviviridae (approximately 27 nm), Inoviridae (approximately 6.5 nm wide and 900 nm long), Myoviridae (T4 phage, approximately 90 nm capsid with 100-200 nm tail), Siphoviridae (lambda phage, approximately 50-60 nm with 135 nm tail), and jumbo phages (KZ phage, approximately 200 nm capsid with 300 nm tail); (c) bacterial cells, including Gram-negative bacteria such as Escherichia coli (approximately 0.5-2.0 μm), Salmonella (approximately 0.7-1.5 μm), and Gram-positive bacteria such as Staphylococcus aureus (approximately 0.5-1.0 μm); (d) eukaryotic cells, including red blood cells (approximately 7-8 μm), white blood cells (approximately 10-15 μm), and platelets (approximately 2-3 μm); (e) nucleic acids, including single-stranded DNA (ssDNA, approximately 2 nm in diameter and variable length), double-stranded DNA (dsDNA, approximately 2 nm in diameter with lengths from 3,000 to over 500,000 base pairs), RNA (approximately 1-2 nm in diameter, varying from 20-200,000 nucleotides); (f) proteins, including small peptides (approximately 0.5-5 nm), globular proteins such as hemoglobin (approximately 5 nm), enzyme complexes such as ribosomes (approximately 20-30 nm), and structural proteins such as collagen fibrils (approximately 300 nm long, 1.5 nm in diameter); (g) antibodies, including immunoglobulin G (IgG, approximately 10 nm), immunoglobulin M (IgM, approximately 20-30 nm), and engineered antibody fragments (approximately 5-10 nm); (h) other biological particles, including exosomes (approximately 30-150 nm), microvesicles (approximately 100-1,000 nm), apoptotic bodies (approximately 500-5,000 nm), prions (approximately 5-10 nm), and viroids (approximately 2-10 nm).

The methods can be performed as described herein, such as in the examples.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In one embodiment, the present methods can include aspects performed on a computing system. As such, the computing system can include a memory device that has the computer-executable instructions for performing the method. The computer-executable instructions can be part of a computer program product that includes one or more algorithms for performing any of the methods of any of the claims.

In one embodiment, any of the operations, processes, methods, or steps described herein can be implemented as computer-readable instructions stored on a computer-readable medium. The computer-readable instructions can be executed by a processor of a wide range of computing systems from desktop computing systems, portable computing systems, tablet computing systems, hand-held computing systems as well as network elements, base stations, femtocells, and/or any other computing device.

There is little distinction left between hardware and software implementations of aspects of systems; the use of hardware or software is generally (but not always, in that in certain contexts the choice between hardware and software can become significant) a design choice representing cost vs. efficiency tradeoffs. There are various vehicles by which processes and/or systems and/or other technologies described herein can be affected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The foregoing detailed description has set forth various embodiments of the processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a CD, a DVD, a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those generally found in data computing/communication and/or network computing/communication systems.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

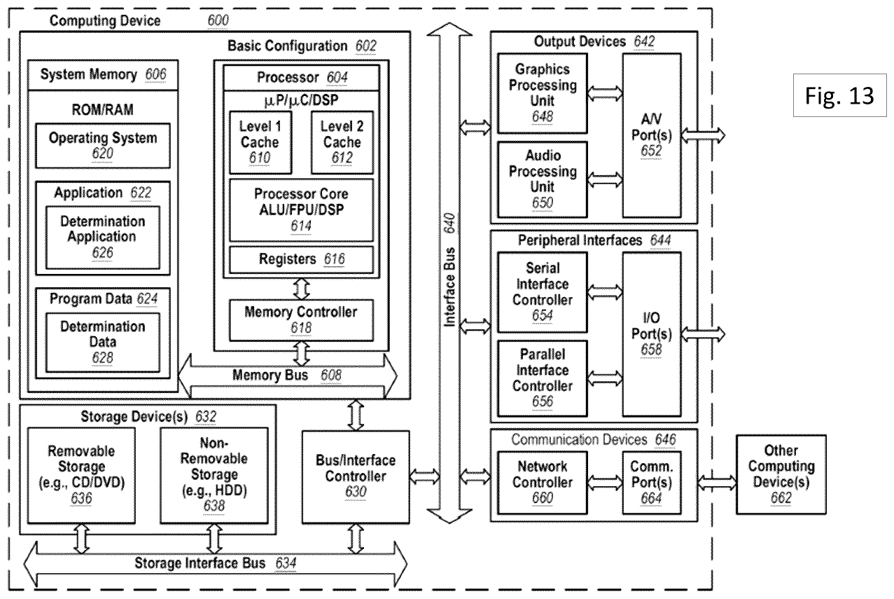

FIG. 13 shows an example computing device 600 that is arranged to perform any of the computing methods described herein. In a very basic configuration 602, computing device 600 generally includes one or more processors 604 and a system memory 606. A memory bus 608 may be used for communicating between processor 604 and system memory 606.

Depending on the desired configuration, processor 604 may be of any type including but not limited to a microprocessor (μP), a microcontroller (μC), a digital signal processor (DSP), or any combination thereof. Processor 604 may include one or more levels of caching, such as a level one cache 610 and a level two cache 612, a processor core 614, and registers 616. An example processor core 614 may include an arithmetic logic unit (ALU), a floating point unit (FPU), a digital signal processing core (DSP Core), or any combination thereof. An example memory controller 618 may also be used with processor 604, or in some implementations memory controller 618 may be an internal part of processor 604.

Depending on the desired configuration, system memory 606 may be of any type including but not limited to volatile memory (such as RAM), non-volatile memory (such as ROM, flash memory, etc.) or any combination thereof. System memory 606 may include an operating system 620, one or more applications 622, and program data 624. Application 622 may include a determination application 626 that is arranged to perform the functions as described herein including those described with respect to methods described herein. Program Data 624 may include determination information 628 that may be useful for analyzing the contamination characteristics provided by the sensor unit 240. In some embodiments, application 622 may be arranged to operate with program data 624 on operating system 620 such that the work performed by untrusted computing nodes can be verified as described herein. This described basic configuration 602 is illustrated in FIG. 6 by those components within the inner dashed line.

Computing device 600 may have additional features or functionality, and additional interfaces to facilitate communications between basic configuration 602 and any required devices and interfaces. For example, a bus/interface controller 630 may be used to facilitate communications between basic configuration 602 and one or more data storage devices 632 via a storage interface bus 634. Data storage devices 632 may be removable storage devices 636, non-removable storage devices 638, or a combination thereof. Examples of removable storage and non-removable storage devices include magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include volatile and nonvolatile, removable and non-removable media implemented in any method or technology for storage of information, such as computer readable instructions, data structures, program modules, or other data.

System memory 606, removable storage devices 636 and non-removable storage devices 638 are examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 600. Any such computer storage media may be part of computing device 600.

Computing device 600 may also include an interface bus 640 for facilitating communication from various interface devices (e.g., output devices 642, peripheral interfaces 644, and communication devices 646) to basic configuration 602 via bus/interface controller 630. Example output devices 642 include a graphics processing unit 648 and an audio processing unit 650, which may be configured to communicate to various external devices such as a display or speakers via one or more A/V ports 652. Example peripheral interfaces 644 include a serial interface controller 654 or a parallel interface controller 656, which may be configured to communicate with external devices such as input devices (e.g., keyboard, mouse, pen, voice input device, touch input device, etc.) or other peripheral devices (e.g., printer, scanner, etc.) via one or more I/O ports 658. An example communication device 646 includes a network controller 660, which may be arranged to facilitate communications with one or more other computing devices 662 over a network communication link via one or more communication ports 664.

The network communication link may be one example of a communication media. Communication media may generally be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) and other wireless media. The term computer readable media as used herein may include both storage media and communication media.

Computing device 600 may be implemented as a portion of a small-form factor portable (or mobile) electronic device such as a cell phone, a personal data assistant (PDA), a personal media player device, a wireless web-watch device, a personal headset device, an application specific device, or a hybrid device that include any of the above functions. Computing device 600 may also be implemented as a personal computer including both laptop computer and non-laptop computer configurations. The computing device 600 can also be any type of network computing device. The computing device 600 can also be an automated system as described herein.

The embodiments described herein may include the use of a special purpose or general-purpose computer including various computer hardware or software modules.

Embodiments within the scope of the present invention also include computer-readable media for carrying or having computer-executable instructions or data structures stored thereon. Such computer-readable media can be any available media that can be accessed by a general purpose or special purpose computer. By way of example, and not limitation, such computer-readable media can comprise RAM, ROM, EEPROM, CD-ROM or other optical disk storage, magnetic disk storage or other magnetic storage devices, or any other medium which can be used to carry or store desired program code means in the form of computer-executable instructions or data structures and which can be accessed by a general purpose or special purpose computer. When information is transferred or provided over a network or another communications connection (either hardwired, wireless, or a combination of hardwired or wireless) to a computer, the computer properly views the connection as a computer-readable medium. Thus, any such connection is properly termed a computer-readable medium. Combinations of the above should also be included within the scope of computer-readable media.

Computer-executable instructions comprise, for example, instructions and data which cause a general purpose computer, special purpose computer, or special purpose processing device to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

As used herein, the term “module” or “component” can refer to software objects or routines that execute on the computing system. The different components, modules, engines, and services described herein may be implemented as objects or processes that execute on the computing system (e.g., as separate threads). While the system and methods described herein are preferably implemented in software, implementations in hardware or a combination of software and hardware are also possible and contemplated. In this description, a “computing entity” may be any computing system as previously defined herein, or any module or combination of modulates running on a computing system.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety.

1. A microfluidic separation system comprising:
an inlet configured to receive a carrier fluid from a carrier fluid source, the carrier fluid including first particles;
a carrier fluid sorter coupled to the inlet, the carrier fluid sorter including a carrier fluid sorter microfluidic network having:
a sorter constricted region having a first cross-sectional dimension;
a sorter expansion region having a second cross-sectional dimension that is larger than the first cross-sectional dimension;
at least one sorter side channel formed into a side of the sorter expansion region, the at least one sorter side channel configured to receive a first fraction of the carrier fluid, the first fraction being first particle poor; and
at least one sorter outlet that is downstream or medial from the at least one sorter side channel, the at least one sorter outlet configured to receive a second fraction of the carrier fluid, the second fraction being first particle concentrated; and
wherein the carrier fluid sorter microfluidic network is configured to direct the first fraction of the carrier fluid to the at least one sorter side channel and configured to direct the second fraction of the carrier fluid to the at least one sorter outlet.

2. The microfluidic separation system of claim 1, wherein the carrier fluid further includes second particles that are smaller than the first particles, the first fraction being second particle concentrated and the second fraction being second particle poor.

3. The microfluidic separation system of claim 1, wherein the sorter constricted region is defined by a width between about 2 micrometers and about 30 micrometers, a length between about 0.2 micrometers and about 2 centimeters, and a height between about 10 micrometers and about 500 micrometers.

4. The microfluidic separation system of claim 1, wherein a flow rate through the carrier fluid sorter microfluidic network is between about 2 microliters per minute and about 1 milliliter per minute.

5. The microfluidic separation system of claim 1, wherein the first particles are virus particles.

6. The microfluidic separation system of claim 5, wherein the carrier fluid further includes second particles that are smaller than the first particles, the first fraction being second particle concentrated and the second fraction being second particle poor.

7. The microfluidic separation system of claim 6, wherein the second particles are endotoxins or different virus particles.

8. The microfluidic separation system of claim 5, wherein at least one of:
the sorter constricted region has a width between about 2 micrometers and about 10 micrometers;
the sorter constricted region has a length between about 0.2 millimeters and about 2 centimeters; or
the sorter constricted region has a height between about 10 micrometers and about 250 micrometers.

9. The microfluidic separation system of claim 2, wherein the first particles are a first group of virus particles and the second particles are a second group of virus particles, the first group of virus particles being larger than the second group of virus particles.

10. The microfluidic separation system of claim 9, wherein at least one of:
the sorter constricted region has a width between about 2 micrometers and about 10 micrometers;
the sorter constricted region has a length between about 0.2 millimeters and about 2 centimeters; or
the sorter constricted region has a height between about 10 micrometers and about 250 micrometers.

11. The microfluidic separation system of claim 9, wherein the first group of virus particles have a size between about 250 nanometers and about 530 nanometers and the second group of virus particles have a size between about 40 nanometers and about 300 nanometers.

12. The microfluidic separation system of claim 1, wherein the first particles include bacteria.

13. The microfluidic separation system of claim 12, wherein the carrier fluid further includes second particles that are smaller than the first particles, the first fraction being second particle concentrated and the second fraction being second particle poor.

14. The microfluidic separation system of claim 13, wherein the second particles are bacteriophages.

15. The microfluidic separation system of claim 13, wherein a height of the at least one sorter side channel is lesser than a height of the at least one sorter outlet to increase fluidic resistance provided by the at least one sorter side channel such that the first fraction of the carrier fluid is directed to the at least one sorter side channel and the second fraction of the carrier fluid is directed to the at least one sorter outlet.

16. The microfluidic separation system of claim 13, wherein at least one of:
the sorter constricted region has a width between about 7 micrometers and about 20 micrometers;
the sorter constricted region has a length between about 0.2 millimeters and about 4 centimeters; or
the sorter constricted region has a height between about 40 micrometers and about 500 micrometers.

17. The microfluidic separation system of claim 1, wherein the first particles are liposomes, extracellular vesicles (EV), or exosomes.

18. The microfluidic separation system of claim 17, wherein the carrier fluid further includes second particles that are smaller than the first particles, the first fraction being second particle concentrated and the second fraction being second particle poor.

19. The microfluidic separation system of claim 18, wherein the second particles are nucleic acids, proteins, or polypeptides.

20. The microfluidic separation system of claim 18, wherein at least one of:
the sorter constricted region has a width between about 2 micrometers and about 10 micrometers;
the sorter constricted region has a length between about 0.2 millimeters and about 2 centimeters; or
the sorter constricted region has a height between about 10 micrometers and about 250 micrometers.

21. The microfluidic separation system of claim 1, wherein the carrier fluid is whole blood, the first particles are red blood cells and white blood cells and wherein the carrier fluid includes second particles that are different than the first particles, the first fraction being second particle concentrated and the second fraction being second particle poor.

22. The microfluidic separation system of claim 21, wherein the second particles are platelets.

23. The microfluidic separation system of claim 21, wherein at least one of:
the sorter constricted region has a width between about 10 micrometers and about 30 micrometers;
the sorter constricted region has a length between about 0.5 millimeters and about 30 millimeters; or
the sorter constricted region has a height between about 25 micrometers and about 500 micrometers.

24. The microfluidic separation system of claim 1, wherein the carrier fluid is platelet-rich plasma and the first particles are platelets, and wherein the sorter constricted region has at least one of: a width between about 10 micrometers and about 30 micrometers, a length between about 0.5 millimeters and about 30 millimeters, or a height between about 25 micrometers and about 500 micrometers.

25. A method of separating particles, the method comprising:
receiving carrier fluid at an inlet of a carrier fluid sorter, the carrier fluid including first particles;
fractionating the carrier fluid into a first fraction and a second fraction, the first fraction being first particle poor and the second fraction being first particle concentrated;
directing the first fraction to at least one sorter side channel of the carrier fluid sorter; and
directing the second fraction to at least one sorter outlet that is downstream or medial from the at least one sorter side channel.

26. The method of separating particles of claim 25, wherein the carrier fluid includes second particles that are smaller than the first particles, the first fraction is second particle concentrated and the second fraction is second particle poor.

27. The method of separating particles of claim 26, wherein:
the first particles are virus particles and the second particles are endotoxins or different virus particles;
the first particles are a first group of virus particles and the second particles are a second group of virus particles, the first group of particles being larger than the second group of particles;
the first particles are bacteria and the second particles are bacteriophages;
the first particles are liposomes, extracellular vesicles (EV), or exosomes and the second particles are nucleic acids, proteins, or polypeptides; or
the first particles are red blood cells and white bloods cells and the second particles are platelets.

28. The method of claim 25, wherein the fractionation of the carrier fluid is attributable, at least in part, to a sorter constricted region downstream of the inlet and upstream of the at least one sorter side channels and the at least one sorter outlet, the sorter constricted region being defined by a width between about 2 micrometers and about 30 micrometers, a length between about 0.2 micrometers and about 2 centimeters, and a height between about 10 micrometers and about 500 micrometers.
